(19) United States
(12) Patent Application Publication
(10) Pub. No.: US 2008/0207632 A1
(43) Pub. Date: Aug. 28, 2008

(54) PROTEIN KINASE INHIBITORS

(75) Inventors: David J. Bearss, Cedar Hills, UT (US); Cory L. Grand, Salt Lake City, UT (US); Xiao-Hui Liu, Salt Lake City, UT (US); Hariprasad Vankayalapati, Draper, UT (US)

Correspondence Address: SEED INTELLECTUAL PROPERTY LAW GROUP PLLC, 701 FIFTH AVE, SUITE 5400, SEATTLE, WA, 98104, US

(73) Assignee: SUPERGEN, INC., Dublin, CA (US)

(21) Appl. No.: 11/932,617

(22) Filed: Oct. 31, 2007

Related U.S. Application Data
(60) Provisional application No. 60/911,828, filed on Apr. 13, 2007.
(60) Provisional application No. 60/892,520, filed on Mar. 1, 2007.
(60) Provisional application No. 60/855,819, filed on Oct. 31, 2006.

(51) Int. Cl. A61K 31/496; C07D 487/04; A61P 35/00

(52) U.S. Cl. 514/252.17; 544/250

(57) ABSTRACT

Protein kinase inhibitors are disclosed having utility in the treatment of protein kinase-mediated diseases and conditions, such as cancer, such as Aurora kinase-expressing cancers and Axl kinase-expressing cancers. Compounds of the invention have the following structure:

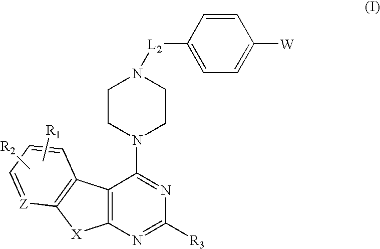

including stereoisomers, prodrugs and pharmaceutically acceptable salts thereof, wherein R₁, R₂, R₃, X, Z, L₂ and w are as defined herein. Also disclosed are compositions containing a compound of this invention, as well as methods relating to the use thereof.

### CROSS-REFERENCE TO RELATED APPLICATIONS

This application claims the benefit under 35 U.S.C. § 119(e) of U.S. Provisional Patent Application No. 60/911,828, filed Apr. 13, 2007; U.S. Provisional Patent Application No. 60/892,520, filed Mar. 1, 2007; and U.S. Provisional Patent Application No. 60/855,819, filed Oct. 31, 2006, where these three provisional applications are incorporated herein by reference in their entireties.

### BACKGROUND OF THE INVENTION

1. Field of the Invention

The present invention relates, in general, to compounds that inhibit protein kinase activity, and to compositions and methods related thereto.

2. Description of the Related Art

Cancer (and other hyperproliferative diseases) is characterized by uncontrolled cell proliferation. This loss of the normal control of cell proliferation often appears to occur as the result of genetic damage to cell pathways that control progress through the cell cycle. The cell cycle consists of DNA synthesis (S phase), cell division or mitosis (M phase), and non-synthetic periods referred to as gap 1 (G1) and gap 2 (G2). The M-phase is composed of mitosis and cytokinesis (separation into two cells). All steps in the cell cycle are controlled by an orderly cascade of protein phosphorylation and several families of protein kinases are involved in carrying out these phosphorylation steps. In addition, the activity of many protein kinases increases in human tumors compared to normal tissue and this increased activity can be due to many factors, including increased levels of a kinase or changes in expression of co-activators or inhibitory proteins.

Cells have proteins that govern the transition from one phase of the cell cycle to another. For example, the cyclins are a family of proteins whose concentrations increase and decrease throughout the cell cycle. The cyclins turn on, at the appropriate time, different cyclin-dependent protein kinases (CDKs) that phosphorylate substrates essential for progression through the cell cycle. Activity of specific CDKs at specific times is essential for both initiation and coordinated progress through the cell cycle. For example, CDK1 is the most prominent cell cycle regulator that orchestrates M-phase activities. However, a number of other mitotic protein kinases that participate in M-phase have been identified, which include members of the polo, aurora, and NIMA (Never-In-Mitosis-A) families and kinases implicated in mitotic checkpoints, mitotic exit, and cytokinesis.

Aurora kinases are a family of oncogenic serine/threonine kinases that localize to the mitotic apparatus (centrosome, poles of the bipolar spindle, or midbody) and regulate completion of centrosome separation, bipolar spindle assembly and chromosome segregation. Three human homologs of aurora kinases have been identified (aurora-1 (also known as aurora-B), aurora-2 (also known as aurora-A) and aurora-3 (also known as aurora-C). They all share a highly conserved catalytic domain located in the carboxyl terminus, but their amino terminal extensions are of variable lengths with no sequence similarity.

The individual roles of the three Aurora kinases is becoming better understood. Aurora-2 kinase plays its major part at early mitosis, where it is involved in the creation of a proper (bipolar) spindle apparatus, and also directs the separation of centrosomes to these two poles [1-5]. Aurora-1 kinase plays a role later in mitosis, and takes part in orienting the chromosomes appropriately to the spindle, allowing the cell to pass through the spindle assembly checkpoint. It is also involved in separation of the cell contents after chromosome segregation has taken place [5-8]. The purpose of Aurora-3 in the cell cycle has yet to be fully understood, but has been shown to have a complementary role to that of Aurora-1, and can interfere with its function if overexpressed [9, 10].

Aurora-2 and Aurora-1 are known to be overexpressed in a variety of tumor types, including pancreas, colorectal, ovarian, prostate and breast cancers, as well as glioblastomas and astrocyomas [3, 11-17], suggesting that either or both play an important role in cancer. However, it has been suggested that Aurora-2 is a more attractive cancer drug target, given that its abrogation leads to brief mitotic arrest followed immediately by apoptosis [5].

Consistent with the importance of Aurora-2 kinase in cancer, it acts as an oncogene and transforms both Rat1 fibroblasts and mouse NIH3T3 cells in vitro, and transforms NIH 3T3 cells grown as tumors in nude mice. Excess aurora-2 may drive cells to aneuploidy (abnormal numbers of chromosomes) by accelerating the loss of tumor suppressor genes and/or amplifying oncogenes, events known to contribute to cellular transformation. Cells with excess aurora-2 may escape mitotic check points, which in turn can activate proto-oncogenes inappropriately. In additional, aurora-2 kinase antisense oligonucleotide treatment has been shown to cause cell cycle arrest and increased apoptosis. Therefore, aurora-2 kinase is an attractive target for rational design of novel small molecule inhibitors for the treatment of cancer and other conditions.

Axl is a receptor tyrosine kinase (ligand: Growth Arrest Specific protein 6, Gas6) which is unique in having two tandem immunoglobulin-like repeats and two fibronectin type III repeats, a feature common in cellular adhesion molecules. For this reason, it has a family of its own, the Axl/Ufo subfamily of tyrosine kinases. The expression of Axl/Gas6 has been shown in a number of human malignancies, including ovarian, melanoma, renal cell carcinoma, uterine leiomyoma, uterine endometrial cancer, thyroid carcinoma, gastric cancer, breast cancer, NSCLC, CML, AML, colorectal carcinoma, prostate cancer, various lymphomas, and esophageal cancer. The Axl proto-oncogene is therefore also an attractive and valuable target for the discovery and development of new therapeutic agents.

Quinazoline derivatives have been proposed for inhibiting protein kinase activity. For example, WO 96/09294, WO 96/33981 and EP 0837 063 describe the use of certain quinazoline compounds as receptor tyrosine kinase inhibitors. In addition, WO 01/21596 proposes the use of quinazoline derivatives to inhibit aurora-2 kinase.

What remains needed, however, are additional and improved inhibitors of protein kinase activity, such as inhibitors of aurora-kinase activity and/or Axl kinase activity. In addition, there is a need for inhibitors that are specific and selective for individual members of the aurora kinase family, particularly aurora-2 kinase. The present invention fulfills these needs and offers other related advantages.

### BRIEF SUMMARY OF THE INVENTION

The present invention is generally directed to compounds having the following general structure (I):

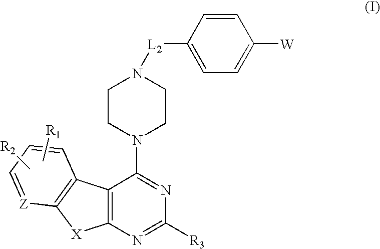

including stereoisomers, prodrugs and pharmaceutically acceptable salts thereof, wherein R₁, R₂, R₃, X, Z, L₂ and w are as defined herein.

These compounds of the present invention have utility over a broad range of therapeutic applications, and may be used to treat diseases, such as cancer, that are mediated at least in part by protein kinase activity. Accordingly, in one aspect of the invention, the compounds described herein are formulated as pharmaceutically acceptable compositions for administration to a subject in need thereof.

In another aspect, the invention provides methods for treating or preventing a protein kinase-mediated disease, such as cancer, which method comprises administering to a patient in need of such a treatment a therapeutically effective amount of a compound described herein or a pharmaceutically acceptable composition comprising said compound. In certain embodiments, the protein kinase-mediated disease is an aurora-2 kinase-mediated disease, such as an aurora-2 kinase-expressing cancer. In other embodiments, the protein kinase-mediated disease is an Axl kinase-mediated disease, such as an Axl kinase-expressing cancer. In certain more particular embodiments, the Aurora kinase- or Axl kinase-expressing cancer is selected from uterine/cervix squamous cell carcinoma, kidney cancer, small cell lung carcinoma, hepatocellular carcinoma, non-small cell lung carcinoma, melanoma, infiltrating ductal breast cancer, infiltrating lobular breast cancer, lung adenocarcinoma, colorectal cancer, stomach adenocarcinoma, or ovarian adenocarcinoma.

Another aspect of the invention relates to inhibiting protein kinase activity in a biological sample, which method comprises contacting the biological sample with a compound described herein, or a pharmaceutically acceptable composition comprising said compound. In certain embodiments, the protein kinase is aurora-2 kinase. In other embodiments, the protein kinase is an Axl kinase.

Another aspect of this invention relates to a method of inhibiting protein kinase activity in a patient, which method comprises administering to the patient a compound described herein or a pharmaceutically acceptable composition comprising said compound. In certain embodiments, the protein kinase is aurora-2 kinase. In other embodiments, the protein kinase is an Axl kinase.

Administering a compound of the invention to a patient may be achieved by any suitable means. In certain embodiments, the administration is oral, subcutaneous or intravenous. In certain other embodiments, the administering step comprises administering the compound prior to treatment with one or more other chemotherapeutic agents, or in combination with one or more other chemotherapeutic agents. In a particular embodiment, the other chemotherapeutic agent is paclitaxel.

Another aspect of the invention relates to methods for specifically and selectively inhibiting the activity of Aurora-2 kinase, without inhibiting the activity of other Aurora kinase family members, particularly, Aurora-1 kinase. Such methods have the potential to result in reduced toxicity and/or improved efficacy.

These and other aspects of the invention will be apparent upon reference to the following detailed description and attached figures. To that end, certain patent and other documents are cited herein to more specifically set forth various aspects of this invention. Each of these documents is hereby incorporated by reference in its entirety.

### BRIEF DESCRIPTION OF THE DRAWINGS

FIG. 1 shows the in vivo anti-tumor activity of an illustrative compound of the present invention.

FIG. 2 shows the activity of an illustrative compound of the invention against aurora-A (aurora-2) kinase and aurora-B (aurora 1).

FIG. 3 shows the cell cycle distribution characteristics of A549 cells following treatment with an illustrative compound of the invention.

FIG. 4 shows the increased histone H3 phosphorylation observed following treatment with an illustrative compound of the invention.

FIG. 5 shows the effect of compound 2-3 on tumor growth inhibition of HT-29 colon cancer xenografts.

FIG. 6 shows the effect of compound 2-3 on tumor growth inhibition of DU-145 prostate cancer xenografts.

### DETAILED DESCRIPTION OF THE INVENTION

According to a general aspect of the present invention, there are provided compounds useful as protein kinase inhibitors and compositions and methods relating thereto. Compounds of the invention have the following structure (I) below:

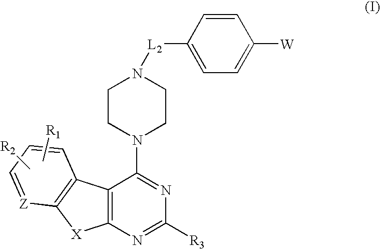

including stereoisomers and pharmaceutically acceptable salts thereof, wherein:

X is NH, S or O;

Z is CH or N;

R₁ and R₂ are the same or different and are independently hydrogen, hydroxyl, halo, —CN, —NO₂, —NH₂, —R, —OR, —SCH₃, —CF₃, morpholine, —C(═O)OR, —OC(═O)R, where R is alkyl or substituted alkyl; or —O(CH₂)ₙ—Rₓ, where n is 2-4 and Rₓ is N-methylpiperazine, morpholine or 2-methylpyrrolidine.

R₃ is hydrogen, —NH₂, alkyl, —CN, or —NO₂, or R₃ is -L₃-Cycl₃ wherein L₃ is a direct bond, —S— or —NH—, and CyCl₃ is a carbocycle, substituted carbocycle, heterocycle or substituted heterocycle;

L₂ is —C(═S)NH—,

W is:

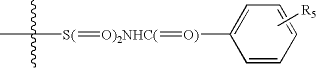

or —S(═O)₂NHR_{y}, where R_{y} is

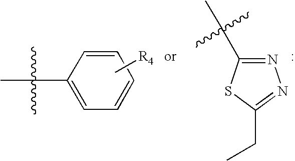

R₄ is alkyl, halo, or haloalkyl; and R₅ is hydrogen, alkyl, alkoxy, haloalkyl, halo, hydroxyl, or substituted alkoxy.

In another general aspect, the present invention relates to methods for inhibiting Axl kinase activity, for inhibiting Axl kinase-mediated cell proliferation and for treating Axl kinase-mediated diseases, such as cancer, using a compound having the following structure (I):

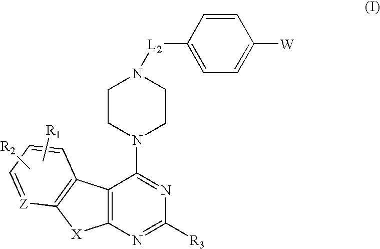

including stereoisomers, prodrugs and pharmaceutically acceptable salts thereof, wherein:

X is NH, S or O;

Z is CH or N;

R₁ and R₂ are the same or different and are independently hydrogen, hydroxyl, halo, —CN, —NO₂, —NH₂, —R, —OR, —SCH₃, —CF₃, morpholine, —C(═O)OR, —OC(═O)R, where R is alkyl or substituted alkyl; or —O(CH₂)ₙ—Rₓ, where n is 2-4 and Rₓ is N-methylpiperazine, morpholine or 2-methylpyrrolidine.

R₃ is hydrogen, —NH₂, alkyl, —CN, or —NO₂, or R₃ is -L₃-Cycl₃ wherein L₃ is a direct bond, —S— or —NH—, and CyCl₃ is a carbocycle, substituted carbocycle, heterocycle or substituted heterocycle;

L₂ is —C(═S)NH—, —NHC(═S)—, —NHC(═S)NH—, —C(═O)NH—, —NHC(═O)—, —NHC(═O)NH—, —(CH₂)ₙ—, —NH(CH₂)ₙ—, —(CH₂)ₙNH—, —NH(CH₂)ₙNH—, —C(═S)NH(CH₂)ₙ—, —NHC(═S)(CH₂)ₙ—, —(CH₂)ₙC(═S)NH(CH₂)ₙ—, —(CH₂)ₙNHC(═S)(CH₂)ₙ—, —NHC(═O)—, —S(═O)₂—, —S(═O)₂NH—, —NHS(═O)₂—, wherein n is, at each occurrence the same or different and independently 1, 2, 3 or 4; and

w is —S(═O)₂NHC(═O)CH₃, —NHC(═O)R_{y}, —C(═O)NH—, —NHS(═O)₂R_{y}, —S(═O)₂NHR_{y}— where R_{y} is alkyl, substituted alkyl, cycloalkyl, substituted alkyl, carbocycle, substituted carbocycle, heterocycle, substituted heterocycle, —NH₂, —NH₂.HCl, and —S(═O)₂—R_{z}, where R_{z} is selected from alkyl, substituted alkyl, amine, N-methylpiperazine, morpholine, 2-methylpyrrolidine.

Unless otherwise stated the following terms used in the specification and claims have the meanings discussed below:

“Alkyl” refers to a saturated straight or branched hydrocarbon radical of one to six carbon atoms, preferably one to four carbon atoms, e.g., methyl, ethyl, propyl, 2-propyl, n-butyl, iso-butyl, tert-butyl, pentyl, hexyl, and the like, preferably methyl, ethyl, propyl, or 2-propyl. Representative saturated straight chain alkyls include methyl, ethyl, n-propyl, n-butyl, n-pentyl, n-hexyl, and the like; while saturated branched alkyls include isopropyl, sec-butyl, isobutyl, tert-butyl, isopentyl, and the like. Representative saturated cyclic alkyls include cyclopropyl, cyclobutyl, cyclopentyl, cyclohexyl, —CH₂-cyclohexyl, and the like; while unsaturated cyclic alkyls include cyclopentenyl, cyclohexenyl, —CH₂-cyclohexenyl, and the like. Cyclic alkyls are also referred to herein as a “cycloalkyl.” Unsaturated alkyls contain at least one double or triple bond between adjacent carbon atoms (referred to as an “alkenyl” or “alkynyl”, respectively.) Representative straight chain and branched alkenyls include ethylenyl, propylenyl, 1-butenyl, 2-butenyl, isobutylenyl, 1-pentenyl, 2-pentenyl, 3-methyl-1-butenyl, 2-methyl-2-butenyl, 2,3-dimethyl-2-butenyl, and the like; while representative straight chain and branched alkynyls include acetylenyl, propynyl, 1-butynyl, 2-butynyl, 1-pentynyl, 2-pentynyl, 3-methyl-1-butynyl, and the like.

“Alkylene” means a linear saturated divalent hydrocarbon radical of one to six carbon atoms or a branched saturated divalent hydrocarbon radical of three to six carbon atoms, e.g., methylene, ethylene, 2,2-dimethylethylene, propylene, 2-methylpropylene, butylene, pentylene, and the like, preferably methylene, ethylene, or propylene.

“Cycloalkyl” refers to a saturated cyclic hydrocarbon radical of three to eight carbon atoms, e.g., cyclopropyl, cyclobutyl, cyclopentyl or cyclohexyl.

“Alkoxy” means a radical —ORₐ where Rₐ is an alkyl as defined above, e.g., methoxy, ethoxy, propoxy, butoxy and the like.

“Halo” means fluoro, chloro, bromo, or iodo, preferably fluoro and chloro.

“Haloalkyl” means alkyl substituted with one or more, preferably one, two or three, same or different halo atoms, e.g., —CH₂Cl, —CF₃, —CH₂CF₃, —CH₂CCl₃, and the like.

“Haloalkoxy” means a radical —OR_{b} where R_{b} is an haloalkyl as defined above, e.g., trifluoromethoxy, trichloroethoxy, 2,2-dichloropropoxy, and the like.

“Acyl” means a radical —C(O)R_{c} where R_{c} is hydrogen, alkyl, or haloalkyl as defined herein, e.g., formyl, acetyl, trifluoroacetyl, butanoyl, and the like.

“Aryl” refers to an all-carbon monocyclic or fused-ring polycyclic (i.e., rings which share adjacent pairs of carbon atoms) groups of 6 to 12 carbon atoms having a completely conjugated pi-electron system. Examples, without limitation, of aryl groups are phenyl, naphthyl and anthracenyl. The aryl group may be substituted or unsubstituted. When substituted, the aryl group is substituted with one or more, more preferably one, two or three, even more preferably one or two substituents independently selected from the group consisting of alkyl (wherein the alkyl may be optionally substituted with one or two substituents), haloalkyl, halo, hydroxy, alkoxy, mercapto, alkylthio, cyano, acyl, nitro, phenoxy, heteroaryl, heteroaryloxy, haloalkyl, haloalkoxy, carboxy, alkoxycarbonyl, amino, alkylamino dialkylamino, aryl, heteroaryl, carbocycle or heterocycle (wherein the aryl, heteroaryl, carbocycle or heterocycle may be optionally substituted).

“Heteroaryl” refers to a monocyclic or fused ring (i.e., rings which share an adjacent pair of atoms) group of 5 to 12 ring atoms containing one, two, three or four ring heteroatoms selected from N, O, or S, the remaining ring atoms being C, and, in addition, having a completely conjugated pi-electron system. Examples, without limitation, of unsubstituted heteroaryl groups are pyrrole, furan, thiophene, imidazole, oxazole, thiazole, pyrazole, pyridine, pyrimidine, quinoline, isoquinoline, purine, triazole, tetrazole, triazine, and carbazole. The heteroaryl group may be substituted or unsubstituted. When substituted, the heteroaryl group is substituted with one or more, more preferably one, two or three, even more preferably one or two substituents independently selected from the group consisting of alkyl (wherein the alkyl may be optionally substituted with one or two substituents), haloalkyl, halo, hydroxy, alkoxy, mercapto, alkylthio, cyano, acyl, nitro, haloalkyl, haloalkoxy, carboxy, alkoxycarbonyl, amino, alkylamino dialkylamino, aryl, heteroaryl, carbocycle or heterocycle (wherein the aryl, heteroaryl, carbocycle or heterocycle may be optionally substituted).

“Carbocycle” refers to a saturated, unsaturated or aromatic ring system having 3 to 14 ring carbon atoms. The term “carbocycle”, whether saturated or partially unsaturated, also refers to rings that are optionally substituted. The term “carbocycle” includes aryl. The term “carbocycle” also includes aliphatic rings that are fused to one or more aromatic or nonaromatic rings, such as in a decahydronaphthyl or tetrahydronaphthyl, where the radical or point of attachment is on the aliphatic ring. The carbocycle group may be substituted or unsubstituted. When substituted, the carbocycle group is substituted with one or more, more preferably one, two or three, even more preferably one or two substituents independently selected from the group consisting of alkyl (wherein the alkyl may be optionally substituted with one or two substituents), haloalkyl, halo, hydroxy, alkoxy, mercapto, alkylthio, cyano, acyl, nitro, haloalkyl, haloalkoxy, carboxy, alkoxycarbonyl, amino, alkylamino dialkylamino, aryl, heteroaryl, carbocycle or heterocycle (wherein the aryl, heteroaryl, carbocycle or heterocycle may be optionally substituted).

“Heterocycle” refers to a saturated, unsaturated or aromatic cyclic ring system having 3 to 14 ring atoms in which one, two or three ring atoms are heteroatoms selected from N, O, or S(O)ₘ (where m is an integer from 0 to 2), the remaining ring atoms being C, where one or two C atoms may optionally be replaced by a carbonyl group. The term “heterocycle” includes heteroaryl. The heterocyclyl ring may be optionally substituted independently with one or more, preferably one, two, or three substituents selected from alkyl (wherein the alkyl may be optionally substituted with one or two substituents), haloalkyl, cycloalkylamino, cycloalkylalkyl, cycloalkylaminoalkyl, cycloalkylalkylaminoalkyl, cyanoalkyl, halo, nitro, cyano, hydroxy, alkoxy, amino, alkylamino, dialkylamino, hydroxyalkyl, carboxyalkyl, aminoalkyl, alkylaminoalkyl, dialkylaminoalkyl, aralkyl, heteroaralkyl, aryl, heteroaryl, carbocycle, heterocycle (wherein the aryl, heteroaryl, carbocycle or heterocycle may be optionally substituted), aralkyl, heteroaralkyl, saturated or unsaturated heterocycloamino, saturated or unsaturated heterocycloaminoalkyl, and —COR_{d} (where R_{d} is alkyl). More specifically the term heterocyclyl includes, but is not limited to, tetrahydropyranyl, 2,2-dimethyl-1,3-dioxolane, piperidino, N-methylpiperidin-3-yl, piperazino, N-methylpyrrolidin-3-yl, pyrrolidino, morpholino, 4-cyclopropylmethylpiperazino, thiomorpholino, thiomorpholino-1-oxide, thiomorpholino-1,1-dioxide, 4-ethyloxycarbonylpiperazino, 3-oxopiperazino, 2-imidazolidone, 2-pyrrolidinone, 2-oxohomopiperazino, tetrahydropyrimidin-2-one, and the derivatives thereof. In certain embodiments, the heterocycle group is optionally substituted with one or two substituents independently selected from halo, alkyl, alkyl substituted with carboxy, ester, hydroxy, alkylamino, saturated or unsaturated heterocycloamino, saturated or unsaturated heterocycloaminoalkyl, or dialkylamino.

“Optional” or “optionally” means that the subsequently described event or circumstance may but need not occur, and that the description includes instances where the event or circumstance occurs and instances in which it does not. For example, “heterocyclic group optionally substituted with an alkyl group” means that the alkyl may but need not be present, and the description includes situations where the heterocycle group is substituted with an alkyl group and situations where the heterocycle group is not substituted with the alkyl group.

Lastly, the term “substituted” as used herein means any of the above groups (e.g., alkyl, aryl, heteroaryl, carbocycle, heterocycle, etc.) wherein at least one hydrogen atom is replaced with a substituent. In the case of an oxo substituent (“═O”) two hydrogen atoms are replaced. “Substituents” within the context of this invention include halogen, hydroxy, oxo, cyano, nitro, amino, alkylamino, dialkylamino, alkyl, alkoxy, thioalkyl, haloalkyl, hydroxyalkyl, aryl, substituted aryl, arylalkyl, substituted arylalkyl, heteroaryl, substituted heteroaryl, heteroarylalkyl, substituted heteroarylalkyl, heterocycle, substituted heterocycle, heterocyclealkyl, substituted heterocyclealkyl, —NRₑR_{f}, —NRₑC(═O)R_{f}, —NRₑC(═O)NRₑR_{f}, —NRₑC(═O)OR_{f}, —NRₑSO₂R_{f}, —ORₑ, —C(═O)Rₑ, —C(═O)ORₑ, —C(═O)NRₑR_{f}, —OC(═O)NRₑR_{f}, —SH, —SRₑ, —SORₑ, —S(═O)₂Rₑ, —OS(═O)₂Rₑ, —S(═O)₂ORₑ, wherein Rₑ and R_{f} are the same or different and independently hydrogen, alkyl, haloalkyl, substituted alkyl, aryl, substituted aryl, arylalkyl, substituted arylalkyl, heteroaryl, substituted heteroaryl, heteroarylalkyl, substituted heteroarylalkyl, heterocycle, substituted heterocycle, heterocyclealkyl or substituted heterocyclealkyl.

Certain illustrative compounds according to structure (I) for use as described herein are set forth below.

For example, in a more specific aspect of structure (I) above, X is NH and Z is CH.

In a more specific aspect of structure (I) above, R₁, R₂ and R₃ are selected from hydrogen, —NH₂, —OCH₃, —OH, —CF₃, halo, or —O(CH₂)ₙ—Rₓ, where n is 2-4 and Rₓ is N-methylpiperazine, morpholine or 2-methylpyrrolidine.

In a more specific aspect of structure (I) above, L₂ is —C(═S)NH— or —C(═S)NHCH₂—.

In a more specific aspect of structure (I) above, w is —S(═O)₂NHC(═O)CH₃, —NHC(═O)R_{y}, —C(═O)NH—, —NHS(═O)₂R_{y}, —S(═O)₂NHR_{y}— where R_{y} is alkyl, substituted alkyl, cycloalkyl, substituted alkyl, carbocycle, substituted carbocycle, heterocycle, substituted heterocycle, —NH₂, —NH₂.HCl, and —S(═O)₂—R_{z}, where R_{z} is selected from alkyl, substituted alkyl, amine, N-methylpiperazine, morpholine, 2-methylpyrrolidine.

In a more specific aspect of structure (I) above, w is —S(═O)₂NHC(═O)CH₃ or —S(═O)₂—R_{z}, where R_{z} is selected from C₁-C₃ alkyl, C₁-C₃ substituted alkyl or amine.

In a more specific aspect of structure (I) above, w is —S(═O)₂NHC(═O)CH₃, —S(═O)₂NH₂ or —S(═O)₂CH₃.

In a more specific aspect of structure (I) above, w is —S(═O)₂NHC(═O)CH₃.

In a more specific aspect of structure (I) above, R₁, R₂ and R₃ are selected from hydrogen, —NH₂, —OCH₃, —OH, —CF₃, halo, or —O(CH₂)ₙ—Rₓ, where n is 2-4 and Rₓ is N-methylpiperazine, morpholine or 2-methylpyrrolidine, and w is —S(═O)₂NHC(═O)CH₃, —S(═O)₂NH₂ or —S(═O)₂CH₃.

In a more specific aspect of structure (I) above, R₁ and R₂ are selected from hydrogen, halo, —CF₃ or —OH, R₃ is hydrogen, and w is —S(═O)₂NHC(═O)CH₃, —S(═O)₂NH₂ or —S(═O)₂CH₃.

In a more specific aspect of structure (I) above, X is NH, Z is CH, L₂ is —C(═S)NH—, and the compound has the following structure (II):

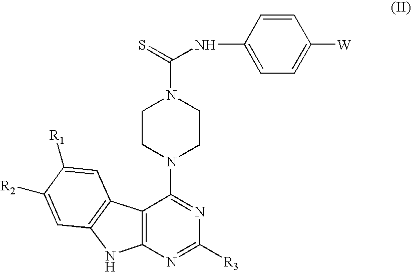

In a more specific aspect of structure (II) above, R₁ and R₂ are selected from —OCH₃, —OH, —CF₃, halo, or —O(CH₂)ₙ—Rₓ, where n is 2-4 and Rₓ is N-methylpiperazine, morpholine or 2-methylpyrrolidine, and R₃ is selected from hydrogen or —NH₂.

In a more specific aspect of structure (II) above, R₁ and R₂ are selected from —OCH₃, —OH, —CF₃ or halo, and R₃ is hydrogen.

In a more specific aspect of structure (II) above, w is —S(═O)₂NHC(═O)CH₃ or —S(═O)₂—R_{z}, where R_{z} is selected from C₁-C₃ alkyl, C₁-C₃ substituted alkyl or amine.

In a more specific aspect of structure (II) above, w is —S(═O)₂NHC(═O)CH₃, —S(═O)₂NH₂ or —S(═O)₂CH₃.

In a more specific aspect of structure (II) above, R₁ and R₂ are selected from —OCH₃, —OH, —CF₃ or halo, R₃ is hydrogen, and w is —S(═O)₂NHC(═O)CH₃, —S(═O)₂NH₂ or —S(═O)₂CH₃.

In a more specific aspect of structure (II) above, R₁ and R₂ are selected from —OCH₃, —OH, —CF₃ or halo, R₃ is hydrogen, and w is —S(═O)₂NHC(═O)CH₃, —S(═O)₂NH₂ or —S(═O)₂CH₃.

In a more specific aspect of structure (II) above, R₁ and R₂ are methoxy, R₃ is hydrogen, w is —S(═O)₂NHC(═O)CH₃, and the compound has the following structure (III):

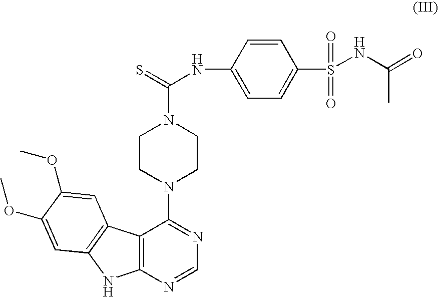

In a more specific aspect of structure (II) above, R₁ is —Cl, R₂ is —CF₃, R₃ is hydrogen, w is —S(═O)₂NHC(═O)CH₃, and the compound has the following structure (IV):

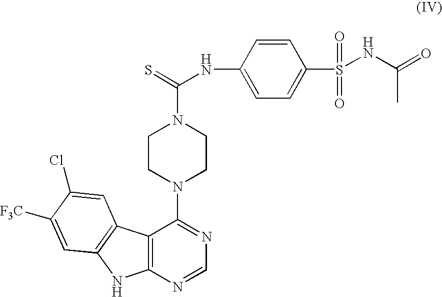

In more specific aspects of structure (I) above, compounds are provided having structures set forth in Table 1 below.

TABLE 1

| | Structure |
|---|---|
| 1-1 | 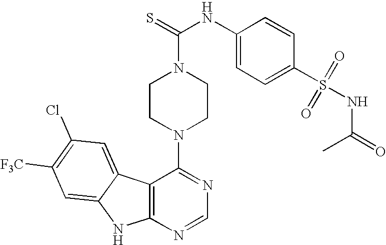 |
| 1-2 | 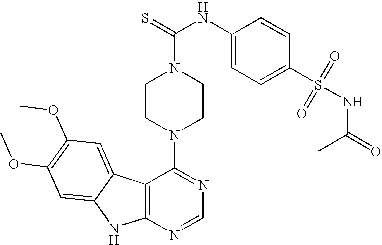 |
| 1-3 | 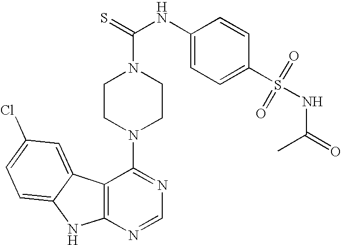 |
| 1-4 | 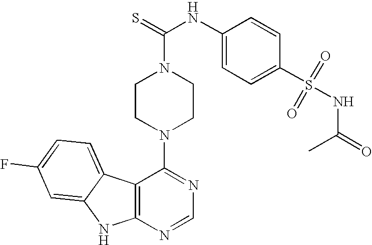 |
| 1-5 | 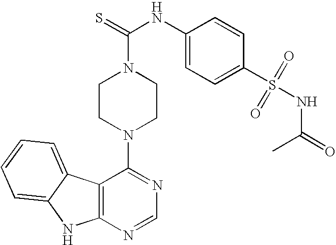 |
| 1-6 | 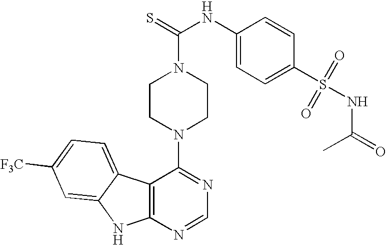 |
| 1-7 | 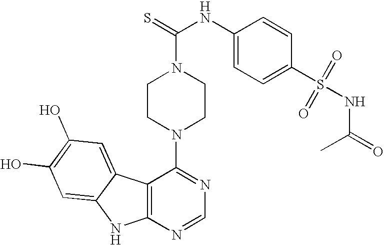 |
| 1-8 | 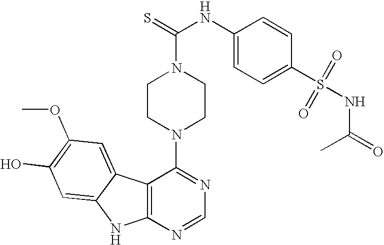 |
| 1-9 | 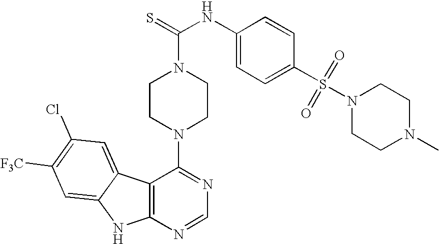 |
| 1-10 | 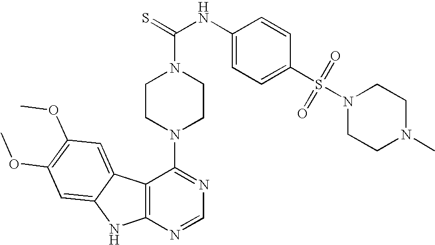 |
| 1-11 | 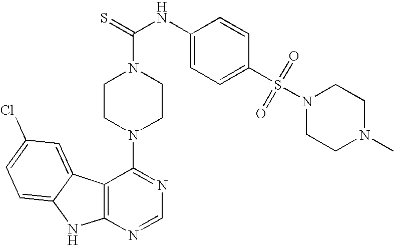 |
| 1-12 | 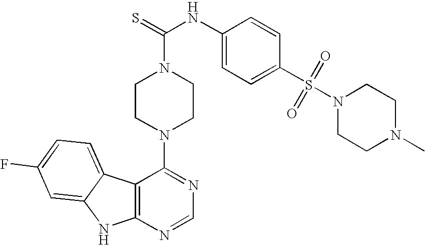 |
| 1-13 | 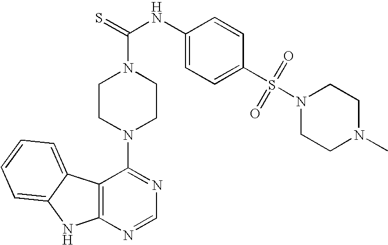 |
| 1-14 | 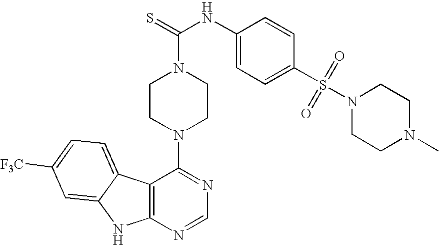 |
| 1-15 | 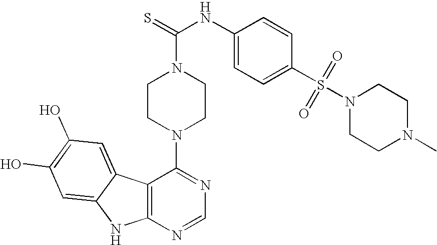 |
| 1-16 | 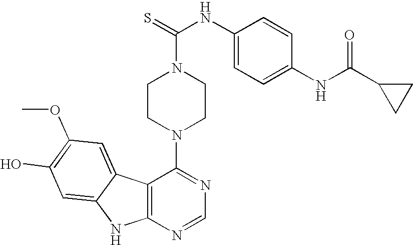 |
| 1-17 | 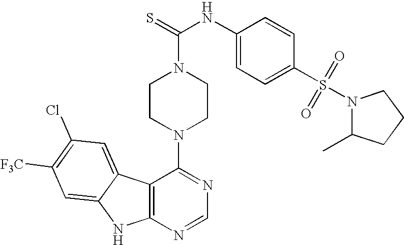 |
| 1-18 | 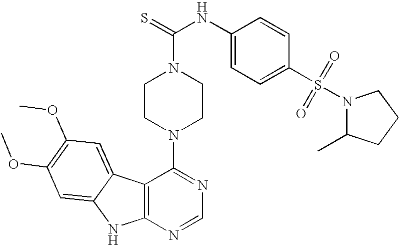 |
| 1-19 | 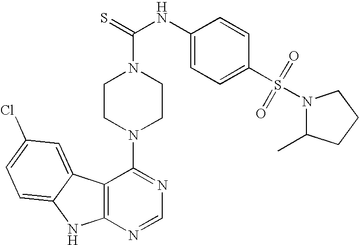 |
| 1-20 | 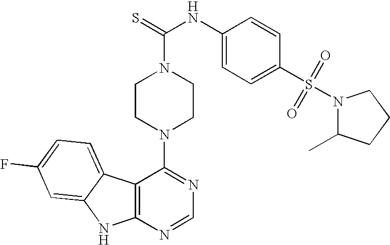 |
| 1-21 | 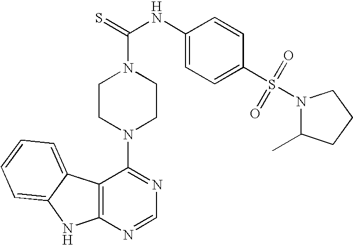 |
| 1-22 | 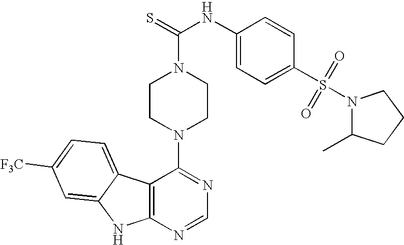 |
| 1-23 | 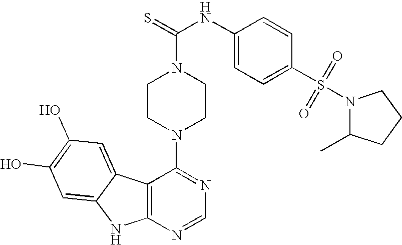 |
| 1-24 | 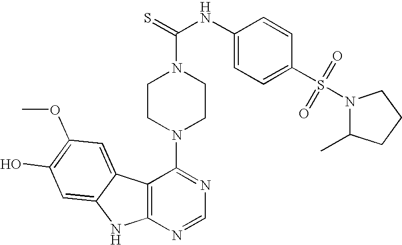 |
| 1-25 | 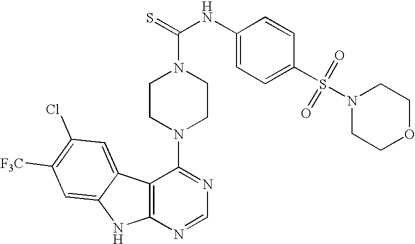 |
| 1-26 | 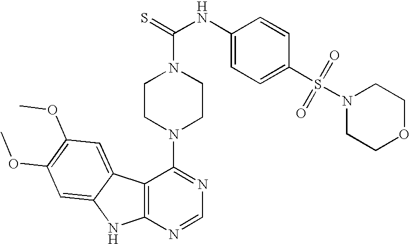 |
| 1-27 | 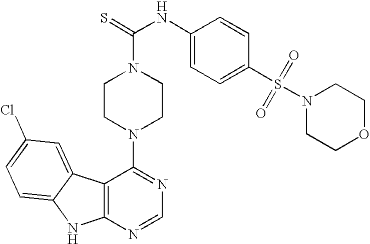 |
| 1-28 | 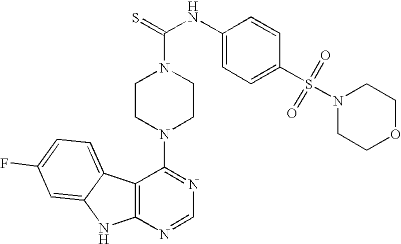 |
| 1-29 | 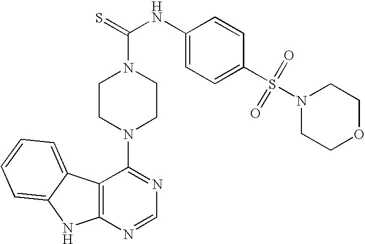 |
| 1-30 | 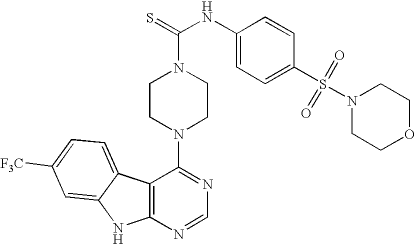 |
| 1-31 | 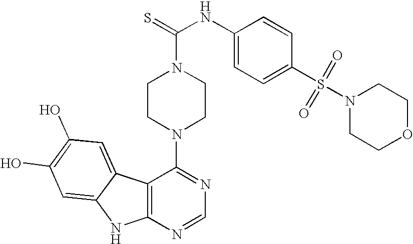 |
| 1-32 | 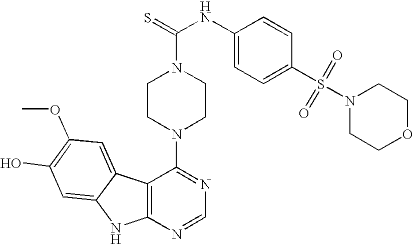 |
| 1-33 | 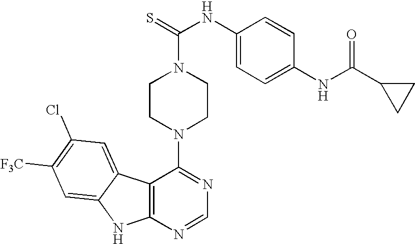 |
| 1-34 | 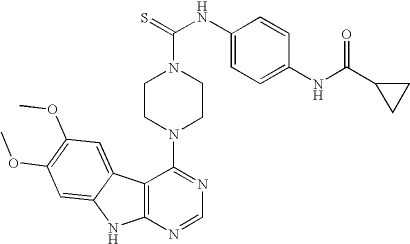 |
| 1-35 | 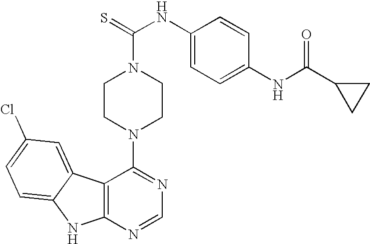 |
| 1-36 | 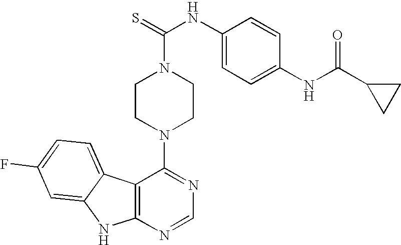 |
| 1-37 | 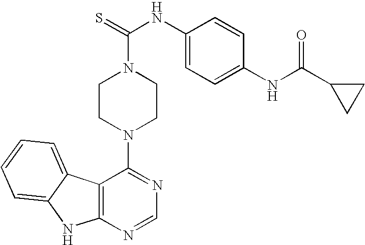 |
| 1-38 | 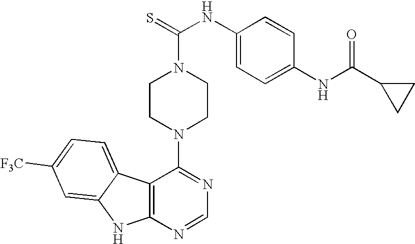 |
| 1-39 | 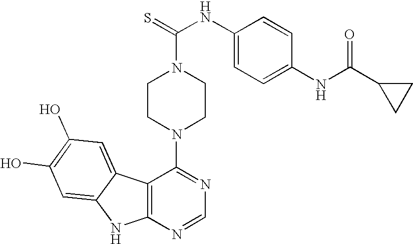 |
| 1-40 | 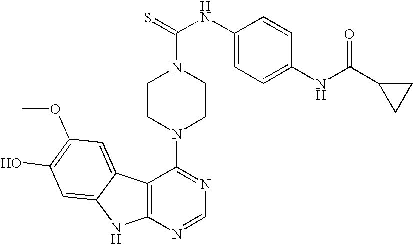 |
| 1-41 | 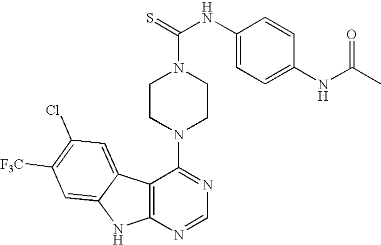 |
| 1-42 | 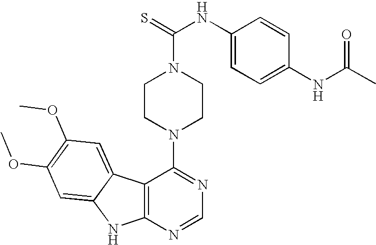 |
| 1-43 | 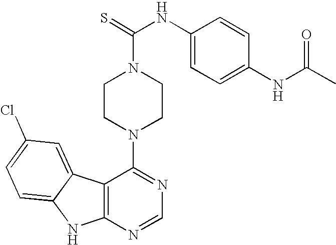 |
| 1-44 | 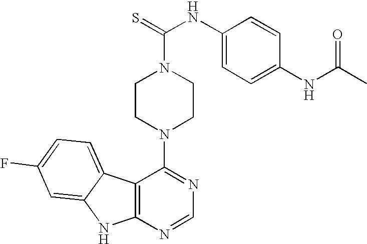 |
| 1-45 | 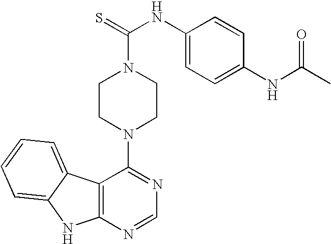 |
| 1-46 | 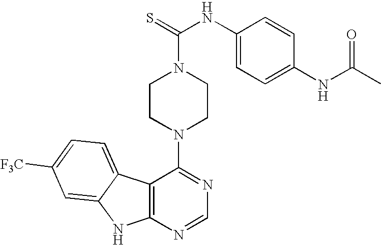 |
| 1-47 | 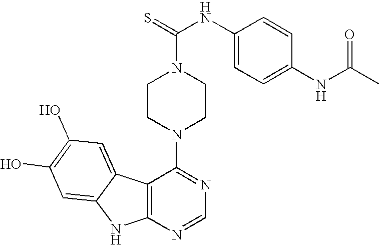 |
| 1-48 | 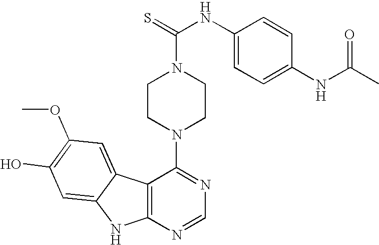 |
| 1-49 | 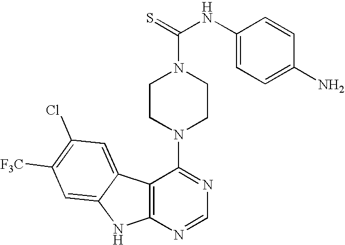 |
| 1-50 | 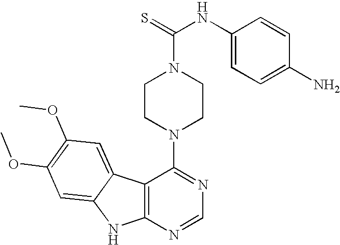 |
| 1-51 | 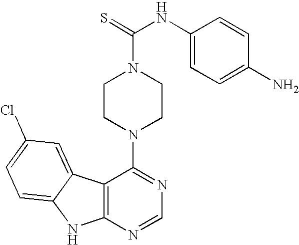 |
| 1-52 | 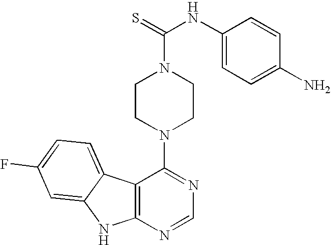 |
| 1-53 | 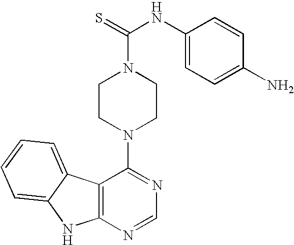 |
| 1-54 | 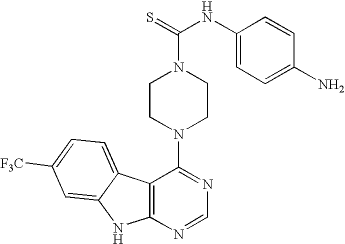 |
| 1-55 | 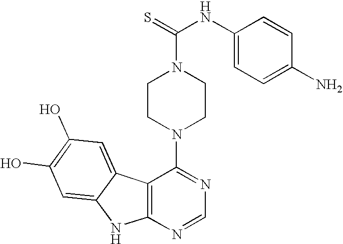 |
| 1-56 | 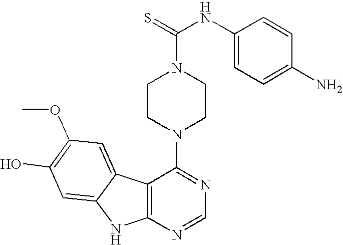 |
| 1-57 | 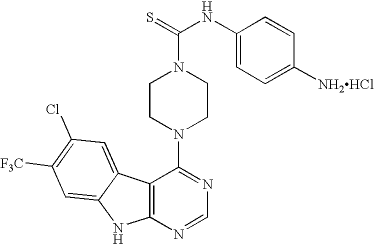 |
| 1-58 | 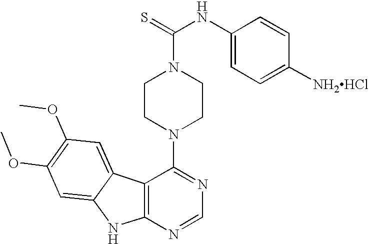 |
| 1-59 | 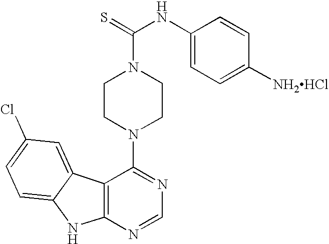 |
| 1-60 | 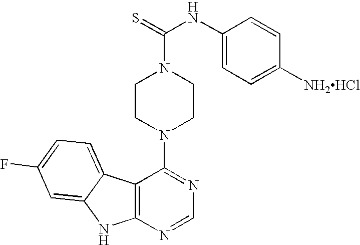 |
| 1-61 | 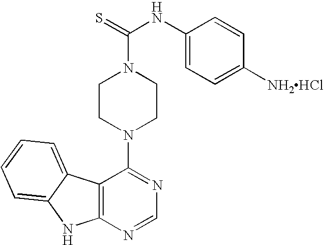 |
| 1-62 | 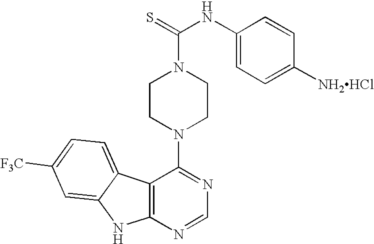 |
| 1-63 | 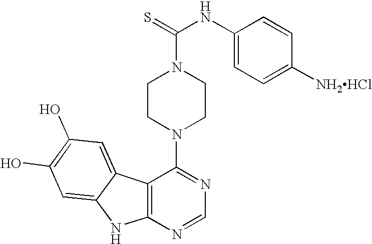 |
| 1-64 | 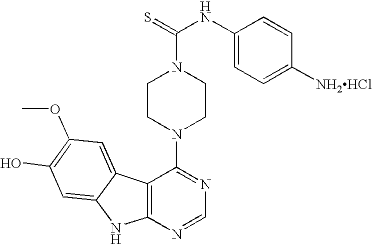 |
| 1-65 | 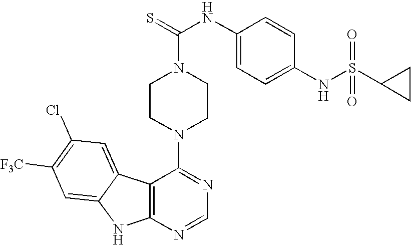 |
| 1-66 | 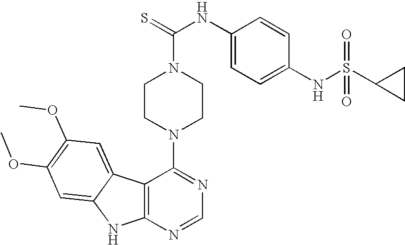 |
| 1-67 | 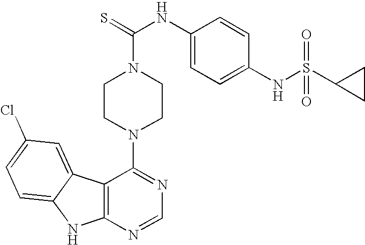 |
| 1-68 | 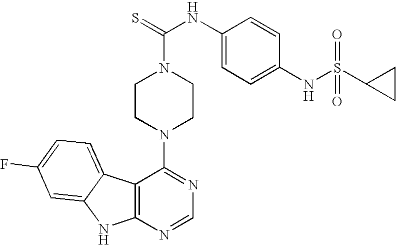 |
| 1-69 | 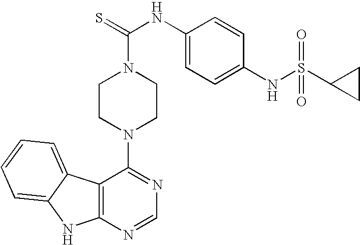 |
| 1-70 | 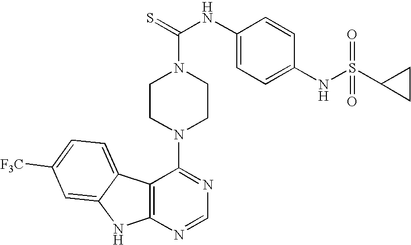 |
| 1-71 | 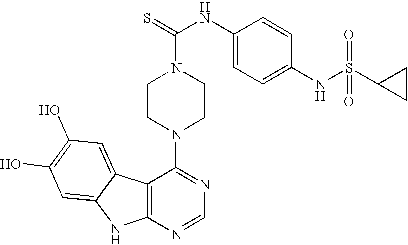 |
| 1-72 | 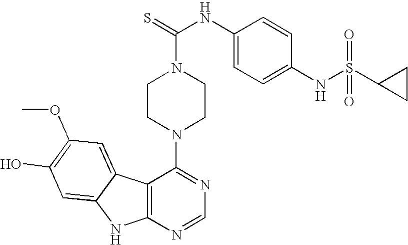 |
| 1-73 | 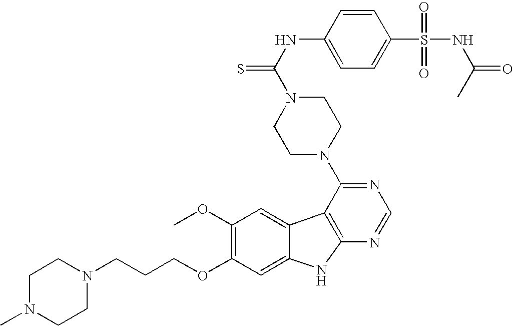 |
| 1-74 | 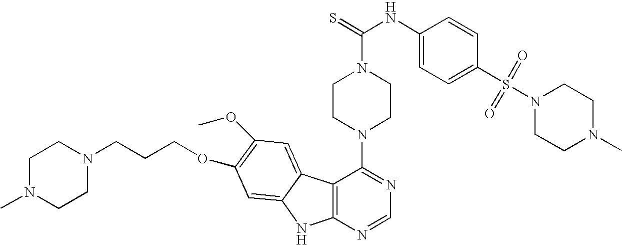 |
| 1-75 | 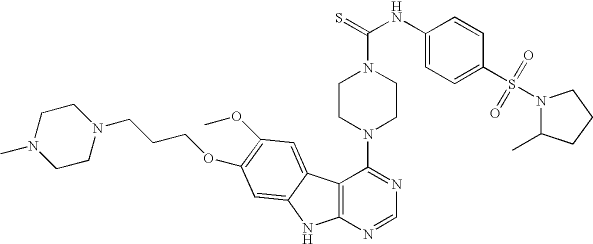 |
| 1-76 | 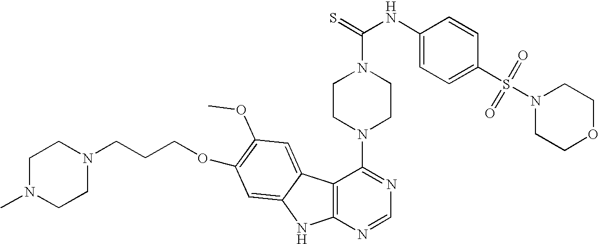 |
| 1-77 | 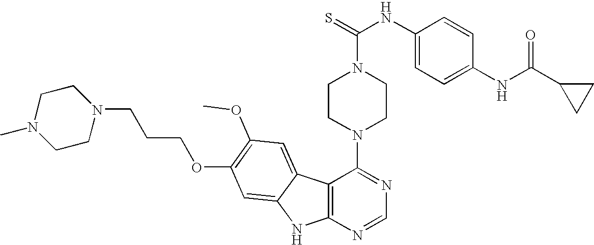 |
| 1-78 | 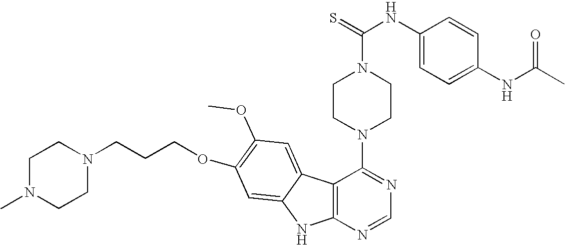 |
| 1-79 | 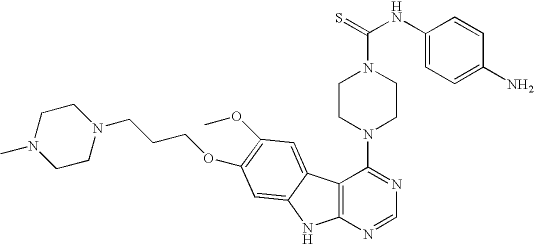 |
| 1-80 | 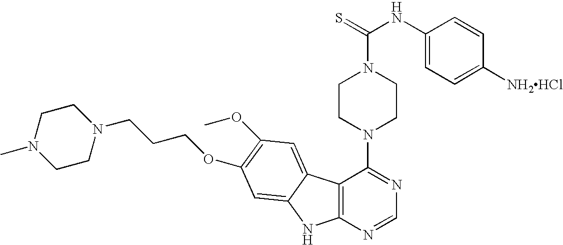 |
| 1-81 | 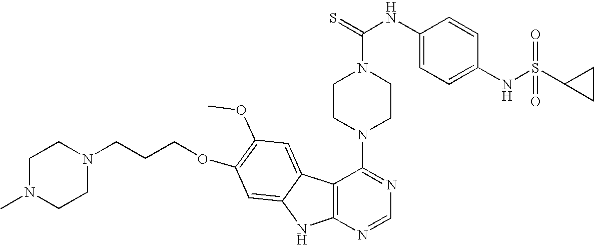 |
| 1-82 | 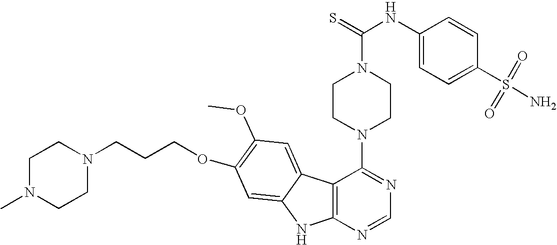 |
| 1-83 | 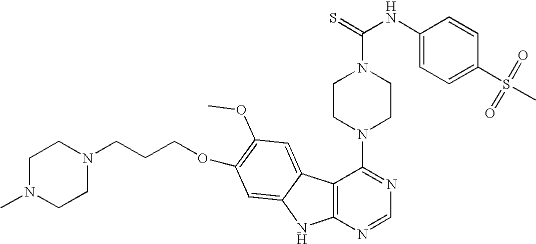 |
| 1-84 | 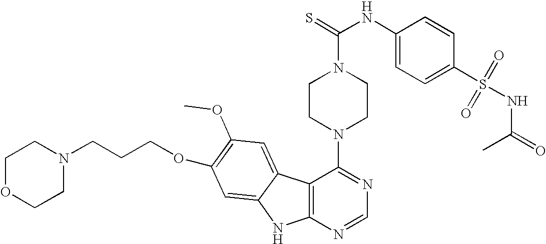 |
| 1-85 | 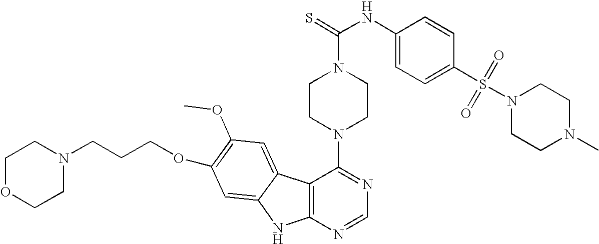 |
| 1-86 | 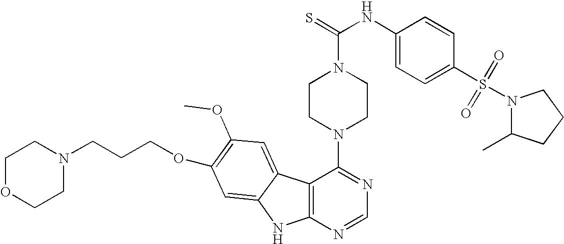 |
| 1-87 | 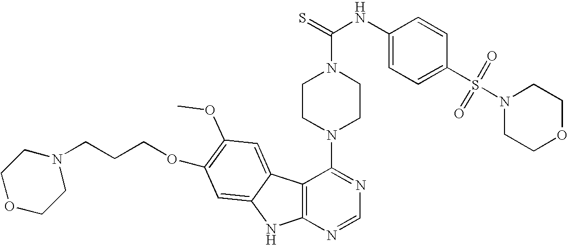 |
| 1-88 | 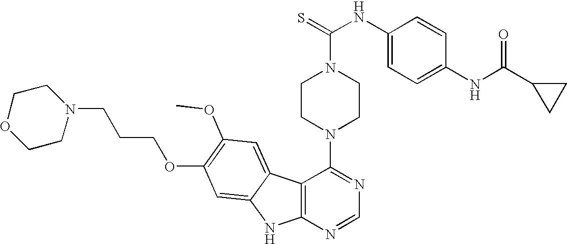 |
| 1-89 | 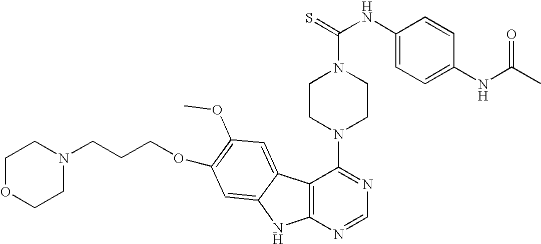 |
| 1-90 | 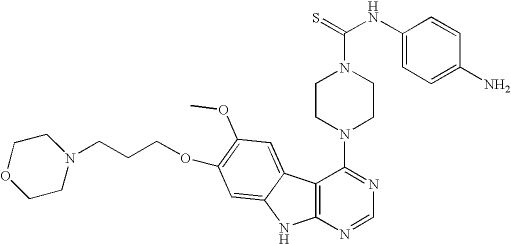 |
| 1-91 | 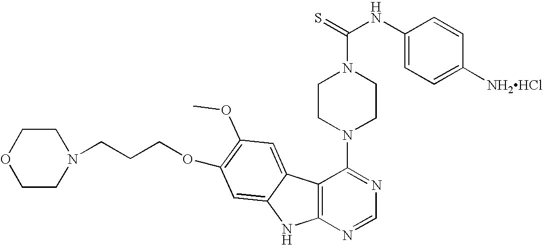 |
| 1-92 | 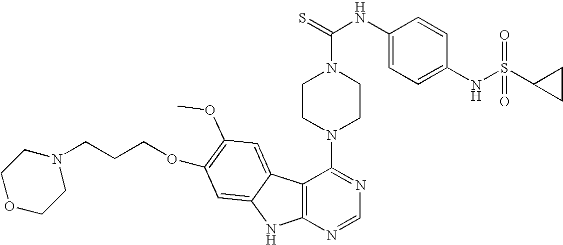 |
| 1-93 | 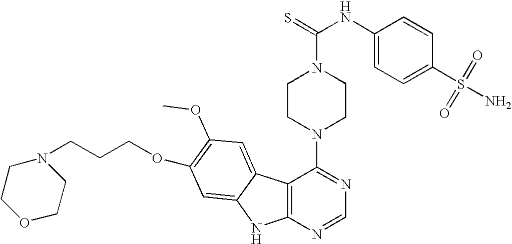 |
| 1-94 | 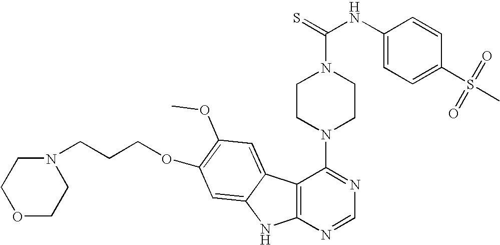 |
| 1-95 | 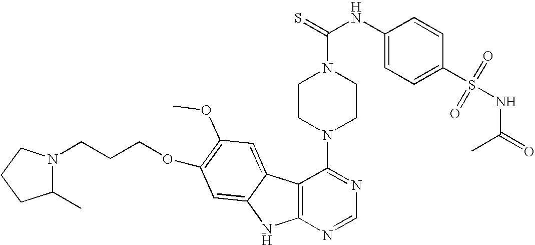 |
| 1-96 | 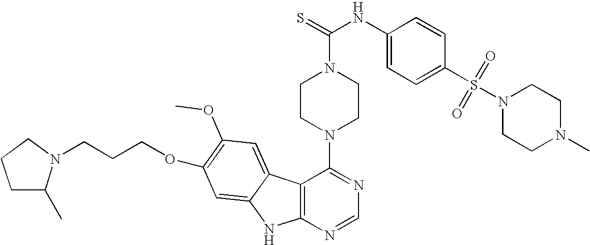 |
| 1-97 | 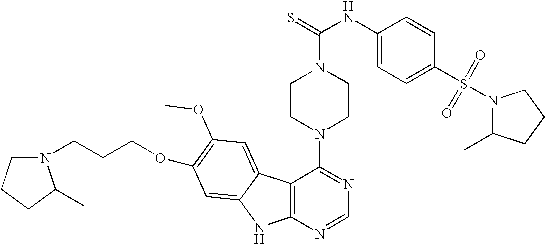 |
| 1-98 | 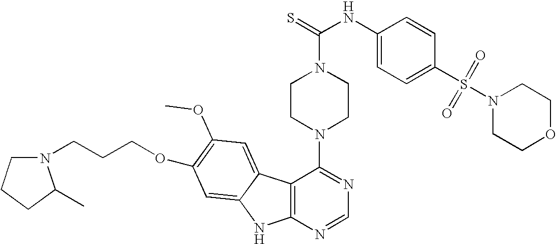 |
| 1-99 | 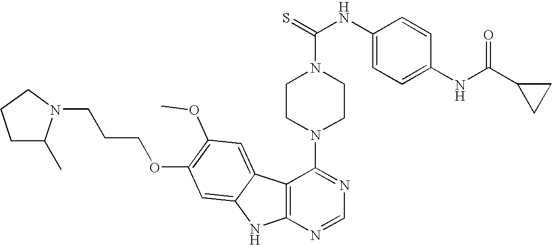 |
| 1-100 | 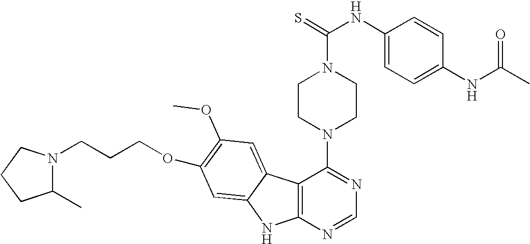 |
| 1-101 | 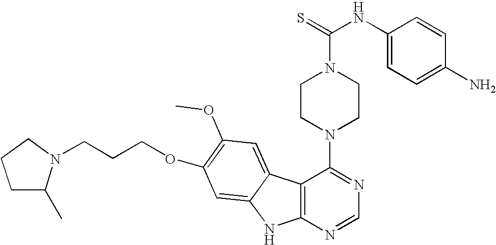 |
| 1-102 | 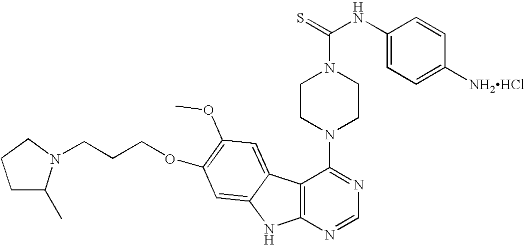 |
| 1-103 | 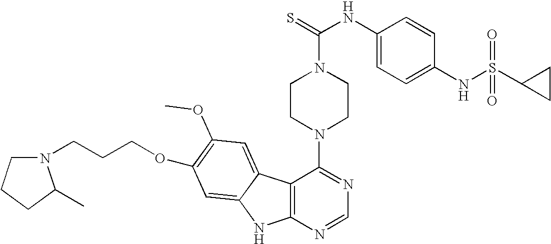 |
| 1-104 | 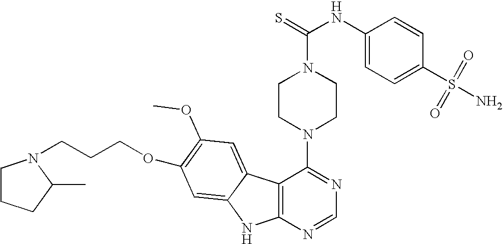 |
| 1-105 | 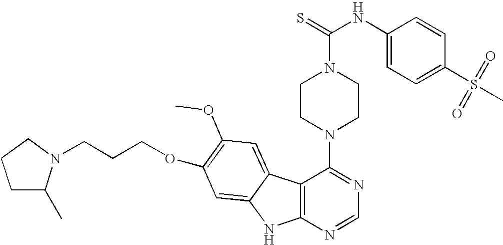 |
| 1-106 | 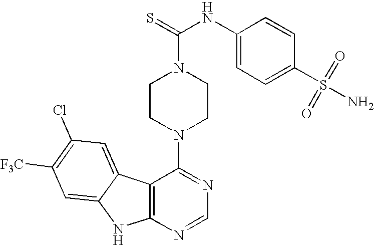 |
| 1-107 | 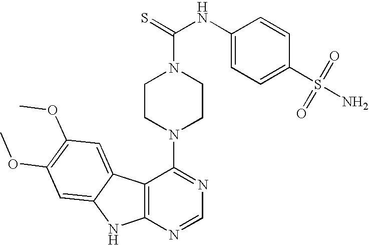 |
| 1-108 | 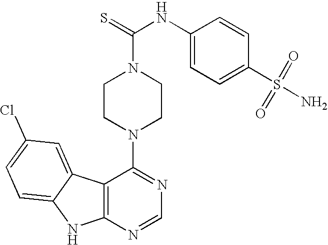 |
| 1-109 | 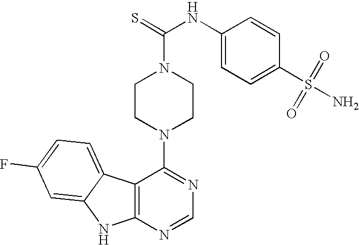 |
| 1-110 | 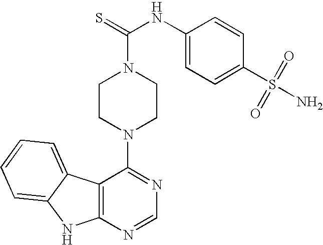 |
| 1-111 | 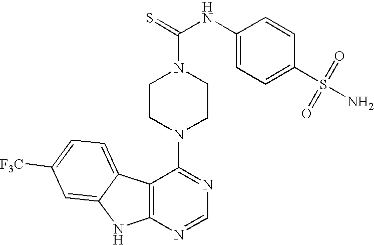 |
| 1-112 | 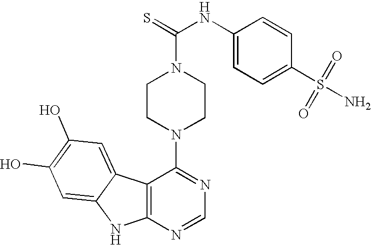 |
| 1-113 | 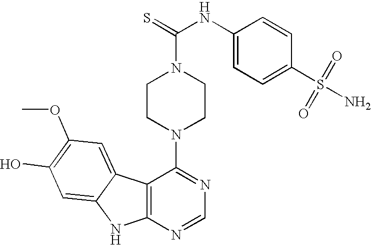 |
| 1-114 | 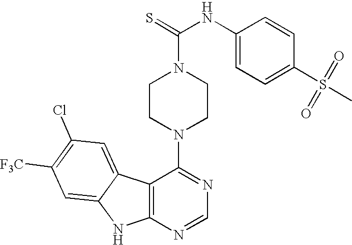 |
| 1-115 | 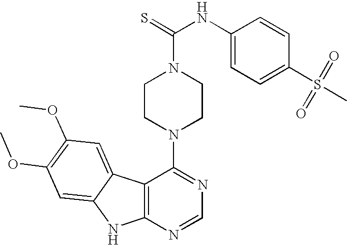 |
| 1-116 | 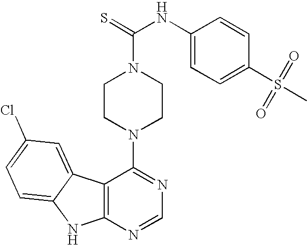 |
| 1-117 | 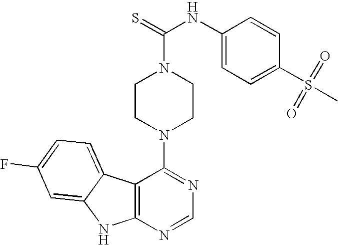 |
| 1-118 | 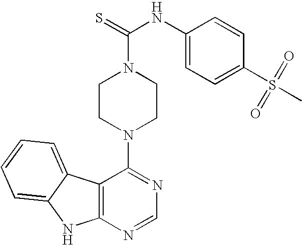 |
| 1-119 | 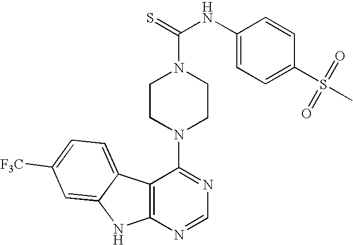 |
| 1-120 | 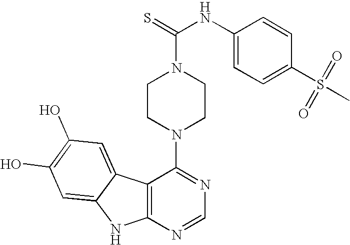 |
| 1-121 | 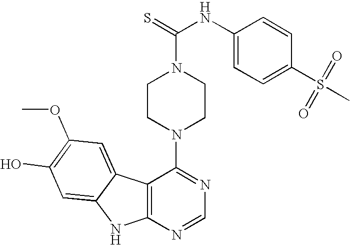 |

In another aspect, as described in Examples 21-53 below, additional compounds of the invention were identified as a result of further structural and functional analyses. These studies identified highly active compounds of structure (I) where R₁, R₂, R₃, X, Z and L₂ are as defined above and where w is —S(═O)₂NHR_{y}, where R_{y} is alkyl, substituted alkyl, cycloalkyl, substituted cycloalkyl, carbocycle, substituted carbocycle, heterocycle or substituted heterocycle.

In a more specific aspect of these structures, w is —S(═O)₂NHR_{y}, where R_{y} is carbocycle, substituted carbocycle, heterocycle or substituted heterocycle.

In a more specific aspect of these structures, w is —S(═O)₂NHR_{y}, where R_{y} is

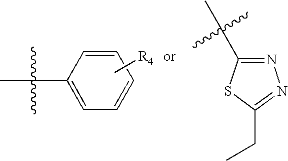

where R₄ is hydrogen, alkyl, halo or haloalkyl.

In a more specific aspect of these structures, w is —S(═O)₂NHR_{y}, where R_{y} is

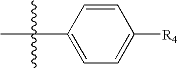

where R₄ is hydrogen, alkyl, halo or haloalkyl.

In a more specific aspect of these structures, X is NH and Z is CH.

In a more specific aspect, L₂ is —C(═S)NH—.

In a more specific aspect, R₁ and R₂ are selected from hydrogen, —NH₂, —OR, —OH, halo, haloalkyl or morpholine, where R is C₁-C₃ alkyl.

In a more specific aspect, R₃ is hydrogen.

In a more specific aspect, R₁ and R₂ are selected from —OCH₃, halo and morpholine, R₃ is hydrogen, and w is —S(═O)₂NHR_{y}, where R_{y} is:

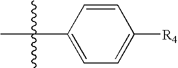

where R₄ is alkyl, halo or haloalkyl.

In a more specific aspect, X is NH, Z is CH, L₂ is —C(═S)NH—, and the compound has the following structure (V):

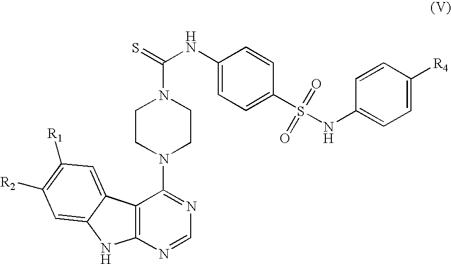

where R₄ is alkyl, halo or haloalkyl.

In a more specific aspect of structure (V), R₁ and R₂ are selected from hydrogen, —NH₂, —OR, —OH, halo, haloalkyl or morpholine, where R is C₁-C₃ alkyl.

In a more specific aspect of structure (V), R₁ and R₂ are selected from —OR, halo or morpholine, where R is C₁-C₃ alkyl.

In a more specific aspect of structure (V), R₄ is selected from C₁-C₃ alkyl, halo or haloalkyl.

In a more specific aspect of structure (V), R₁ and R₂ are selected from —OR, halo or morpholine, where R is C₁-C₃ alkyl, and R₄ is selected from C₁-C₃ alkyl, halo or haloalkyl.

In another aspect of structure (I) above, X is NH, Z is CH, L₂ is —C(═S)NH—, R₃ is hydrogen, and the compound has the following structure (VI):

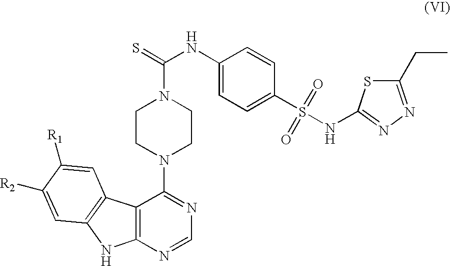

where R₁ and R₂ are selected from hydrogen, —OR, —OH, halo, haloalkyl or morpholine, where R is C₁-C₃ alkyl.

In more specific aspects of structure (V) and (VI) above, compounds are provided having structures set forth in the following Table 2 below.

TABLE 2

| | Structure |
|---|---|
| 2-1 | 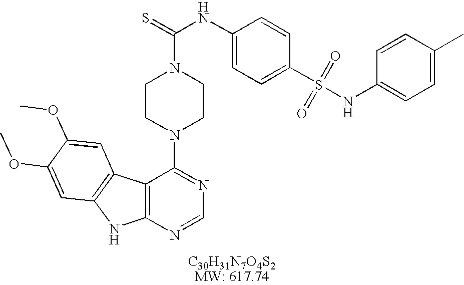 |
| 2-2 | 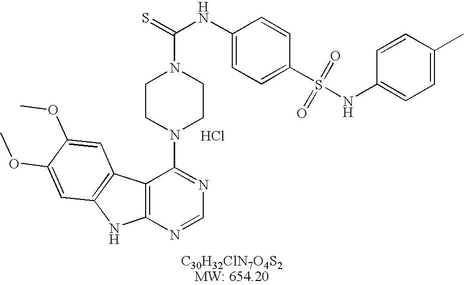 |
| 2-3 | 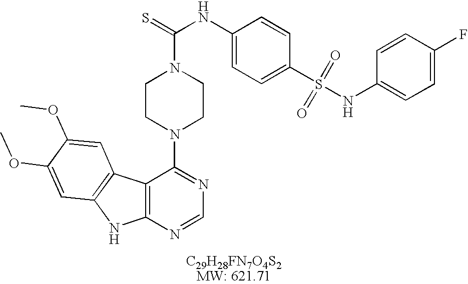 |
| 2-4 | 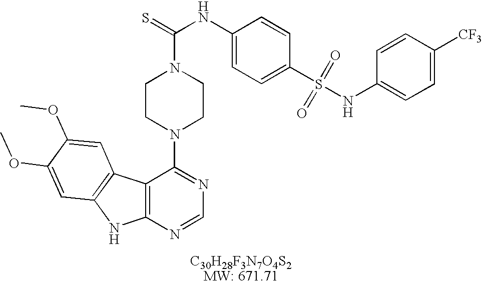 |
| 2-5 | 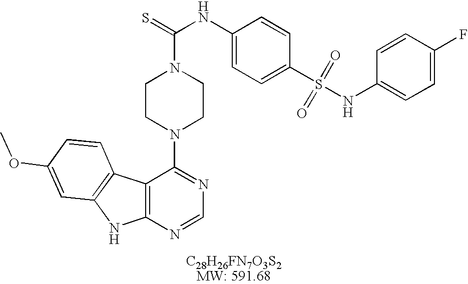 |
| 2-6 | 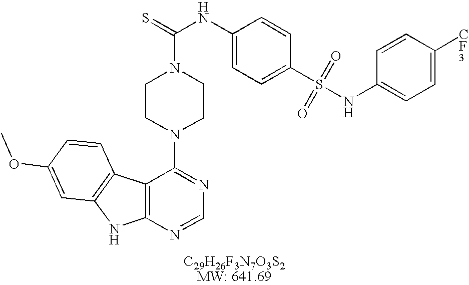 |
| 2-7 | 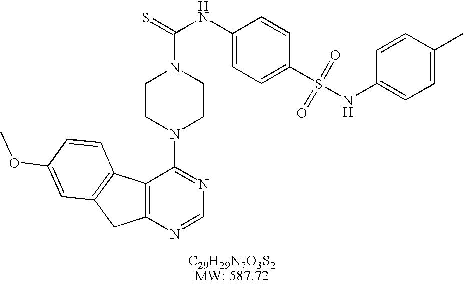 |
| 2-8 | 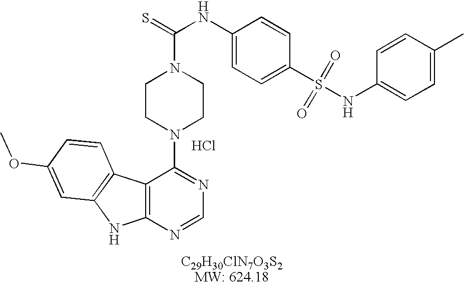 |
| 2-9 | 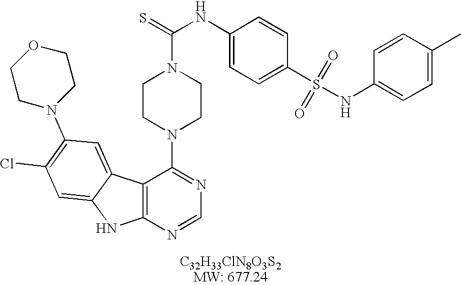 |
| 2-10 | 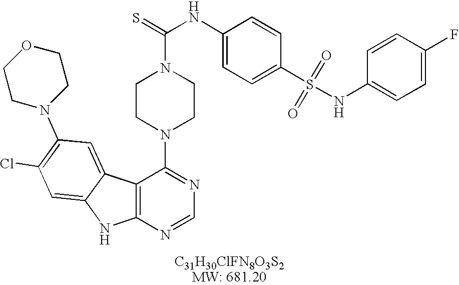 |
| 2-11 | 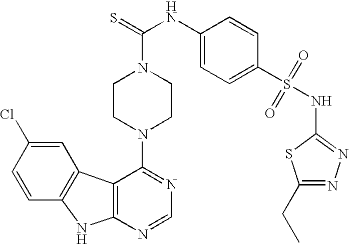 |
| 2-12 | 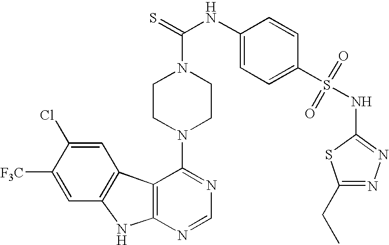 |
| 2-13 | 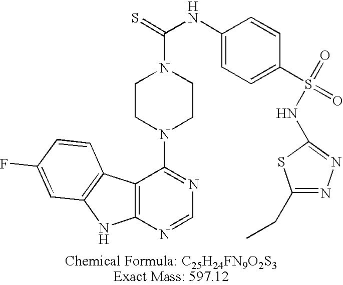 |
| 2-14 | 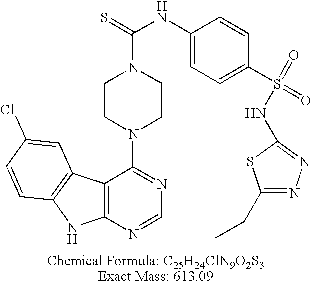 |
| 2-15 | 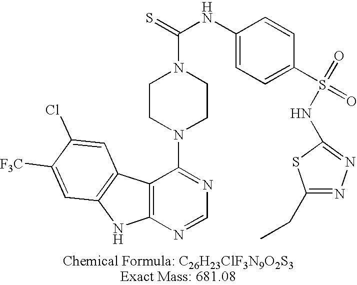 |

In a more specific aspect of structure (I) above, X is NH, Z is CH, L₂ is —C(═S)NH—, R₃ is hydrogen, and the compound has the following structure (VII):

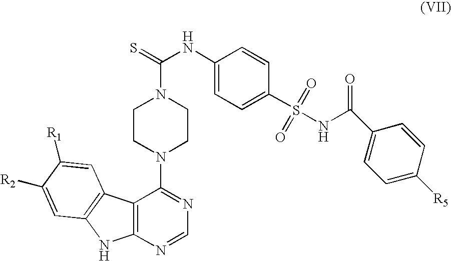

In a more specific aspect of structure (VII) above, R₁ and R₂ are selected from hydrogen, —OR, —OH, halo, haloalkyl or morpholine, where R is C₁-C₃ alkyl; and where R₅ hydrogen, alkyl, alkoxy, haloalkyl, halo, hydroxyl, or substituted alkoxy.

In a more specific aspect of structure (VII) above, the compound has the following structure:

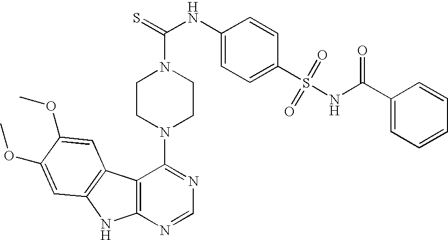

Further illustrative compounds according to these aspects are provided having structures set forth in Table 3 below.

TABLE 3

| | Structure |
|---|---|
| 3-1 | 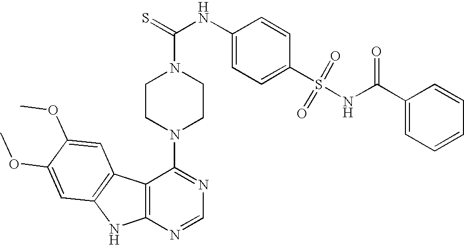 |
| 3-2 | 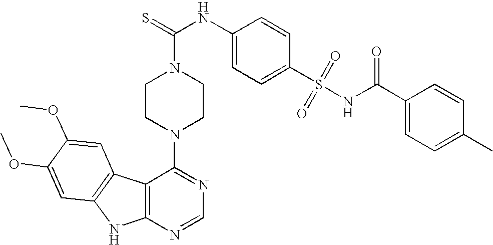 |
| 3-3 | 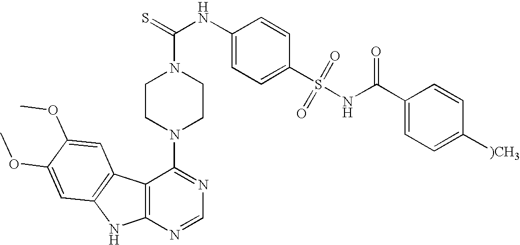 |
| 3-4 | 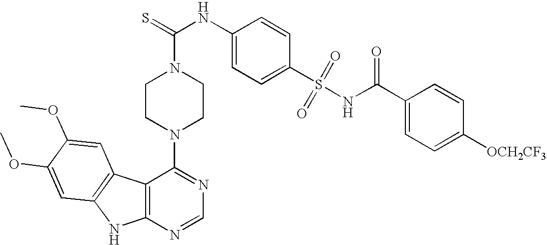 |
| 3-5 | 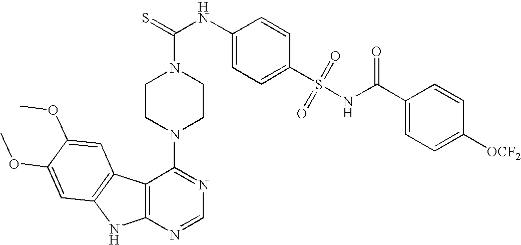 |
| 3-6 | 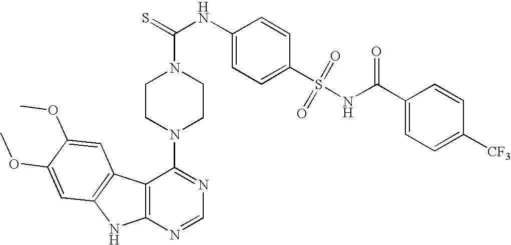 |
| 3-7 | 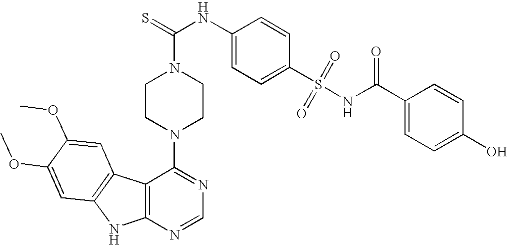 |
| 3-8 | 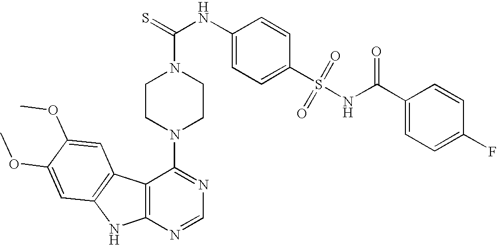 |
| 3-9 | 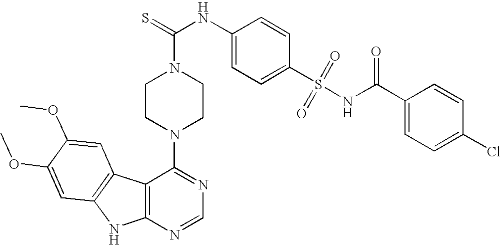 |

Compounds that have the same molecular formula but differ in the nature or sequence of bonding of their atoms or the arrangement of their atoms in space are termed “isomers”. Isomers that differ in the arrangement of their atoms in space are termed “stereoisomers”. Stereoisomers that are not mirror images of one another are termed “diastereomers” and those that are non-superimposable mirror images of each other are termed “enantiomers”. When a compound has an asymmetric center, for example, it is bonded to four different groups, a pair of enantiomers is possible. An enantiomer can be characterized by the absolute configuration of its asymmetric center and is described by the R— and S— sequencing rules of Cahn and Prelog (Cahn, R., Ingold, C., and Prelog, V. Angew. Chem. 78:413-47, 1966; Angew. Chem. Internat. Ed. Eng. 5:385-415, 511, 1966), or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture”.

The compounds of this invention may possess one or more asymmetric centers; such compounds can therefore be produced as individual (R)- or (S)-stereoisomers or as mixtures thereof. Unless indicated otherwise, the description or naming of a particular compound in the specification and claims is intended to include both individual enantiomers and mixtures, racemic or otherwise, thereof. The methods for the determination of stereochemistry and the separation of stereoisomers are well-known in the art (see discussion in Ch. 4 of ADVANCED ORGANIC CHEMISTRY, 4^{th} edition, March, J., John Wiley and Sons, New York City, 1992).

The compounds of the present invention may exhibit the phenomena of tautomerism and structural isomerism. For example, the compounds described herein may adopt an E or a Z configuration about the double bond connecting the 2-indolinone moiety to the pyrrole moiety or they may be a mixture of E and Z. This invention encompasses any tautomeric or structural isomeric form and mixtures thereof which possess the ability to modulate aurora-2 kinase activity and is not limited to, any one tautomeric or structural isomeric form.

It is contemplated that a compound of the present invention would be metabolized by enzymes in the body of the organism such as human being to generate a metabolite that can modulate the activity of the protein kinases. Such metabolites are within the scope of the present invention.

Certain compounds of the invention may be made by one skilled in this field according using known methodologies, as illustratively set forth in the following general reaction schemes 1-3 below, as well as by the more detailed procedures set forth in the Examples 1-16 below.

Chlorination of (un)substituted 6-membered aromatic moieties can be carried out in the presence of sulfuryl chloride at about 0° C. The 4-chloro-(un)substituted benzene (2) can be nitrated to obtain 1-chloro-(un)substituted-2-nitrobenzene (3) with fuming nitric acid, preferably without the temperature exceeding about 25° C. Ethyl 2-cyano-2-(un)substituted-2-nitrophenyl)acetate (4) can be prepared by reacting compound 3 with ethylcyanoacetate in the presence of potassium-tert-butoxide in THF (yielded compound 4 at 23%). Further the yields can be optimized at this stage by reacting compound 3 in the presence of K₂CO₃ in DMF at a temperature of about 155° C. for 6 hours to give the ethylcyano ester in high yield. Reduction of ester 4, can be carried out with excess of Zn dust (4-6 eq) using known conditions to give an ethyl 2-amino-5,6-dimethoxy-1H-indole-3-carboxylate (5) without an N-hydroxy side product.

Cyclization of ethyl 2-amino-5,6-dimethoxy-1H-indole-3-carboxylate (5) to the corresponding dihydro-4H-pyrimido[4,5-b]indoles, can be performed by heating at about 200-220° C. in formamide and catalytic sodium methoxide. The dihydro-pyrimidines can be converted to 4-chlorides (6) in good yields with thionylchloride and/or POCl₃ in dioxane solvent. The 4-chlorides can be utilized in preparing 4-piprazine substituted tricyclic analogues as outlined in Scheme 1. The 4-chlorides can be reacted with piprazine in the presence of pyridine in dioxane solvent at reflux temperature to give compound 8 in good yields. The substitutent at the R₃ position can be obtained by reacting either cyclic ethyl esters in presence of cyanoacetamide and dry HCl to give the guanidine analogues 10. These compounds can be cyclized to 3-substituted tricyclic dihydro-pyrimidine in presence of aqueous NaOH.

Certain intermediates that can be utilized in the preparation of target compounds are outlined in Scheme 2 and detailed in Scheme 3. The variously substituted aromatic amines can be treated with thiophosgene in dichloromethane in presence of CaCO₃ and water to give isothiocyanate analogue 13 in high yields. The compounds of formula I having 4-substituted piprazine analogues can be prepared by reacting compound 13 in the presence of pyridine and dioxane solvent. Compound 14 on treatment with 1-bromo-3-chloropropane and cesium carbonate in acetonitrile yielded the 1-(3-chloropropoxy)-4-chloro-2-methoxybenzene 15. Various carbocyclic compounds such as N-methylpiperazine, morpholine and or 2-methylpyrrolidine were reacted with compound 15 in Acetonitrile gave the compound 17 in high yields (Scheme 2). Subsequently it was nitrated and under similar conditions the Ethyl 2-cyano-2-(un)substituted-2-nitrophenyl)acetates were prepared as described for the preparation of compound 4 shown in Scheme 1.

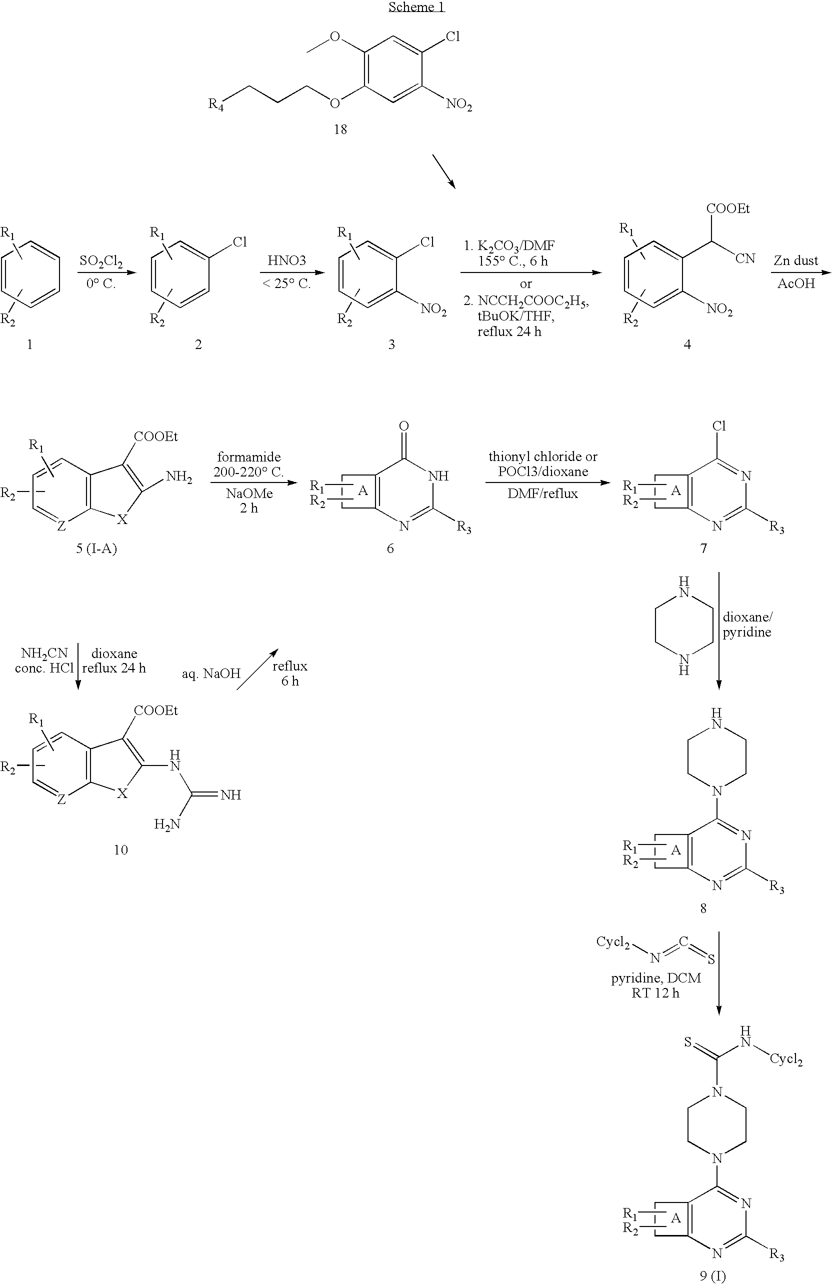

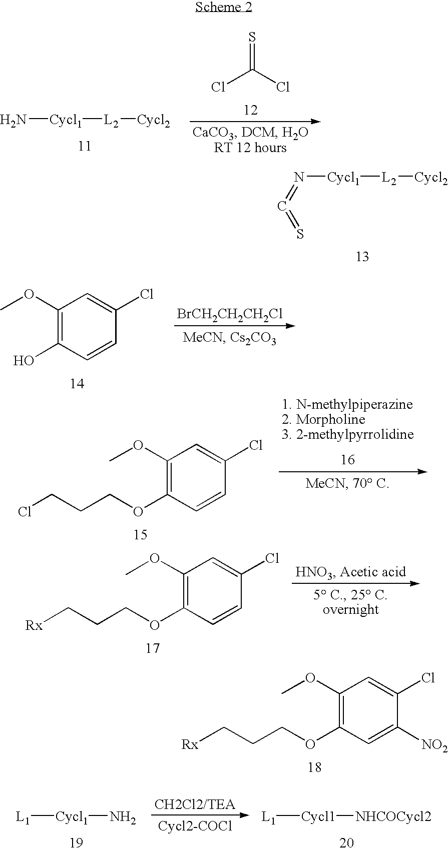

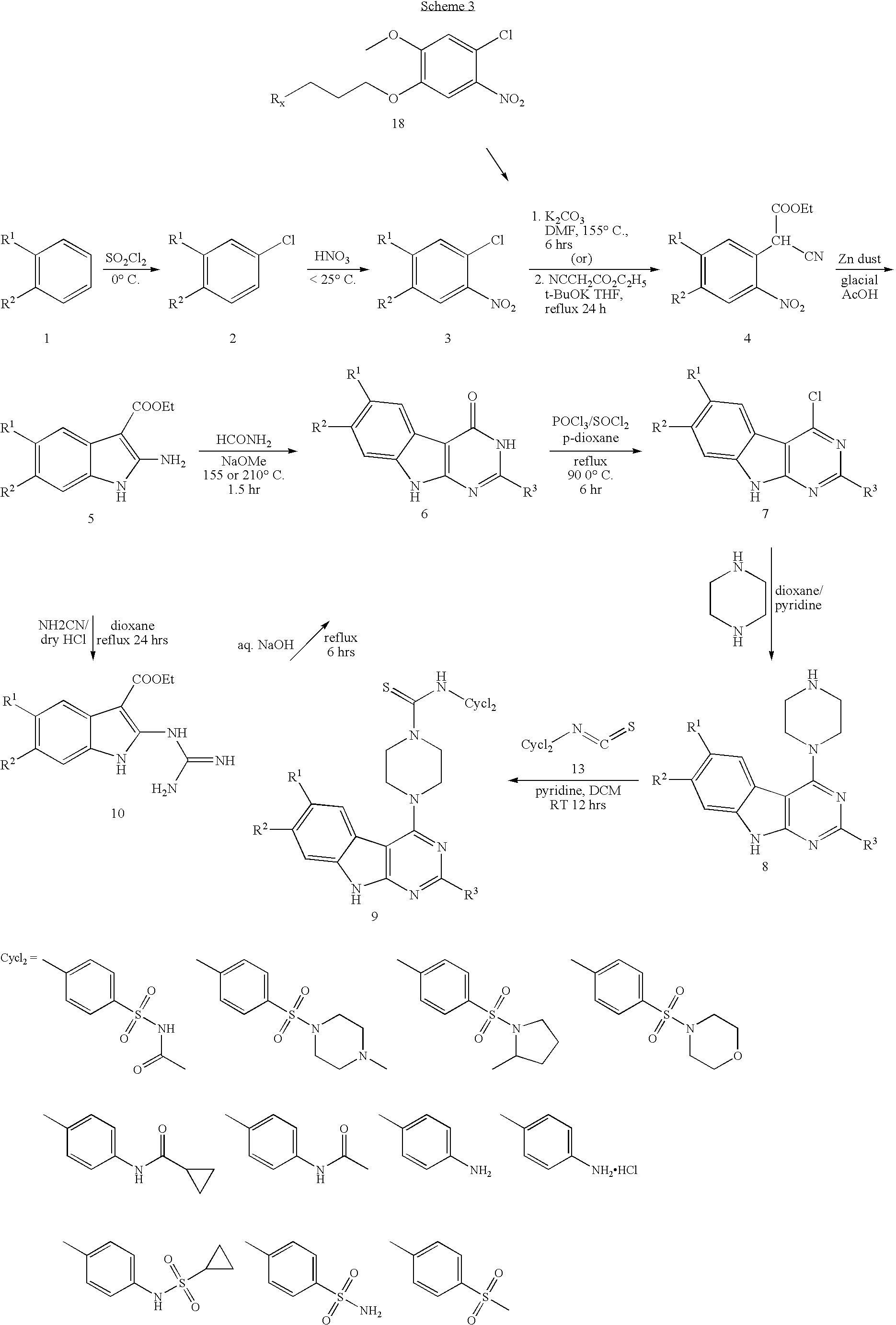

Additional compounds of the invention, including those set forth in Structure (V) above, may be made by one skilled in this field using known methodologies, as illustratively set forth in the following general reaction schemes 4-6 below, as well as by the more detailed procedures set forth in the Examples 21-53 below.

Chlorination of (un)substituted 6-membered aromatic moieties can be carried out in the presence of sulfuryl chloride at about 0° C. The 4-chloro-(un)substituted benzene 2 can be nitrated to obtain 1-chloro-(un)substituted-2-nitrobenzene 3 with fuming nitric acid, preferably without the temperature exceeding about 25° C. Ethyl 2-cyano-2-(un)substituted-2-nitrophenyl)acetate 4 can be prepared by reacting compound 3 with ethylcyanoacetate in the presence of potassium-tert-butoxide in THF (yielded compound 4 at 31.3%). Reduction of ester 4, can be carried out with excess of Zn dust (4-6 eq) using known conditions to give an ethyl 2-amino-5,6-dimethoxy-1H-indole-3-carboxylate 5 without an N-hydroxy side product.

Cyclization of ethyl 2-amino-5,6-dimethoxy-1H-indole-3-carboxylate 5 to the corresponding dihydro-4H-pyrimido[4,5-b]indoles 6, can be performed by heating at about 200-220° C. in formamide and catalytic sodium methoxide. The dihydro-pyrimidines can be converted to 4-chlorides 7 in good yields with thionylchloride and/or POCl₃ in dioxane solvent. The 4-chlorides can be utilized in preparing 4-piperazine substituted tricyclic analogues as outlined in Scheme 4. The 4-chlorides can be reacted with piperazine in the presence of pyridine in dioxane solvent at reflux temperature to give compound 8 in good yields.

Certain intermediates that can be utilized in the preparation of target compounds are outlined in Scheme 4. The variously substituted aromatic amines can be treated with thiophosgene in dichloromethane in presence of CaCO₃ and water to give isothiocyanate analogue 29, 14 and compound 2-4 in high yields.

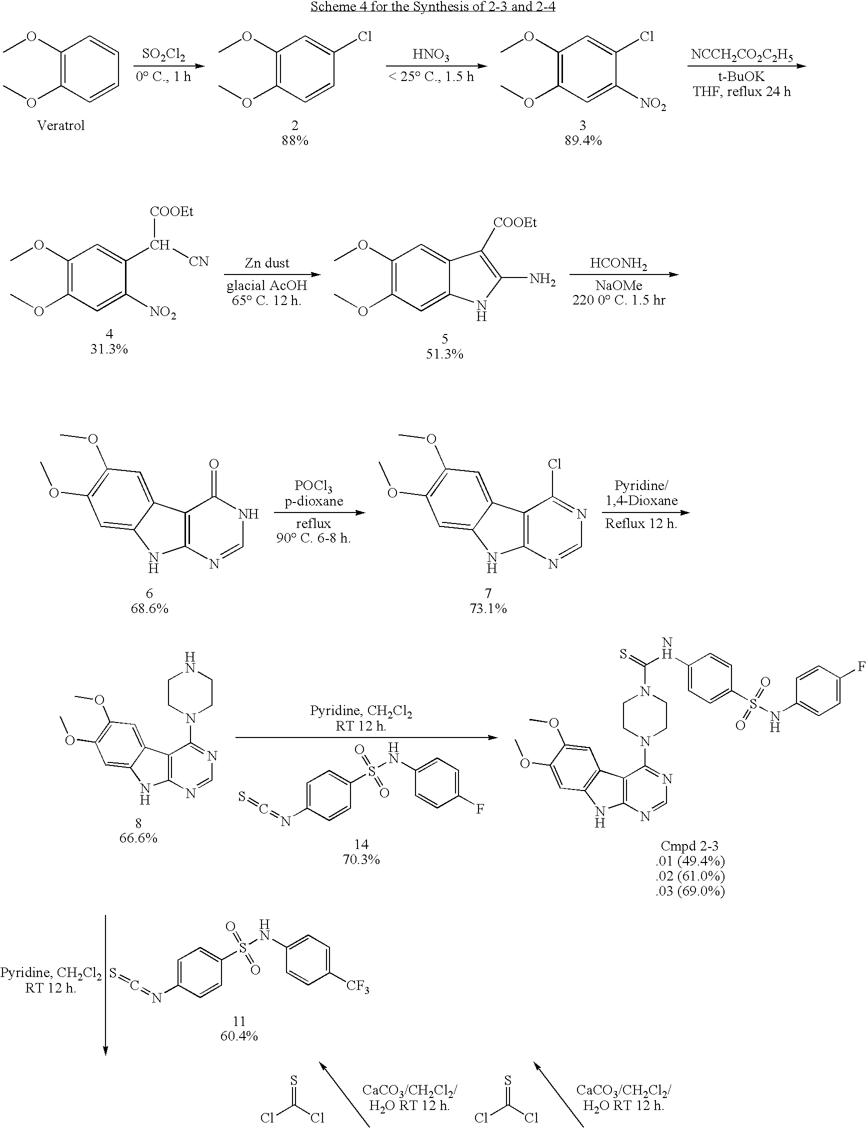

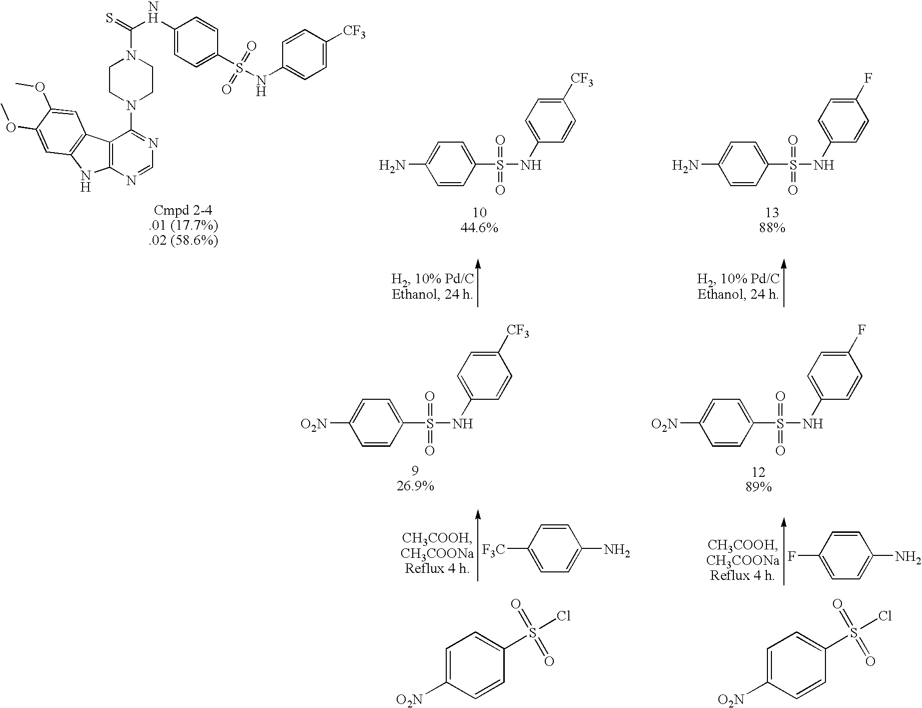

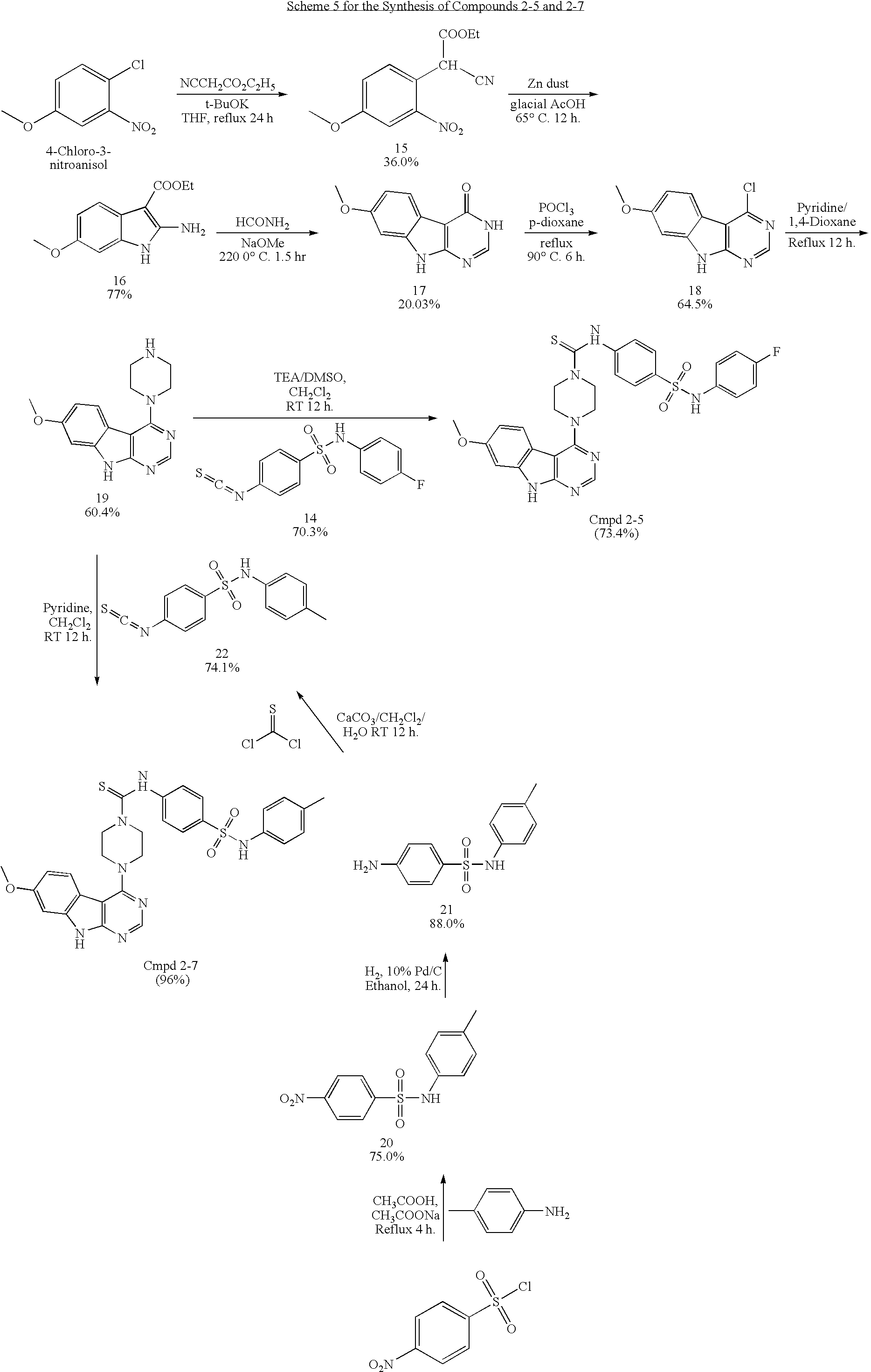

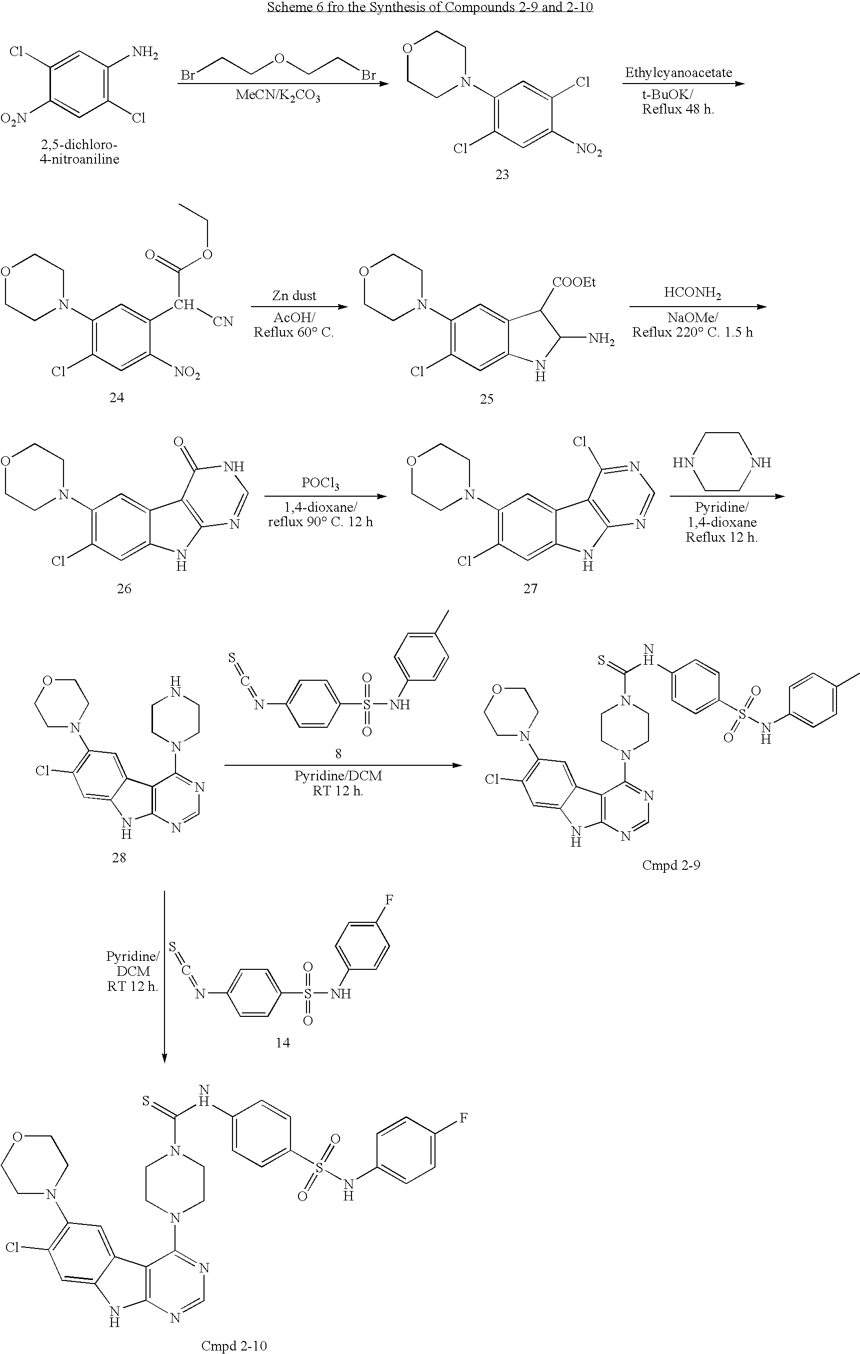

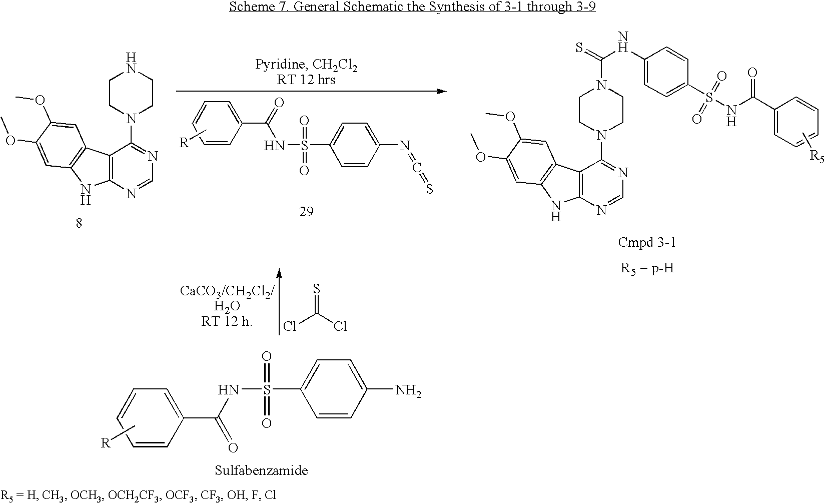

A compound of the present invention or a pharmaceutically acceptable salt thereof, can be administered as such to a human patient or can be administered in pharmaceutical compositions in which the foregoing materials are mixed with suitable carriers or excipient(s). Techniques for formulation and administration of drugs may be found, for example, in REMINGTON'S PHARMACOLOGICAL SCIENCES, Mack Publishing Co., Easton, Pa., latest edition.

A “pharmaceutical composition” refers to a mixture of one or more of the compounds described herein, or pharmaceutically acceptable salts or prodrugs thereof, with other chemical components, such as pharmaceutically acceptable excipients. The purpose of a pharmaceutical composition is to facilitate administration of a compound to an organism.

“Pharmaceutically acceptable excipient” refers to an inert substance added to a pharmaceutical composition to further facilitate administration of a compound. Examples, without limitation, of excipients include calcium carbonate, calcium phosphate, various sugars and types of starch, cellulose derivatives, gelatin, vegetable oils and polyethylene glycols.

“Pharmaceutically acceptable salt” refers to those salts which retain the biological effectiveness and properties of the parent compound. Such salts may include: (1) acid addition salt which is obtained by reaction of the free base of the parent compound with inorganic acids such as hydrochloric acid, hydrobromic acid, nitric acid, phosphoric acid, sulfuric acid, and perchloric acid and the like, or with organic acids such as acetic acid, oxalic acid, (D)- or (L)-malic acid, maleic acid, methanesulfonic acid, ethanesulfonic acid, p-toluenesulfonic acid, salicylic acid, tartaric acid, citric acid, succinic acid or malonic acid and the like, preferably hydrochloric acid or (L)-malic acid; or (2) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, tromethamine, N-methylglucamine, and the like.

The compound of the present invention may also act, or be designed to act, as a prodrug. A “prodrug” refers to an agent, which is converted into the parent drug in vivo. Prodrugs are often useful because, in some situations, they may be easier to administer than the parent drug. They may, for instance, be bioavailable by oral administration whereas the parent drug is not. The prodrug may also have improved solubility in pharmaceutical compositions over the parent drug. An example, without limitation, of a prodrug would be a compound of the present invention, which is, administered as an ester (the “prodrug”), phosphate, amide, carbamate or urea.

“Therapeutically effective amount” refers to that amount of the compound being administered which will relieve to some extent one or more of the symptoms of the disorder being treated. In reference to the treatment of cancer, a therapeutically effective amount refers to that amount which has the effect of: (1) reducing the size of the tumor; (2) inhibiting tumor metastasis; (3) inhibiting tumor growth; and/or (4) relieving one or more symptoms associated with the cancer.

The term “protein kinase-mediated condition” or “disease”, as used herein, means any disease or other deleterious condition in which a protein kinase is known to play a role. The term “protein kinase-mediated condition” or “disease” also means those diseases or conditions that are alleviated by treatment with a protein kinase inhibitor. Such conditions include, without limitation, cancer and other hyperproliferative disorders. In certain embodiments, the cancer is a cancer of colon, breast, stomach, prostate, pancreas, or ovarian tissue.

The term “Aurora-2 kinase-mediated condition” or “disease”, as used herein, means any disease or other deleterious condition in which Aurora is known to play a role. The term “Aurora-2 kinase-mediated condition” or “disease” also means those diseases or conditions that are alleviated by treatment with an Aurora-2 inhibitor.

The term “Axl kinase-mediated condition” or “disease”, as used herein, means any disease or other deleterious condition in which Axl kinase is known to play a role. The term “Axl kinase-mediated condition” or “disease” also means those diseases or conditions that are alleviated by treatment with an Axl kinase inhibitor.

As used herein, “administer” or “administration” refers to the delivery of an inventive compound or of a pharmaceutically acceptable salt thereof or of a pharmaceutical composition containing an inventive compound or a pharmaceutically acceptable salt thereof of this invention to an organism for the purpose of prevention or treatment of a protein kinase-related disorder.

Suitable routes of administration may include, without limitation, oral, rectal, transmucosal or intestinal administration or intramuscular, subcutaneous, intramedullary, intrathecal, direct intraventricular, intravenous, intravitreal, intraperitoneal, intranasal, or intraocular injections. In certain embodiments, the preferred routes of administration are oral and intravenous.

Alternatively, one may administer the compound in a local rather than systemic manner, for example, via injection of the compound directly into a solid tumor, often in a depot or sustained release formulation.

Furthermore, one may administer the drug in a targeted drug delivery system, for example, in a liposome coated with tumor-specific antibody. In this way, the liposomes may be targeted to and taken up selectively by the tumor.

Pharmaceutical compositions of the present invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with the present invention may be formulated in any conventional manner using one or more physiologically acceptable carriers comprising excipients and auxiliaries which facilitate processing of the active compounds into preparations which can be used pharmaceutically. Proper formulation is dependent upon the route of administration chosen.

For injection, the compounds of the invention may be formulated in aqueous solutions, preferably in physiologically compatible buffers such as Hanks' solution, Ringer's solution, or physiological saline buffer. For transmucosal administration, penetrants appropriate to the barrier to be permeated are used in the formulation. Such penetrants are generally known in the art.

For oral administration, the compounds can be formulated by combining the active compounds with pharmaceutically acceptable carriers well known in the art. Such carriers enable the compounds of the invention to be formulated as tablets, pills, lozenges, dragees, capsules, liquids, gels, syrups, slurries, suspensions and the like, for oral ingestion by a patient. Pharmaceutical preparations for oral use can be made using a solid excipient, optionally grinding the resulting mixture, and processing the mixture of granules, after adding other suitable auxiliaries if desired, to obtain tablets or dragee cores. Useful excipients are, in particular, fillers such as sugars, including lactose, sucrose, mannitol, or sorbitol, cellulose preparations such as, for example, maize starch, wheat starch, rice starch and potato starch and other materials such as gelatin, gum tragacanth, methyl cellulose, hydroxypropylmethyl-cellulose, sodium carboxymethylcellulose, and/or polyvinyl-pyrrolidone (PVP). If desired, disintegrating agents may be added, such as cross-linked polyvinyl pyrrolidone, agar, or alginic acid. A salt such as sodium alginate may also be used.

Dragee cores are provided with suitable coatings. For this purpose, concentrated sugar solutions may be used which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical compositions which can be used orally include push-fit capsules made of gelatin, as well as soft, sealed capsules made of gelatin and a plasticizer, such as glycerol or sorbitol. The push-fit capsules can contain the active ingredients in admixture with a filler such as lactose, a binder such as starch, and/or a lubricant such as talc or magnesium stearate and, optionally, stabilizers. In soft capsules, the active compounds may be dissolved or suspended in suitable liquids, such as fatty oils, liquid paraffin, or liquid polyethylene glycols. Stabilizers may be added in these formulations, also. Pharmaceutical compositions which may also be used include hard gelatin capsules. The capsules or pills may be packaged into brown glass or plastic bottles to protect the active compound from light. The containers containing the active compound capsule formulation are preferably stored at controlled room temperature (15-30° C.).

For administration by inhalation, the compounds for use according to the present invention may be conveniently delivered in the form of an aerosol spray using a pressurized pack or a nebulizer and a suitable propellant, e.g., without limitation, dichlorodifluoromethane, trichlorofluoromethane, dichlorotetra-fluoroethane or carbon dioxide. In the case of a pressurized aerosol, the dosage unit may be controlled by providing a valve to deliver a metered amount. Capsules and cartridges of, for example, gelatin for use in an inhaler or insufflator may be formulated containing a powder mix of the compound and a suitable powder base such as lactose or starch.

The compounds may also be formulated for parenteral administration, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulating materials such as suspending, stabilizing and/or dispersing agents.

Pharmaceutical compositions for parenteral administration include aqueous solutions of a water soluble form, such as, without limitation, a salt, of the active compound. Additionally, suspensions of the active compounds may be prepared in a lipophilic vehicle. Suitable lipophilic vehicles include fatty oils such as sesame oil, synthetic fatty acid esters such as ethyl oleate and triglycerides, or materials such as liposomes. Aqueous injection suspensions may contain substances which increase the viscosity of the suspension, such as sodium carboxymethyl cellulose, sorbitol, or dextran. Optionally, the suspension may also contain suitable stabilizers and/or agents that increase the solubility of the compounds to allow for the preparation of highly concentrated solutions.

Alternatively, the active ingredient may be in powder form for constitution with a suitable vehicle, e.g., sterile, pyrogen-free water, before use.

The compounds may also be formulated in rectal compositions such as suppositories or retention enemas, using, e.g., conventional suppository bases such as cocoa butter or other glycerides.

In addition to the formulations described previously, the compounds may also be formulated as depot preparations. Such long acting formulations may be administered by implantation (for example, subcutaneously or intramuscularly) or by intramuscular injection. A compound of this invention may be formulated for this route of administration with suitable polymeric or hydrophobic materials (for instance, in an emulsion with a pharmacologically acceptable oil), with ion exchange resins, or as a sparingly soluble derivative such as, without limitation, a sparingly soluble salt.

A non-limiting example of a pharmaceutical carrier for the hydrophobic compounds of the invention is a cosolvent system comprising benzyl alcohol, a nonpolar surfactant, a water-miscible organic polymer and an aqueous phase such as the VPD cosolvent system. VPD is a solution of 3% w/v benzyl alcohol, 8% w/v of the nonpolar surfactant polysorbate 80, and 65% w/v polyethylene glycol 300, made up to volume in absolute ethanol. The VPD cosolvent system (VPD:D5W) consists of VPD diluted 1:1 with a 5% dextrose in water solution. This cosolvent system dissolves hydrophobic compounds well, and itself produces low toxicity upon systemic administration. Naturally, the proportions of such a cosolvent system may be varied considerably without destroying its solubility and toxicity characteristics. Furthermore, the identity of the cosolvent components may be varied: for example, other low-toxicity nonpolar surfactants may be used instead of polysorbate 80, the fraction size of polyethylene glycol may be varied, other biocompatible polymers may replace polyethylene glycol, e.g., polyvinyl pyrrolidone, and other sugars or polysaccharides may substitute for dextrose.

Alternatively, other delivery systems for hydrophobic pharmaceutical compounds may be employed. Liposomes and emulsions are well known examples of delivery vehicles or carriers for hydrophobic drugs. In addition, certain organic solvents such as dimethylsulfoxide also may be employed, although often at the cost of greater toxicity.

Additionally, the compounds may be delivered using a sustained-release system, such as semipermeable matrices of solid hydrophobic polymers containing the therapeutic agent. Various sustained-release materials have been established and are well known by those skilled in the art. Sustained-release capsules may, depending on their chemical nature, release the compounds for a few weeks up to over 100 days. Depending on the chemical nature and the biological stability of the therapeutic reagent, additional strategies for protein stabilization may be employed.

The pharmaceutical compositions herein also may comprise suitable solid or gel phase carriers or excipients. Examples of such carriers or excipients include, but are not limited to, calcium carbonate, calcium phosphate, various sugars, starches, cellulose derivatives, gelatin, and polymers such as polyethylene glycols.

Many of the protein kinase-modulating compounds of the invention may be provided as physiologically acceptable salts wherein the claimed compound may form the negatively or the positively charged species. Examples of salts in which the compound forms the positively charged moiety include, without limitation, quaternary ammonium (defined elsewhere herein), salts such as the hydrochloride, sulfate, carbonate, lactate, tartrate, malate, maleate, succinate wherein the nitrogen atom of the quaternary ammonium group is a nitrogen of the selected compound of this invention which has reacted with the appropriate acid. Salts in which a compound of this invention forms the negatively charged species include, without limitation, the sodium, potassium, calcium and magnesium salts formed by the reaction of a carboxylic acid group in the compound with an appropriate base (e.g. sodium hydroxide (NaOH), potassium hydroxide (KOH), calcium hydroxide (Ca(OH)₂), etc.).

Pharmaceutical compositions suitable for use in the present invention include compositions wherein the active ingredients are contained in an amount sufficient to achieve the intended purpose, e.g., the modulation of protein kinase activity and/or the treatment or prevention of a protein kinase-related disorder.

More specifically, a therapeutically effective amount means an amount of compound effective to prevent, alleviate or ameliorate symptoms of disease or prolong the survival of the subject being treated.

Determination of a therapeutically effective amount is well within the capability of those skilled in the art, especially in light of the detailed disclosure provided herein.

For any compound used in the methods of the invention, the therapeutically effective amount or dose can be estimated initially from cell culture assays. Then, the dosage can be formulated for use in animal models so as to achieve a circulating concentration range that includes the IC₅₀ as determined in cell culture (i.e., the concentration of the test compound which achieves a half-maximal inhibition of the protein kinase activity). Such information can then be used to more accurately determine useful doses in humans.

Toxicity and therapeutic efficacy of the compounds described herein can be determined by standard pharmaceutical procedures in cell cultures or experimental animals, e.g., by determining the IC₅₀ and the LD₅₀ (both of which are discussed elsewhere herein) for a subject compound. The data obtained from these cell culture assays and animal studies can be used in formulating a range of dosage for use in humans. The dosage may vary depending upon the dosage form employed and the route of administration utilized. The exact formulation, route of administration and dosage can be chosen by the individual physician in view of the patient's condition. (See, e.g., GOODMAN & GILMAN'S THE PHARMACOLOGICAL BASIS OF THERAPEUTICS_{, Ch.} 3, 9^{th} ed., Ed. by Hardman, J., and Limbard, L., McGraw-Hill, New York City, 1996, p. 46.)

Dosage amount and interval may be adjusted individually to provide plasma levels of the active species which are sufficient to maintain the kinase modulating effects. These plasma levels are referred to as minimal effective concentrations (MECs). The MEC will vary for each compound but can be estimated from in vitro data, e.g., the concentration necessary to achieve 50-90% inhibition of a kinase may be ascertained using the assays described herein. Dosages necessary to achieve the MEC will depend on individual characteristics and route of administration. HPLC assays or bioassays can be used to determine plasma concentrations.

Dosage intervals can also be determined using MEC value. Compounds should be administered using a regimen that maintains plasma levels above the MEC for 10-90% of the time, preferably between 30-90% and most preferably between 50-90%.

At present, the therapeutically effective amounts of compounds of the present invention may range from approximately 2.5 mg/m² to 1500 mg/m² per day. Additional illustrative amounts range from 0.2-1000 mg/qid, 2-500 mg/qid, and 20-250 mg/qid.

In cases of local administration or selective uptake, the effective local concentration of the drug may not be related to plasma concentration, and other procedures known in the art may be employed to determine the correct dosage amount and interval.

The amount of a composition administered will, of course, be dependent on the subject being treated, the severity of the affliction, the manner of administration, the judgment of the prescribing physician, etc.

The compositions may, if desired, be presented in a pack or dispenser device, such as an FDA approved kit, which may contain one or more unit dosage forms containing the active ingredient. The pack may for example comprise metal or plastic foil, such as a blister pack. The pack or dispenser device may be accompanied by instructions for administration. The pack or dispenser may also be accompanied by a notice associated with the container in a form prescribed by a governmental agency regulating the manufacture, use or sale of pharmaceuticals, which notice is reflective of approval by the agency of the form of the compositions or of human or veterinary administration. Such notice, for example, may be of the labeling approved by the U.S. Food and Drug Administration for prescription drugs or of an approved product insert. Compositions comprising a compound of the invention formulated in a compatible pharmaceutical carrier may also be prepared, placed in an appropriate container, and labeled for treatment of an indicated condition. Suitable conditions indicated on the label may include treatment of a tumor, inhibition of angiogenesis, treatment of fibrosis, diabetes, and the like.

As mentioned above, the compounds and compositions of the invention will find utility in a broad range of diseases and conditions mediated by protein kinases, including diseases and conditions mediated by aurora-2 kinase. Such diseases may include by way of example and not limitation, cancers such as lung cancer, NSCLC (non small cell lung cancer), oat-cell cancer, bone cancer, pancreatic cancer, skin cancer, dermatofibrosarcoma protuberans, cancer of the head and neck, cutaneous or intraocular melanoma, uterine cancer, ovarian cancer, colo-rectal cancer, cancer of the anal region, stomach cancer, colon cancer, breast cancer, gynecologic tumors (e.g., uterine sarcomas, carcinoma of the fallopian tubes, carcinoma of the endometrium, carcinoma of the cervix, carcinoma of the vagina or carcinoma of the vulva), Hodgkin's Disease, hepatocellular cancer, cancer of the esophagus, cancer of the small intestine, cancer of the endocrine system (e.g., cancer of the thyroid, pancreas, parathyroid or adrenal glands), sarcomas of soft tissues, cancer of the urethra, cancer of the penis, prostate cancer (particularly hormone-refractory), chronic or acute leukemia, solid tumors of childhood, hypereosinophilia, lymphocytic lymphomas, cancer of the bladder, cancer of the kidney or ureter (e.g., renal cell carcinoma, carcinoma of the renal pelvis), pediatric malignancy, neoplasms of the central nervous system (e.g., primary CNS lymphoma, spinal axis tumors, medulloblastoma, brain stem gliomas or pituitary adenomas), Barrett's esophagus (pre-malignant syndrome), neoplastic cutaneous disease, psoriasis, mycoses fungoides, and benign prostatic hypertrophy, diabetes related diseases such as diabetic retinopathy, retinal ischemia, and retinal neovascularization, hepatic cirrhosis, angiogenesis, cardiovascular disease such as atherosclerosis, immunological disease such as autoimmune disease and renal disease.

The inventive compound can be used in combination with one or more other chemotherapeutic agents. The dosage of the inventive compounds may be adjusted for any drug-drug reaction. In one embodiment, the chemotherapeutic agent is selected from the group consisting of mitotic inhibitors, alkylating agents, anti-metabolites, cell cycle inhibitors, enzymes, topoisomerase inhibitors such as CAMPTOSAR (irinotecan), biological response modifiers, anti-hormones, antiangiogenic agents such as MMP-2, MMP-9 and COX-2 inhibitors, anti-androgens, platinum coordination complexes (cisplatin, etc.), substituted ureas such as hydroxyurea; methylhydrazine derivatives, e.g., procarbazine; adrenocortical suppressants, e.g., mitotane, aminoglutethimide, hormone and hormone antagonists such as the adrenocorticosteriods (e.g., prednisone), progestins (e.g., hydroxyprogesterone caproate), estrogens (e.g., diethylstilbesterol), antiestrogens such as tamoxifen, androgens, e.g., testosterone propionate, and aromatase inhibitors, such as anastrozole, and AROMASIN (exemestane).

Examples of alkylating agents that the above method can be carried out in combination with include, without limitation, fluorouracil (5-FU) alone or in further combination with leukovorin; other pyrimidine analogs such as UFT, capecitabine, gemcitabine and cytarabine, the alkyl sulfonates, e.g., busulfan (used in the treatment of chronic granulocytic leukemia), improsulfan and piposulfan; aziridines, e.g., benzodepa, carboquone, meturedepa and uredepa; ethyleneimines and methylmelamines, e.g., altretamine, triethylenemelamine, triethylenephosphoramide, triethylenethiophosphoramide and trimethylolmelamine; and the nitrogen mustards, e.g., chlorambucil (used in the treatment of chronic lymphocytic leukemia, primary macroglobulinemia and non-Hodgkin's lymphoma), cyclophosphamide (used in the treatment of Hodgkin's disease, multiple myeloma, neuroblastoma, breast cancer, ovarian cancer, lung cancer, Wilm's tumor and rhabdomyosarcoma), estramustine, ifosfamide, novembrichin, prednimustine and uracil mustard (used in the treatment of primary thrombocytosis, non-Hodgkin's lymphoma, Hodgkin's disease and ovarian cancer); and triazines, e.g., dacarbazine (used in the treatment of soft tissue sarcoma).

Examples of antimetabolite chemotherapeutic agents that the above method can be carried out in combination with include, without limitation, folic acid analogs, e.g., methotrexate (used in the treatment of acute lymphocytic leukemia, choriocarcinoma, mycosis fungiodes, breast cancer, head and neck cancer and osteogenic sarcoma) and pteropterin; and the purine analogs such as mercaptopurine and thioguanine which find use in the treatment of acute granulocytic, acute lymphocytic and chronic granulocytic leukemias.

Examples of natural product-based chemotherapeutic agents that the above method can be carried out in combination with include, without limitation, the vinca alkaloids, e.g., vinblastine (used in the treatment of breast and testicular cancer), vincristine and vindesine; the epipodophyllotoxins, e.g., etoposide and teniposide, both of which are useful in the treatment of testicular cancer and Kaposi's sarcoma; the antibiotic chemotherapeutic agents, e.g., daunorubicin, doxorubicin, epirubicin, mitomycin (used to treat stomach, cervix, colon, breast, bladder and pancreatic cancer), dactinomycin, temozolomide, plicamycin, bleomycin (used in the treatment of skin, esophagus and genitourinary tract cancer); and the enzymatic chemotherapeutic agents such as L-asparaginase.

Examples of useful COX-II inhibitors include Vioxx, CELEBREX (celecoxib), valdecoxib, paracoxib, rofecoxib, and Cox 189.

Examples of useful matrix metalloproteinase inhibitors are described in WO 96/33172 (published Oct. 24, 1996), WO 96/27583 (published Mar. 7, 1996), European Patent Application No. 97304971.1 (filed Jul. 8, 1997), European Patent Application No. 99308617.2 (filed Oct. 29, 1999), WO 98/07697 (published Feb. 26, 1998), WO 98/03516 (published Jan. 29, 1998), WO 98/34918 (published Aug. 13, 1998), WO 98/34915 (published Aug. 13, 1998), WO 98/33768 (published Aug. 6, 1998), WO 98/30566 (published Jul. 16, 1998), European Patent Publication 606,046 (published Jul. 13, 1994), European Patent Publication 931,788 (published Jul. 28, 1999), WO 90/05719 (published May 31, 1990), WO 99/52910 (published Oct. 21, 1999), WO 99/52889 (published Oct. 21, 1999), WO 99/29667 (published Jun. 17, 1999), PCT International Application No. PCT/IB98/01113 (filed Jul. 21, 1998), European Patent Application No. 99302232.1 (filed Mar. 25, 1999), Great Britain patent application number 9912961.1 (filed Jun. 3, 1999), U.S. Pat. No. 5,863,949 (issued Jan. 26, 1999), U.S. Pat. No. 5,861,510 (issued Jan. 19, 1999), and European Patent Publication 780,386 (published Jun. 25, 1997), all of which are incorporated herein in their entireties by reference. Preferred MMP-2 and MMP-9 inhibitors are those that have little or no activity inhibiting MMP-1. More preferred are those that selectively inhibit MMP-2 and/or MMP-9 relative to the other matrix-metalloproteinases (i.e., MMP-1, MMP-3, MMP-4, MMP-5, MMP-6, MMP-7, MMP-8, MMP-10, MMP-11, MMP-12, and MMP-13).

Some specific examples of MMP inhibitors useful in the present invention are AG-3340, RO 32-3555, RS 13-0830, and compounds selected from: 3-[[4-(4-fluoro-phenoxy)-benzenesulfonyl]-(1-hydroxycarbamoyl-cyclopentyl)-amino]-propionic acid; 3-exo-3-[4-(4-fluoro-phenoxy)-benzenesulfonylamino]-8-oxa-bicyclo[3.2.1]octane-3-carboxylic acid hydroxyamide; (2R,3R) 1-[4-(2-chloro-4-fluoro-benzyloxy)-benzenesulfonyl]-3-hydroxy-3-methyl-piperidine-2-carboxylic acid hydroxyamide; 4-[4-(4-fluoro-phenoxy)-benzenesulfonylamino]-tetrahydro-pyran-4-carboxylic acid hydroxyamide; 3-[[4-(4-fluoro-phenoxy)-benzenesulfonyl]-(1-hydroxycarbamoyl-cyclobutyl)-amino]-propionic acid; 4-[4-(4-chloro-phenoxy)-benzenesulfonylamino]-tetrahydro-pyran-4-carboxylic acid hydroxyamide; (R) 3-[4-(4-chloro-phenoxy)-benzenesulfonylamino]-tetrahydro-pyran-3-carboxylic acid hydroxyamide; (2R,3R) 1-[4-(4-fluoro-2-methylbenzyloxy)-benzenesulfonyl]-3-hydroxy-3-methyl-piperidine-2-carboxylic acid hydroxyamide; 3-[[(4-(4-fluoro-phenoxy)-benzenesulfonyl]-(1-hydroxycarbamoyl-1-methyl-ethyl)-amino]-propionic acid; 3-[[4-(4-fluoro-phenoxy)-benzenesulfonyl]-(4-hydroxycarbamoyl-tetrahydro-pyran-4-yl)-amino]-propionic acid; 3-exo-3-[4-(4-chloro-phenoxy)-benzenesulfonylamino]-8-oxa-bicyclo[3.2.1]octane-3-carboxylic acid hydroxyamide; 3-endo-3-[4-(4-fluoro-phenoxy)-benzenesulfonylamino]-8-oxa-bicyclo[3.2.1]octane-3-carboxylic acid hydroxyamide; and (R) 3-[4-(4-fluoro-phenoxy)-benzenesulfonylamino]-tetrahydro-furan-3-carboxylic acid hydroxyamide; and pharmaceutically acceptable salts and solvates of these compounds.

Other anti-angiogenesis agents, other COX-II inhibitors and other MMP inhibitors, can also be used in the present invention.

An inventive compound can also be used with other signal transduction inhibitors, such as agents that can inhibit EGFR (epidermal growth factor receptor) responses, such as EGFR antibodies, EGF antibodies, and molecules that are EGFR inhibitors; VEGF (vascular endothelial growth factor) inhibitors; and erbB2 receptor inhibitors, such as organic molecules or antibodies that bind to the erbB2 receptor, such as HERCEPTIN (Genentech, Inc., South San Francisco, Calif.). EGFR inhibitors are described in, for example in WO 95/19970 (published Jul. 27, 1995), WO 98/14451 (published Apr. 9, 1998), WO 98/02434 (published Jan. 22, 1998), and U.S. Pat. No. 5,747,498 (issued May 5, 1998), and such substances can be used in the present invention as described herein.

EGFR-inhibiting agents include, but are not limited to, the monoclonal antibodies C225 and anti-EGFR 22Mab (ImClone Systems, Inc., New York, N.Y.), the compounds ZD-1839 (AstraZeneca), BIBX-1382 (Boehringer Ingelheim), MDX-447 (Medarex Inc., Annandale, N.J.), and OLX-103 (Merck & Co., Whitehouse Station, N.J.), and EGF fusion toxin (Seragen Inc., Hopkinton, Mass.).

These and other EGFR-inhibiting agents can be used in the present invention. VEGF inhibitors, for example SU-5416 and SU-6668 (Sugen Inc., South San Francisco, Calif.), can also be combined with an inventive compound. VEGF inhibitors are described in, for example, WO 01/60814 A3 (published Aug. 23, 2001), WO 99/24440 (published May 20, 1999), PCT International Application PCT/IB99/00797 (filed May 3, 1999), WO 95/21613 (published Aug. 17, 1995), WO 99/61422 (published Dec. 2, 1999), U.S. Pat. No. 5,834,504 (issued Nov. 10, 1998), WO 01/60814, WO 98/50356 (published Nov. 12, 1998), U.S. Pat. No. 5,883,113 (issued Mar. 16, 1999), U.S. Pat. No. 5,886,020 (issued Mar. 23, 1999), U.S. Pat. No. 5,792,783 (issued Aug. 11, 1998), WO 99/10349 (published Mar. 4, 1999), WO 97/32856 (published Sep. 12, 1997), WO 97/22596 (published Jun. 26, 1997), WO 98/54093 (published Dec. 3, 1998), WO 98/02438 (published Jan. 22, 1998), WO 99/16755 (published Apr. 8, 1999), and WO 98/02437 (published Jan. 22, 1998), all of which are incorporated herein in their entireties by reference. Other examples of some specific VEGF inhibitors useful in the present invention are IM862 (Cytran Inc., Kirkland, Wash.); anti-VEGF monoclonal antibody of Genentech, Inc.; and angiozyme, a synthetic ribozyme from Ribozyme (Boulder, Colo.) and Chiron (Emeryville, Calif.). These and other VEGF inhibitors can be used in the present invention as described herein. pErbB2 receptor inhibitors, such as GW-282974 (Glaxo Wellcome plc), and the monoclonal antibodies AR-209 (Aronex Pharmaceuticals Inc., The Woodlands, Tex.) and 2B-1 (Chiron), can furthermore be combined with an inventive compound, for example, those indicated in WO 98/02434 (published Jan. 22, 1998), WO 99/35146 (published Jul. 15, 1999), WO 99/35132 (published Jul. 15, 1999), WO 98/02437 (published Jan. 22, 1998), WO 97/13760 (published Apr. 17, 1997), WO 95/19970 (published Jul. 27, 1995), U.S. Pat. No. 5,587,458 (issued Dec. 24, 1996), and U.S. Pat. No. 5,877,305 (issued Mar. 2, 1999), which are all hereby incorporated herein in their entireties by reference. ErbB2 receptor inhibitors useful in the present invention are also described in U.S. Pat. No. 6,284,764 (issued Sep. 4, 2001), incorporated in its entirety herein by reference. The erbB2 receptor inhibitor compounds and substance described in the aforementioned PCT applications, U.S. patents, and U.S. provisional applications, as well as other compounds and substances that inhibit the erbB2 receptor, can be used with an inventive compound, in accordance with the present invention.

An inventive compound can also be used with other agents useful in treating cancer, including, but not limited to, agents capable of enhancing antitumor immune responses, such as CTLA4 (cytotoxic lymphocyte antigen 4) antibodies, and other agents capable of blocking CTLA4; and anti-proliferative agents such as other farnesyl protein transferase inhibitors, for example the farnesyl protein transferase inhibitors described in the references cited in the “Background” section, of U.S. Pat. No., 6,258,824 B1.

The above method can also be carried out in combination with radiation therapy, wherein the amount of an inventive compound in combination with the radiation therapy is effective in treating the above diseases.

Techniques for administering radiation therapy are known in the art, and these techniques can be used in the combination therapy described herein. The administration of the compound of the invention in this combination therapy can be determined as described herein.

The invention will be further understood upon consideration of the following non-limiting Examples.

### EXAMPLES

### Example 1

### Chemical Synthesis of Kinase Inhibitors

¹H NMR spectra were recorded on a Varian 400 spectrometer, using the solvent as internal standard. Chemical shifts are expressed in ppm (δ). Proton magnetic resonance chemical shift values were measure in deuterated CDCl3 or DMSO-d6 unless otherwise stated. ESI mass spectra (MS) were obtained on a VG-Quattro II and PE-SEIEX (API) mass spectrometer. Thin-layer chromatography was performed on Merck Kieselgel silica 60 plates coated with 250 μm layer with fluorescent indicator. Components were visualized by UV light (λ=254 nm) and or by iodine vapor. Flash column chromatographic separations were carried out on 70-230 mesh 60 Å silica gel and on CombiFlash companion (Teledyne ISCO) using RediSep flash columns. All the solvents used were best grade anhydrous obtained from Aldrich. Analytical HPLC was performed on a Waters Breeze system using the following and quoted as retention time (RT) in minutes. The column used was symmetry C18 5 μm, 4.6×150 mm column (WAT045905). All experiments dealing with moisture-sensitive compounds were conducted under dry nitrogen or argon. Starting materials, unless otherwise specified, were commercially available (Aldrich, Fluka, Lancaster and TCI) and of the best grade and were used without further purification. Organic solutions, where applicable, were dried over anhydrous Na₂SO₄ and evaporated using a Yamamoto RE500 rotary evaporator at 15-20 mmHg.

### Example 2

### Preparation of 4-chloro-1,2-dimethoxy-benzene 2 in Scheme 1

In a 500 mL of three-necked flask with a thermometer, CaCl₂ guard tube and dropping funnel were introduced at 0° C. 25 g (23.06 mL, 1 eq) of veratrol 1 followed by drop by drop addition of 24.42 g (14.53 mL, 1 eq) of sulfuryl chloride. When the addition was completed, the reaction mixture was brought to RT after 1 hour, it was distilled under reduced pressure (125-130 0° C.) and the obtained yellow oil is collected and dried to give compound 2 (27.8 g, 89.6%) as yellow color liquid.

### Example 3

### Preparation of 1-chloro-4,5-dimethoxy-2-nitrobenzene 3

In a 500 mL of three-necked flask with a thermometer and dropping funnel, were charge 27.8 g (1 eq) of 1,4-chloro-1,2-dimethoxybenzene 2 followed drop by drop addition of 30.43 g (3 eq, 20.4 ml) of fuming nitric acid without the temperature being exceed to 25° C. When the addition was completed the reaction mixture was allowed to stand 1.5 h and obtained solid compound 3 was treated with water and the yellow solid was filtered and washed with water and dried (31.3 g, 89.4%) to give yellow solid.

### Example 4

### Preparation ethyl 2-cyano-2-(4,5-dimethoxy-2-nitrophenyl)acetate 4

Potassium tert-butoxide 32.28 g (2 eq) was charged into an ice cold solution of ethyl cyanoacetate 32.54 g (30.61 mL, 2 eq)) in THF (250 mL) and was stirred for 15 min. To the white suspension, the compound 3 (1-chloro-4,5-dimethoxy-2-nitrobenzene) 31.30 g (1 eq) was added after and the reaction mixture was heated to reflux for 24 hrs. The cooled reaction mixture was poured into water and extracted in to diethyl ether and the solvent was evaporated. The obtained compound crude ethyl 2-cyano-2-(4,5-dimethoxy-2-nitrophenyl)acetate 4 was purified by flash column prior to using in next step (9.5 g, 22.6%) as thick yellow oil.

### Example 5

### Preparation of ethyl 2-amino-5,6-dimethoxy-1H-indole-3-carboxylate

A solution of ethyl 2-cyano-2-(4,5-dimethoxy-2-nitrophenyl)acetate 4 9.5 g (1 eq) in AcOH 50 mL was reacted with Zn dust 8.44 g (4 eq) by heating at 65° C. for 12 hours. The reaction mixture was cooled and filtered through filter aid and was washed well with AcOH and the filtrate was concentrated to a residue was treated with water and extracted into dichloromethane and was purified by column chromatography (4.4 g, 55%) as brown solid.

### Example 6

### Preparation of 6,7-dimethoxy-3H-pyrimido[4,5-b]indol-4(9H)-one 6

A solution of ethyl 2-amino-5,6-dimethoxy-1H-indole-3-carboxylate (5) 4.4 g (1 eq), NaOMe (900 mg), and formamide (50 ml) was heated under N₂ at 220 0° C. for 2 hrs. The solution was cooled and stored for 2.5 days and filtered. The solid separated out from the formamide was filtered and washed with water and dried to obtain compound 6 (6,7-dimethoxy-4-piperazin-1-yl-9,9a-dihydro-4-aH-pyrimido[4,5-b]indole) as dark brown solid was purified by flash column chromatography (2.8 g (70%) as dark brown solid.

### Example 7

### 4-Chloro-6,7-dimethoxy-9,9a-dihydro-4-aH-pyrimido[4,5-b]indole 7

The 4-chloro-tricyclic and quinazoline building blocks were synthesized using literature methods (Pandey, A., et al., J. Med. Chem. 2002, 45:3772-93; Matsuno, K., et al., J. Med. Chem. 2002, 45:3057-66; Matsuno, K., et al., J. Med. Chem. 2002, 45:4513-23; and Venugopalan, B., et al., J. Heterocycl. Chem. 1988, 25:1633-39). A suspension of compound 6 (2.8 g), POCl₃ (20 mL) and p-dioxane 65 mL was heated at reflux for 6 hrs. The obtained mixture was cooled and the solvents were evaporated. The crude product was purified by column chromatography using 1% MeOH/DCM to give compound 7 (2.2 g, 73.3%) as pale yellow solid.

### Example 8

### 6,7-dimethoxy-4-(piperazin-1-yl)-9H-pyrimido[4,5-b]indole 8

Compound 7 was dissolved in p-dioxane (50 mL) and piprazine (3.9 g) was added following the addition of pyridine (5 mL) under argon at RT. The reaction mixture was heated to reflux for 16 hours and it was cooled. The solvents were removed under vacuum and the obtained crude product as purified by flash column chromatograph using DCM and 10% MeOH solvent system. The compound 8 obtained after purification was half white solid (3.9 g, 66.10%).

### Example 9

### Preparation N-Acetyl-4-isothiocyanato-benzenesulfonamide 13 in Scheme 2

Un(substituted) amine and or N-Acetyl-4-amino-benzenesulfonamide was dissolved in DCM 25 mL and added to a solution of 0.934 g of CaCO3 and 0.534 mL of thiophosgene dissolved in 15 mL of water. The reaction mixture was stirred overnight. The resulting mixture was extracted in to DCM and dried to leave compound 13 (0.462 g, 38.6%) as white solid.

### Example 10

### Preparation of 4-(6-Chloro-7-trifluoromethyl-9H-pyrimido[4,5-b]indol-4-yl)-piperazine-1-carbothioic acid (4-acetylsulfamoyl-phenyl)-amide (Compound No. 1 in Table 1; Compound 1-1)

To a stirred solution of compound; 6-chloro-4-(piperazin-1-yl)-7-(trifluoromethyl)-9H-pyrimido[4,5-b]indole (prepared using similar method given in example 8) in DCM was added the compound 13 followed by the addition of pyridine. The resulting reaction mixture was stirred at RT for 12 hours. After the completion of reaction, the solvents were evaporated. The crude product was purified by column chromatography using DCM and 5% MeOH solvent system (0.108 g, 97%) as white solid.

### Example 11

### Preparation of 4-(6,7-Dimethoxy-9H-pyrimido[4,5-b]indol-4-yl)-piperazine-1-carbothioic acid (4-acetylsulfamoyl-phenyl)-amide, (Compound No. 2 in Table 1; Compound 1-2)

To a stirred solution of compound 8 (prepared as shown in example 8) in DCM was added the compound 13 followed by the addition of pyridine. The resulting reaction mixture was stirred at RT for 12 hours. After the completion of reaction, the solvents were evaporated. The crude product was purified by column chromatography using DCM and 5% MeOH solvent system (0.043 g, 59.1% as white solid.

### Example 12

### Preparation of 4-(6-chloro-9H-pyrimido[4,5-b]indol-4-yl)-piperazine-1-carbothioic acid (4-acetylsulfamoyl-phenyl)-amide (Compound 3 in Table 1; Compound 1-3)

To a stirred solution of compound 6-chloro-4-(piperazin-1-yl)-9H-pyrimido[4,5-b]indole (prepared using similar procedure given in Example 8) in DCM was added the compound 13 followed by the addition of pyridine. The resulting reaction mixture was stirred at RT for 12 hours. After the completion of reaction, the solvents were evaporated. The crude product was purified by CombiFlash Companion using DCM and 10% MeOH solvent system (0.12 g, 63.3%) as white solid.

### Example 13

### Preparation of 1-(3-chloropropoxy)-4-chloro-2-methoxybenzene 15 in Scheme 2

Compound 4-Chloro-2-methoxyphenol 14, cesium carbonate and 1-bromo-3-chloropropane in acetonitrile was heated to reflux for 1 hour. The reaction mixture was cooled and the solvent evaporated. The obtained residue was dissolved in water (20 mL) and extracted in to DCM. The DCM layer was washed with brine and dried. The solvent was evaporated and resulting solid was treated with ether and the solid was colleted to yield compound 15 (7.34 g, 99%) as pale yellow oil.

### Example 14

### Preparation of 1-(3-(4-chloro-2-methoxyphenoxy)propyl)-4-methylpiperazine 17 in Scheme 2

Compound 15 was dissolved in acetonitrile and was added N-methylpiperazine (2 eq) and the resulting reaction mixture was heated to 70° C. for 8 hours. The reaction mixture was cooled and the solvent was evaporated. The residue was treated with diethyl ether and the precipitated solid was filtered and dried to obtain yellowish-brown solid (5.9 g, 63.2%) as yellowish-brown solid.

### Example 15

### Preparation of 1-(3-(4-chloro-2-methoxy-5-nitrophenoxy)propyl)-4-methylpiperazine 18

Acetic acid was slowly added to nitric acid at 5° C. The powdered compound 17 was added to the mixture and stirred for 15 minutes. The resulting reaction mixture was warmed to RT and stirred overnight. The solvents were evaporated and viscous liquid is poured in to ice water and diluted with NaHCO₃ solution. The obtained mixture was evaporated and purified by silica column chromatography using 5% MeOH in dichloromethane (1.8 g, 52.1%) as yellow solid.

### Example 16

### Preparation of ethyl 2-cyano-2-(4-chloro-2-nitrophenyl)acetate

Similar methods as given for compounds 4, 5, 6 and 7 (Scheme 1 and 2) were employed to prepare the compound 7-(3-(4-methylpiperazin-1-yl)propoxy)-6-methoxy-4-(piperazin-1-yl)-9H-pyrimido[4,5-b]indole.

### Example 17

### Inhibition of Aurora-2 Kinase Activity by Compounds 1-1 and 1-2

Illustrative compounds 1-1 and 1-2 were evaluated in an aurora-2 kinase inhibition assay.

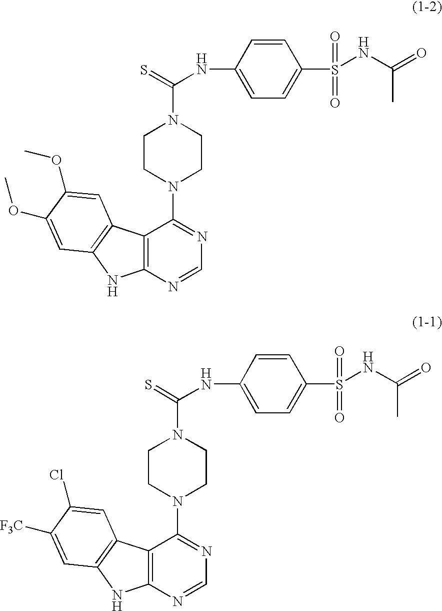

In this assay kinase activity was determined by quantifying the amount of ATP remaining in solution following a kinase reaction by measuring the light units (LU) produced by luciferase using a luminometer. Percent inhibition was determined for individual compounds by comparing luminometer readings of drug-treated reactions to controls containing no drug (DMSO control) and no Aurora-2 enzyme (ATP control) in the following equation:

$$Percent Inhibition = \frac{{LU}_{drug} - {LU}_{DMSO}}{{LU}_{ATP} - {LU}_{DMSO}} \times 100$$

In a 50 μl reaction, recombinant aurora-2 kinase produced in sf9 cells (Upstate, Lake Placid, N.Y.) was incubated at 30° C. for two hours with 62.5 μM Kemptide (Calbiochem, San Diego, Calif.), 3 μM ATP (Invitrogen, Carlsbad, Calif.) and kinase reaction buffer (40 mM Tris-HCl, 10 mM MgCl₂ and 0.1 μg/μl bovine serum albumin (BSA)). This reaction was carried out in the presence of drug substances, which had been previously diluted to desired concentrations in DMSO. After incubation, 50 μl of Kinase-Glo® (Promega, Inc., Madison, Wis.) solution was added to each reaction mixture and allowed to equilibrate for 10 minutes at room temperature. Kinase-Glo solution contains luciferase enzyme and luciferin, which react with ATP to produce light. Kinase activity is determined by quantifying the amount of ATP remaining in solution following the kinase reaction by measuring the light units (LU) produced by luciferase using a luminometer (Thermo-Electron, Vantaa, Finland).

The drug concentration at which 50% of aurora-2 kinase activity was inhibited (IC₅₀) was determined for illustrative compounds compound 1-1 and compound 1-2. The IC₅₀ for compound 1-1 was 0.049 uM, while that of compound 1-2 was <0.005 uM. This inhibitory activity for compounds 1-1 and 1-2 was unexpectedly high, particularly, for example, in comparison to significantly lower levels of activity observed for compounds structurally related to compounds 1-1 and 1-2, such as those in which the structural group:

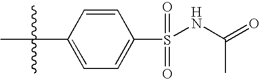

that is present on compounds 1-1 and 1-2, is replaced by one of the following:

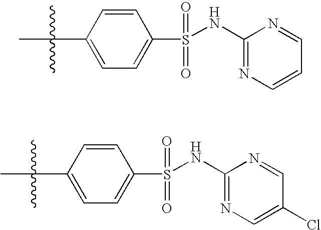

Illustrative compounds of the present invention, such as compounds 1-1 and 1-2, thus provide significantly greater inhibitory activity against aurora-2 kinase than has been observed for other structurally related compounds.

### Example 18

### Compound 1-1 Induces Cancer Cell Cytotoxicity

To evaluate cell killing of cancer cell lines, an in vitro cytotoxicity assay was performed. The tumor cell lines used were purchased from the American Type Culture Collection, and are identified as follows: Panc-1 (pancreas), MiaPaCa-2 (pancreas), MCF-7 (breast), HT-29 (colon), U2-OS (osteosarcoma), OVCAR-3 (ovary), HepG2 (hepatocellular carcinoma) and TT (medullary thyroid). The assay utilized the Cell-Titer-Glo Non-Radioactive Cell Proliferation Assay (Promega Corp., Madison, Wis.). First the cells were cultured in RPMI 1640 medium (Cat# 21870-076, Invitrogen Corporation) supplemented with 300 mg/L L-glutamine, 100 units/ml penicillin, 100 μg/ml streptomycin and 10% fetal bovine serum. All the cell lines were incubated in a humidified incubator at 37° C. with 5% CO₂ atmosphere.

Cells were plated at a density of 2000 to 10000 cells per well, depending on their growth rate, in 0.09 mL medium on day 0 in 96-well Microlite TCT microtiter plates (7418, Thermo Labsystems, Franklin, Mass.). On day 1, 10 μL of serial dilutions of the individual compounds were added to the plates in replicates of 3. After incubation for 4 days at 37° C. in a humidified incubator, the cells were lysed in the Cell-Titer-Glo reagent, which also contains luciferase enzyme. The luciferase reaction utilizes ATP released from lysed cells to produce light, the intensity of which is linearly related to the amount of ATP. Thus, the amount of light produced is a reflection of the number of cells remaining in the well after drug treatment. This luminescence was measured using a Luminoskan luminometer (Thermo Electron Corp., Vantaa, Finland) Data were expressed as the percentage of survival of control cells calculated from the luminescence corrected for background. The surviving percent of cells was determined by dividing the mean luminescence values of the treated wells by the mean luminescence values of the control and multiplying by 100.

The calculated IC₅₀ values for compound 1-1 for the following cell lines: Panc-1, MiaPaCa-2, MCF-7, HT-29, U2-OS, OVCAR-3, HepG2 and TT, were as follows: 40.67 uM, 66.59 uM, 22.46 uM, 14.65 uM, 25.93 uM, 24.97 uM, 7.83 uM and 51.67 uM, respectively. As above, the level of activity for compound 1-1 was unexpectedly high relative to the levels observed for structurally related compounds.

### Example 19

### Compound 1-1 Inhibits Tumor Growth In Vivo

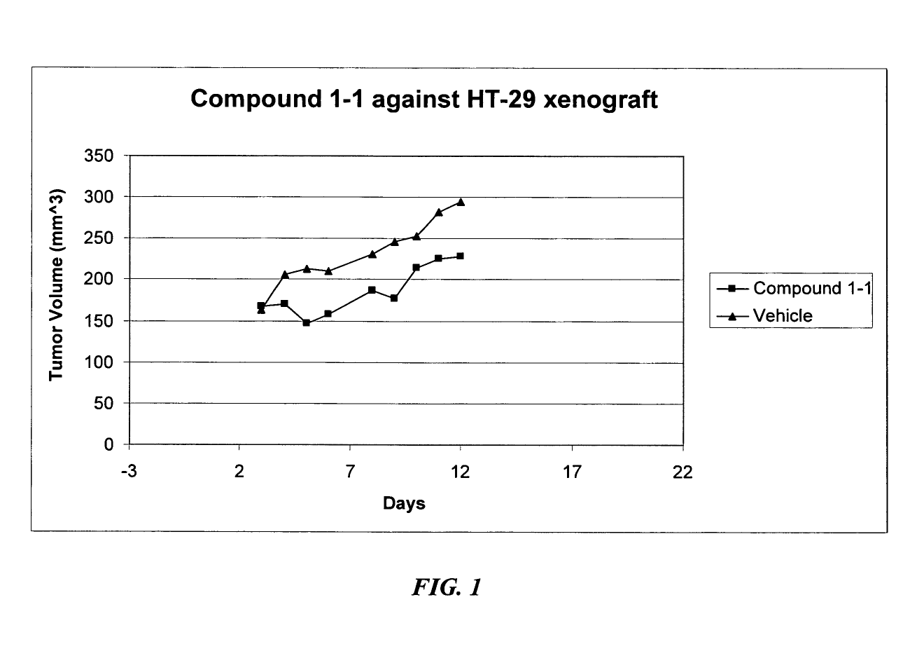

In order to evaluate the effectiveness of compound 1-1 against tumor cells in a living system, a xenograft study was performed in mice. 1×10⁷ HT-29 human colon cancer cells were injected subcutaneously into 16 Nu/Nu athymic nude mice (Charles River Laboratories, Wilmington, Mass.). Tumor volume was measured according to the formula ((Width)²*Length)/2. Tumors were allowed to grow to approximately 100 mm³ in volume (Day 0), at which point mice were randomized to two groups: Eight mice were treated with 25 mg/kg compound 1-1, while the other eight were given an equal volume of drug vehicle. For this study, the drug vehicle used was 60% propylene glycol, 30% polyethylene glycol 300, 10% ethanol with 150 mg/mL 2-hydroxypropyl-beta-cyclodextrin. Each mouse received 0.1 mL of drug or vehicle intraperitoneally on a q.d.x 5 schedule for two weeks, with two days rest between cycles. No noticeable toxicity from drug or vehicle was noted through the duration of this study. Using this approach, compound 1-1 was found to be effective for inhibiting tumor growth in vivo, the results for which are illustrated in FIG. 1.

### Example 20

### Activity of Illustrative Compounds as Determined by Aurora-2 Kinase Assays and Cancer Cell-Based Cytotoxicity Assays

Illustrative compounds described herein were evaluated in an aurora-2 kinase inhibition assay, essentially as described in Example 17 above. The compounds tested in the assay included Compounds 1-1, 1-2, 1-3, 1-4, 1-8, 1-26, 1-34, 1-42, 1-107 and 1-115, as set forth above in Table 1.

Briefly, kinase activity was determined by quantifying the amount of ATP remaining in solution following a kinase reaction by measuring the light units (LU) produced by luciferase using a luminometer. Percent inhibition was determined for individual compounds by comparing luminometer readings of drug-treated reactions to controls containing no drug (DMSO control) and no Aurora-2 enzyme (ATP control) in the following equation:

$$Percent Inhibition = \frac{{LU}_{drug} - {LU}_{DMSO}}{{LU}_{ATP} - {LU}_{DMSO}} \times 100$$

In a 50 μl reaction, recombinant aurora-2 kinase produced in sf9 cells (Upstate, Lake Placid, N.Y.) was incubated at 30° C. for two hours with 62.5 μM Kemptide (Calbiochem, San Diego, Calif.), 3 μM ATP (Invitrogen, Carlsbad, Calif.) and kinase reaction buffer (40 mM Tris-HCl, 10 mM MgCl₂ and 0.1 μg/Il bovine serum albumin (BSA)). This reaction was carried out in the presence of drug substances, which had been previously diluted to desired concentrations in DMSO. After incubation, 50 μl of Kinase-Glo® (Promega, Inc., Madison, Wis.) solution was added to each reaction mixture and allowed to equilibrate for 10 minutes at room temperature. Kinase-Glo solution contains luciferase enzyme and luciferin, which react with ATP to produce light. Kinase activity is determined by quantifying the amount of ATP remaining in solution following the kinase reaction by measuring the light units (LU) produced by luciferase using a luminometer (Thermo-Electron, Vantaa, Finland). The IC₅₀ values for the tested compounds are set forth under the heading “IC₅₀ A2K” in Table 2 below.

In addition, to further evaluate cytotoxic activity of the illustrative agents against cancer cell lines, an in vitro cytotoxicity assay was performed, essentially as described in Example 18 above. The compounds tested in the assay included Compounds 1, 2, 3, 4, 8, 26, 34, 42, 107 and 115, as set forth above in Table 1.

Briefly, tumor cell lines used were purchased from the American Type Culture Collection, and are identified as follows: Panc-1 (pancreas), MiaPaCa-2 (pancreas), MCF-7 (breast), HT-29 (colon), U2-OS (osteosarcoma), OVCAR-3 (ovary), HepG2 (hepatocellular carcinoma) and TT (medullary thyroid), PC-3 (prostate) and A549 (lung). The assay utilized the Cell-Titer-Glo Non-Radioactive Cell Proliferation Assay (Promega Corp., Madison, Wis.). First the cells were cultured in RPMI 1640 medium (Cat# 21870-076, Invitrogen Corporation) supplemented with 300 mg/L L-glutamine, 100 units/ml penicillin, 100 μg/ml streptomycin and 10% fetal bovine serum. All the cell lines were incubated in a humidified incubator at 37° C. with 5% CO₂ atmosphere.

Cells were plated at a density of 2000 to 10000 cells per well, depending on their growth rate, in 0.09 mL medium on day 0 in 96-well Microlite TCT microtiter plates (7418, Thermo Labsystems, Franklin, Mass.). On day 1, 10 μL of serial dilutions of the individual compounds were added to the plates in replicates of 3. After incubation for 4 days at 37° C. in a humidified incubator, the cells were lysed in the Cell-Titer-Glo reagent, which also contains luciferase enzyme. The luciferase reaction utilizes ATP released from lysed cells to produce light, the intensity of which is linearly related to the amount of ATP. Thus, the amount of light produced is a reflection of the number of cells remaining in the well after drug treatment. This luminescence was measured using a Luminoskan luminometer (Thermo Electron Corp., Vantaa, Finland) Data were expressed as the percentage of survival of control cells calculated from the luminescence corrected for background. The surviving percent of cells was determined by dividing the mean luminescence values of the treated wells by the mean luminescence values of the control and multiplying by 100.

The calculated IC₅₀ values for the each of the tested compounds against the various cancer cell lines are shown in Table 2 below:

TABLE 2
Cell-based IC₅₀ values (uM)

| Comp # | IC₅₀ A2K | Panc-1 | MiaPaCa-2 | MCF-7 | HT-29 | U2-Os | OVCAR-3 | HepG2 | PC-3 | A549 | TT |
|---|---|---|---|---|---|---|---|---|---|---|---|
| 1 | 0.49 | 37.50 | 18.61 | 14.60 | 5.01 | 13.15 | 13.07 | 25.97 | 52.29 | 53.78 | 11.00 |
| 2 | 0.005 | 197.66 | 172.64 | 125.59 | 93.60 | 71.72 | 29.40 | 294.69 | 164.04 | 194.06 | 63.97 |
| 3 | 0.074 | 35.76 | 22.11 | 208.85 | 29.80 | 20.83 | 47.98 | 49.55 | 47.29 | 37.03 | 13.20 |
| 4 | 0.023 | 136.13 | 85.19 | 248.94 | 103.20 | 36.29 | 79.04 | 88.00 | 159.70 | 72.69 | |
| 8 | 0.018 | | | 83.10 | 27.31 | | 82.37 | 81.79 | | | |
| 26 | 0.317 | 138.89 | 8.92 | 14.66 | 176.45 | 6.95 | 156.29 | 4.38 | 22.31 | 129.83 | |
| 34 | 2.09 | 125.32 | 9.82 | 76.91 | 138.37 | 27.01 | 110.86 | 81.93 | 21.93 | 186.24 | 99.13 |
| 42 | 4.97 | 216.22 | 182.29 | 4.62 | 139.94 | 41.33 | 85.06 | 98.49 | 189.38 | 160.03 | 128.76 |
| 107 | 0.586 | 300.00 | 107.08 | >300 | >300 | 8.70 | 175.57 | 172.93 | 2.33 | 51.52 | 146.65 |
| 115 | 0.743 | 49.82 | 55.35 | 87.84 | 97.42 | 50.37 | 174.69 | 57.93 | 75.68 | 149.05 | 38.63 |

### Example 21

### Chemical Synthesis of Further Illustrative Kinase Inhibitors

¹H NMR spectra were recorded on a Varian 400 spectrometer, using the solvent as an internal standard. Chemical shifts are expressed in ppm (δ). Proton magnetic resonance chemical shift values were measure in deuterated CDCl3 or DMSO-d6 unless otherwise stated. ESI mass spectra (MS) were obtained on a VG-Quattro II and PE-SEIEX (API) mass spectrometer. Thin-layer chromatography was performed on Merck Kieselgel silica 60 plates coated with 250 μm layer with fluorescent indicator. Components were visualized by UV light (λ=254 nm) and or by iodine vapor. Flash column chromatographic separations were carried out on 70-230 mesh 60 Å silica gel and on CombiFlash companion (Teledyne ISCO) using RediSep flash columns. All the solvents used were best grade anhydrous obtained from Aldrich. Analytical HPLC was performed on a Waters Breeze system using the following and quoted as retention time (RT) in minutes. The column used was symmetry C18 5 μm, 4.6×150 mm column (WAT045905). All experiments dealing with moisture-sensitive compounds were conducted under dry nitrogen or argon. Starting materials, unless otherwise specified, were commercially available (Aldrich, Fluka, Lancaster and TCI) and of the best grade and were used without further purification. Organic solutions, where applicable, were dried over anhydrous Na₂SO₄ and evaporated using a Yamamto RE500 rotary evaporator at 15-20 mmHg. Certain synthetic examples below make reference to illustrative synthesis Schemes 4-6 above.

### Example 22

### Synthesis of 4-chloro-1,2-dimethoxy-benzene 2 (Scheme 4)

In a 500 mL three-necked flask with a thermometer, CaCl₂ guard tube and dropping funnel, 25 g of the reactant 1 (veratrol) was introduced at 0° C., followed by drop by drop addition of 24.42 g (14.54 mL) of sulfuryl chloride. When the addition was completed, the reaction mixture was brought to RT after 1 hour and distilled under reduced pressure at 125-130° C. and the obtained colorless/pale-yellow oil was collected and dried to give the product 2. (Yield: 27.8 g (88% as yellow color liquid); bp:125° C.)

### Example 23

### Synthesis of 1-chloro-4,5-dimethoxy-2-nitrobenzene 3 (Scheme 4)

In a 500 mL three-necked flask with a thermometer and dropping funnel, was charged 27.8 g (1 eq) of 4-chloro-1,2-dimethoxybenzene 2 followed drop by drop addition of 30.43 g (3 eq, 20.4 ml) of nitric acid (d: 1.4) without the temperature being allowed to exceed to 25° C. When the addition was completed the reaction mixture was allowed to stand 1.5 h and the obtained compound 3 (1-chloro-4,5-dimethoxy-2-nitrobenzene) was treated with water and the precipitated yellow solid was filtered and washed with water and dried. (Yield: 31.3 g (89.4% as yellow solid); mp: 103-105° C.)

### Example 24

### Synthesis of ethyl 2-cyano-2-(4,5-dimethoxy-2-nitrophenyl)acetate 4 (Scheme 4)

Potassium tert-butoxide (30.1 g) was charged into an ice cold solution of ethyl cyanoacetate (29.4 mL) in THF (250 mL) and was stirred for 15 min. To the white suspension, the reactant 3 (30.10 g) was added and the reaction mixture was heated to reflux for 24 hrs. After completion of the reaction, the cooled reaction mixture was poured into water and extracted into diethyl ether and also DCM. The combined solvents were evaporated. The obtained crude MP-4 with starting material was purified by a flash column prior to using in next step. The solvent system used was 3:7 DCM/hexane. The eluent was changed to DCM and 10% methanol after the recovery of complete starting material. The slow moving spot was the compound confirmed from TLC. (Yield: 31.3%)

### Example 25

### Synthesis of ethyl 2-amino-5,6-dimethoxy-1H-indole-3-carboxylate 5 (Scheme 4)

A solution of ethyl 2-cyano-2-(4,5-dimethoxy-2-nitrophenyl)acetate 4 (9.5 g; 1 eq) in AcOH (50 mL) was reacted with Zn dust (8.44 g; 4 eq) by heating at 65° C. for 12 hours. The reaction mixture was cooled and filtered through a filter aid (celite) and was washed well with MeOH and the filtrate was concentrated to a residue, was treated with water and extracted with dichloromethane. The residue was purified by column chromatography using DCM initially followed by addition of 5% MeOH. The first elution recovered the starting material 4 and the second elution obtained the compound 5. (Yield: crude 7.1 g (purified product was 4.4 g as brown solid; 51.3%); mp: 164-166° C.)

### Example 26

### Synthesis of 6,7-dimethoxy-3H-pyrimido[4,5-b]indol-4(9H)-one 6 (Scheme 4)

A solution of ethyl 2-amino-5,6-dimethoxy-1H-indole-3-carboxylate 5 (4.4 g; 1 eq), NaOMe g (900 mg), and formamide (50 ml) was heated under N₂ at 220° C. for 1.5 hrs. The solution was cooled and stored at RT overnight and water was added. The solid separated out from the formamide was filtered and washed with water and dried to obtain compound 6 as a dark brown solid which was purified by column chromatography using DCM and 5% MeOH. (Yield: 2.8 g (68.6%, as half white); mp: 296-298° C.)

### Example 27

### Synthesis of 4-Chloro-6,7-dimethoxy-9,9a-dihydro-4-aH-pyrimido[4,5-b]indole 7 (Scheme 4)

A suspension of compound MP-6 (2.8 g), POCl₃ (20 mL) and p-dioxane (65 mL) was heated at reflux for 8 hrs. The obtained mixture was filtered and concentrated. The crude product was purified by column chromatography using 1% MeOH/DCM to give compound 7 (4-Chloro-6,7-dimethoxy-9,9a-dihydro-4-aH-pyrimido[4,5-b]indole). The obtained 7 pure product was a pale yellow solid. (Yield: 2.2 g (73.1% as pale yellow solid); mp: 148-150° C.)

### Example 28

### Synthesis of 6,7-dimethoxy-4-(piperazin-1-yl)-9H-pyrimido[4,5-b]indole 8 (Scheme 4)

Compound 7 was dissolved in p-dioxane (50 mL) and piprazine (3.9 g) was added following the addition of pyridine (15 mL) under argon at RT. The reaction mixture was heated to reflux for 12 hours and it was cooled. The solvents were removed under vacuum and the obtained crude product as purified by flash column chromatograph using DCM and 10% MeOH solvent system. The compound 8 obtained after purification was half white solid. (Yield: 3.9 g (66.6% as half white solid); mp: 185° C.)

### Example 29

### Synthesis of N-(4-fluorophenyl)-4-nitrobenzenesulfonamide 12 (Scheme 4)

A mixture of 15 ml of acetic acid, 5 g (22.56 mmol) of 4-Nitrobenzenesulfonylchloride, 2.507 g of 4-fluoroaniline and 3.70 g (45.1 mmol) of anhydrous sodium acetate was refluxed for 4 h and then to the reaction mixture 100 ml of boiling water was added. After the mixture was cooled, the precipitate was filtered off, washed with water and dried to yield 12. (Yield: 5.98 g; 89% as yellow solid)

### Example 30

### Synthesis 4-amino-N-(4-fluorophenyl)benzenesulfonamide 13 (Scheme 4)

The compound 12 and 10% Pd/C in ethanol was hydrogenated for 12 h. After the removal of the catalyst by filtration through celite, the solvents were evaporated and dried. The crude product was pure enough for next step. (Yield: 3.46 g; 88% as pale orange solid)

### Example 31

### Synthesis of N-(4-fluorophenyl)-4-isothiocyanatobenzenesulfonamide 14 (Scheme 4)

Compound 13 was dissolved in 10 mL of DCM and was added to a solution of 1.35 g of CaCO3, and 1.031 mL of thiophosgene dissolved in 10 mL of water. The reaction mixture was stirred overnight. The crude product was extracted into DCM and washed with water, the solvents were evaporated and the crude material was purified by purified by CombiFlash Companion using DCM solvent system (12 g normal phase RediSep Flash column with run time 40 min at flow 26 mL/min) to obtain the pure 14. (Yield: 2.93 g (70.3% half white solid)

### Example 32

### Synthesis of 4-nitro-N-(4-(trifluoromethyl)phenyl)benzenesulfonamide 9 (Scheme 4)

A mixture of 15 ml of acetic acid, 5 g (22.56 mmol) of 4-Nitrobenzenesulfonylchloride, 3.64 g of 4-(Trifluoromethyl)aniline and 3.70 g (45.1 mmol) of anhydrous sodium acetate was refluxed for 4 h and then to the reaction mixture 100 ml of boiling water was added. After the mixture was cooled, the precipitate was filtered off, washed with water and dried to yield 9. (Yield: 2.1 g; 26.9%)

### Example 33

### Synthesis 4-amino-N-(4-(trifluoromethyl)phenyl)benzenesulfonamide 10 (Scheme 4)

The compound SGI-488 and 10% Pd/C in Ethanol was hydrogenated for 12 h. After the removal of the catalyst by filtration through celite, the solvents were evaporated and dried. The crude product 10 was pure enough for next step. (Yield: 0.163 g; 44.6%).

### Example 34

### Synthesis of 4-isothiocyanato-N-(4 (trifluoromethyl)phenyl)benzene-sulfonamide 11 (Scheme 4)

Compound 10 was dissolved in 10 mL of DCM and was added to a solution of 0.058 g of CaCO3, and 0.044 M mL of thiophosgene dissolved in 10 mL of water. The reaction mixture was stirred overnight. The solvents were evaporated and the crude material was purified by CombiFlash Companion using DCM solvent system (12 g normal phase RediSep Flash column with run time 40 min at flow 26 mL/min) to obtain the pure 11. (Yield: 0.063 g; 60.4% as half-white solid)

### Example 35

### Synthesis of 4-(6,7-dimethoxy-9H-pyrimido[4,5-b]indol-4-yl)-N-(4-(N-(4-fluorophenyl)sulfamoyl) phenyl)piperazine-1-carbothioamide (Cmpd 2-3; Scheme 4)

To a stirred solution of compound 8 (50 mg) in DCM (25 mL) was added the compound 14 (49 mg) followed by the addition of pyridine (0.1 ml). The resulting reaction mixture was stirred at RT for 12 hours. After the completion of reaction, the solvents were evaporated. The crude product was purified by CombiFlash Companion using 1% MeOH in DCM solvent system (4 g normal phase RediSep Flash column at a flow of 18 mL/min). (Yield: 0.049 g; 49.4% as white solid)

### Example 36

### Synthesis of 4-(6,7-dimethoxy-9H-pyrimido[4,5-b]indol-4-yl)-N-(4-(N-(4-trifluoronethylhenyl)sulfamoyl)phenyl)piperazine-1-carbothioamide: (Cmpd 2-4; Scheme 4)

To a stirred solution of compound 8 (50 mg) in DCM (25 mL) was added the compound 11 (57 mg) followed by the addition of pyridine (0.1 mL). The resulting reaction mixture was stirred at RT for 12 hours. After the completion of reaction, the solvents were evaporated. The crude product was purified by CombiFlash Companion using 1% MeOH in DCM solvent system (4 g normal phase RediSep Flash column at flow 18 mL/min). (Yield: 0.019 g; 17.7% as white solid)

### Example 37

### Synthesis of ethyl 2-cyano-2-(5-methoxy-2-nitrophenyl)acetate 15 (Scheme 5)

Potassium tert-butoxide 15 g (2 eq) was charged into an ice cold solution of ethyl cyanoacetate 18 g (17 mL, 2 eq)) in THF (200 mL) and was stirred for 15 min. To the white suspension, the compound 59 (1-chloro-5-methoxy-2-nitrobenzene) 15 g (1 eq) was added and the reaction mixture was heated to reflux for 24 h to 4 days. The cooled reaction mixture was poured into water and extracted into ether and the solvent was evaporated. The obtained compound crude ethyl 2-cyano-2-(5-methoxy-2-nitrophenyl)acetate MP-60 with starting material (7:3 ratio from TLC) was purified by flash column prior to using in next step. The solvent system was 3:7 chloroform/hexane. The eluent was changed to chloroform and 10% methanol after the complete starting material was recovered. The slow moving spot was the compound 15 confirmed from TLC. (Yield: 7.6 g; 36.0% as dark yellow oil)

### Example 38

### Synthesis of ethyl 2-amino-6-methoxy-1H-indole-3-carboxylate 16 (Scheme 5)

A solution of ethyl 2-cyano-2-(5-methoxy-2-nitrophenyl)acetate 15 (1.4 g; 1 eq) in AcOH (50 mL) was reacted with Zn dust (1.38 g; 4 eq) by heating at 65° C. for 2 hours and subsequently treated with additional Zn powder. After heating for another 10 hrs, the brown mixture was filtered through a filter aid and was washed well with AcOH and the filtrate was concentrated to a residue. The residue (brown in color) was purified by column chromatography using DCM initially followed by addition of 5% MeOH in DCM. (Yield: 0.96 g; 77% as greenish-brown solid)

### Example 39

### Synthesis of 4-Chloro-7-methoxy-9,9a-dihydro-4-aH-pyrimido[4,5-b]indole 18 (Scheme 5)

A suspension of compound 17 (85 mg), POCl₃ (10 mL) and p-dioxane (10 mL) was heated at reflux for 6 hrs. The reaction mixture was cooled and the solvents were evaporated. The crude product was purified by column chromatography using 1% MeOH/DCM to give compound 18. (Yield: 0.21 g; 64.5% as pale yellow solid)

### Example 40

### Synthesis of 7-methoxy-4-(piperazin-1-yl)-9H-pyrimido[4,5-b]indole 19 (Scheme 5)

Compound 18 was dissolved in p-dioxane (10 mL) and piprazine (0.1 g) was added following the addition of pyridine (0.31 mL) under argon at RT. The reaction mixture was heated to reflux for 12 hours and then was cooled. The solvents were removed under vacuum and the crude product obtained as purified by flash column chromatography using a DCM and 10% MeOH solvent system.

The compound 19 was obtained after purification. (Yield: 0.63 g; 60.4% as half white solid)

### Example 41

### Procedure for the synthesis of 4-Nitro-N-p-tolyl-benzenesulfonamide 20 (Scheme 5)

A mixture of 10 ml of acetic acid, 4 g (18.05 mmol) of 4-Nitrobenzenesulfonylchloride, 1.934 g of p-Toluidine and 2.96 g (36.1 mmol) of anhydrous sodium acetate was refluxed for 4 h and then to the reaction mixture 100 ml of boiling water was added. After the mixture was cooled down, the precipitate was filtered off, washed with water and dried to yield 20. (Yield: 3.96 g; 75% as white solid)

### Example 42

### Synthesis 4-Nitro-N-p-tolyl-benzenesulfonamide 21 (Scheme 5)

The compound 20 and 10% Pd/C in Ethanol was hydrogenated for 6 h. After the removal of catalyst by filtration through celite, the solvents were evaporated and dried. The crude product was pure enough for the next step. (Yield: 0.79 g; 88% as half-white solid)

### Example 43

### Synthesis of 4-Isothiocyanato-N-p-tolyl-benzenesulfonamide 22 (Scheme 5)

Compound 21 was dissolved in 10 mL of DCM and was added to a solution of 0.401 g of CaCO3 and 0.291 mL of thiophosgene dissolved in 10 mL of water. The reaction mixture was stirred overnight. The solvents were evaporated and the crude material was purified by CombiFlash Companion using DCM solvent system (12 g normal phase RediSep Flash column with run time 40 min at flow 26 mL/min) to obtain the pure 22. (Yield: 0.86 g; 74.1% as half-white solid)

### Example 44

### Synthesis of 4-(7-methoxy-9H-pyrimido[4,5-b]indol-4-yl)-N-(4-(N-(4-fluorophenyl)sulfamoyl) phenyl)piperazine-1-carbothioamide (Scheme 5; Compound 2-5)

To a stirred solution of compound 19 (38 mg) in DCM (25 mL) was added the compound 14 (32.6 mg) followed by the addition of triethylamin (0.1 ml). The resulting reaction mixture was stirred at RT for 2 hrs. After the completion of reaction, the solvents were evaporated. Combiflash (4 g, 4% MeOH/DCM isocratic) gave SGI-1097 as white solid (46 mg). (Yield: 0.046 g; 73.4% as white solid). Analysis: TLC: DCM:MeOH (9:1), NMR and Mass was confirmed. HPLC purity was checked.

### Example 45

### Synthesis of 4-(7-Dimethoxy-9H-pyrimido[4,5-b]indol-4-yl)-piperazine-1-carbothioic acid (4-p-tolylsulfamoyl-phenyl)-amide (Scheme 5; Compound 2-7)

To a stirred solution of compound 19 (100 mg) in DCM (25 mL) was added the compound 22 (107 mg) followed by the addition of triethylamine/5% DMSO. The resulting reaction mixture was stirred at RT for 12 hrs. After the completion of reaction, the solvents were evaporated. Combiflash (12 g, 70% Hexane to DCM 10 min, DCM 10 min, to 10% MeOH 60 min, at 60 min, changed to premixed 5% MeOH/DCM 10 min) gave 200 mg of pale yellow solid. (Yield: 0.2 g; 96% as white solid)

### Example 46

### Synthesis of 4-(2,5-dichloro-4-nitrophenyl)morpholine 23 (Scheme 6

Compound 2,5-dichloro-4-nitroaniline, Potassium carbonate and 2-bromoethy ether in Acetonitrile was heated to reflux for 17 hours. The cooled reaction mixture was evaporated and purified by Combiflash Companion using Hexane 85% and DCM solvent system (40 g normal phase RediSep Flash column with run time 100 min at flow 30 ml/min) gave the compound 23. (Yield: 2 g; 19.97% as pale yellow solid)

### Example 47

### Synthesis of ethyl 2-(4-chloro-5-morpholino-2-nitrophenyl)-2-cyanoacetate 24 (Scheme 6)

Potassium t-Butoxide (1.62 g) was charged into an ice cold solution of ethyl cyanoacetate (1.152 mL) in THF (50 mL) and was stirred for 15 min. To the white suspension, the compound 23 (2 g) was added after and the reaction minxture was heated to reflux for 4 days. The cooled reaction mixture was evaporated to dry and poured into water, and extracted into EtOAc and Ether. The solvent was dried and evaporated. The obtained compound was purified by Combiflash Companion using hexane 40% for 40 min, hexane 40%-0 for 40 min and DCM solvent system (12 g normal phase redisep Flash column at flow 25 ml/min). (Yield: 1.2 g; 47% as yellow oiliness solid)

### Example 48

### Synthesis of ethyl 2-amino-6-chloro-5-morpholinoindoline-3-carboxylate 25 (Scheme 6)

A solution of 24 (1.2 g) in AcOH was reacted with Zn dust (1.33 g) by heating at 65° C. for overnight. The reaction mixture was filtered through SiO₂ and washed with MeOH and filtrate was concentrated to afford compound 25. (TLC, Rf=0.28, 0.31) (Yield: 1.0 g; 90% as greenish solid)

### Example 49

### Synthesis of 7-chloro-6-morpholino-3H-pyrimido[4,5-b]indol-4(9H)-one 26 (Scheme 6)

A solution of 25, NaOMe and Formamide was heated under Argon at 220° C. for 1.5 hr. The solution was cooled and stored for 1 day. The solid separated out from the Formamide after the addition of water was filtered and washed with water and dried to obtain compound 26. The product was purified by Combiflash Companion using DCM and 1-8% MeOH solvent system. (Yield: 0.1 g; 10.69% as brown solid)

### Example 50

### Synthesis of 4-(4,7-dichloro-9H-pyrimido[4,5-b]indol-6-yl)morpholine 27 (Scheme 6)

A suspension of compound 26, POCl3 and p-dioxane was heated at reflux for 17 h. The obtained mixture was concentrated and co-evaporated with DCM. The crude product was purified by Combiflash Companion using DCM and 1-6% MeOH solvent system (4 g normal Phase redisep column with run time 90 min at flow 18 mL/min) eluted the compound 27. (Yield: 30 mg; 28.3% as yellow oiliness solid)

### Example 51

### Synthesis of 4-(7-chloro-4-(piperazin-1-yl)-9H-pyrimido[4,5-b]indol-6-yl)morpholine 28 (Scheme 6)

Compound 27 was dissolved in p-dioxane and piprazine was added following the addition of pyridine under argon at RT. The reaction mixture was heated to reflux for 12 hours and then cooled. The solvents were removed under vacuum and obtained crude product as purified by flash column chromatography using DCM and 7% MeOH solvent system. (TLC, Rf=0.1, 10% MeOH/DCM)

### Example 52

### Synthesis of 4-(7-chloro-6-morpholino-9H-pyrimido[4,5-b]indol-4-yl)-N-(4-(N-tolylsulfamoyl)phenyl)piperazine-1-carbothioamide (Scheme 6; Compound 2-9)

Pyridine was added to a solution of 28 and 8 in DCM at RT and stirred for 12 hours. The solvent was evaporated and purified using 40% Hexane and DCM. (Yield: 80 mg; 88% as yellow solid) (Analysis: TLC: 10% MeOH/DCM, NMR and Mass spectra confirmed the structure)

### Example 53

### Synthesis of 4-(7-chloro-6-morpholino-9H-pyrimido[4,5-b]indol-4-yl)-N-(4-(N-(4-fluorophenyl) sulfamoyl)phenyl)piperazine-1-carbothioamide (Scheme 6; Compound 2-10)

Pyridine was added to a solution of 28 and 14 in DCM at RT and stirred for 12 hours. The solvent was evaporated and purify using 40% Hexane and DCM.

### Example 54

### Inhibition of Aurora-2 Kinase Activity by Compounds 2-3, 02-5, 2-7, 2-4 and 2-9

Illustrative compounds 2-3, 2-5, 2-7, 2-4 and 2-9 were evaluated in an aurora-2 kinase inhibition assay. In this assay kinase activity was determined by quantifying the amount of ATP remaining in solution following a kinase reaction by measuring the light units (LU) produced by luciferase using a luminometer. Percent inhibition was determined for individual compounds by comparing luminometer readings of drug-treated reactions to controls containing no drug (DMSO control) and no Aurora-2 enzyme (ATP control) in the following equation:

$$Percent Inhibition = \frac{{LU}_{drug} - {LU}_{DMSO}}{{LU}_{ATP} - {LU}_{DMSO}} \times 100$$

In a 50 μl reaction, recombinant aurora-2 kinase produced in sf9 cells (Upstate, Lake Placid, N.Y.) was incubated at 30° C. for two hours with 62.5 μM Kemptide (Calbiochem, San Diego, Calif.), 3 μM ATP (Invitrogen, Carlsbad, Calif.) and kinase reaction buffer (40 mM Tris-HCl, 10 mM MgCl₂ and 0.1 μg/μl bovine serum albumin (BSA)). This reaction was carried out in the presence of drug substances, which had been previously diluted to desired concentrations in DMSO. After incubation, 50 μl of Kinase-Glo® (Promega, Inc., Madison, Wis.) solution was added to each reaction mixture and allowed to equilibrate for 10 minutes at room temperature. Kinase-Glo solution contains luciferase enzyme and luciferin, which react with ATP to produce light. Kinase activity is determined by quantifying the amount of ATP remaining in solution following the kinase reaction by measuring the light units (LU) produced by luciferase using a luminometer (Thermo-Electron, Vantaa, Finland).

The drug concentration at which 50% of aurora-2 kinase activity was inhibited (IC₅₀) was determined for each compound. The IC₅₀ for SGI-498 was 0.110 μM, SGI-2023 was 0.837 μM, SGI-1097 was 0.569 μM, SGI-1215 was 0.455 μM, and SGI-503 was 0.153 μM (Table 8).

TABLE 8
Aurora-2 Inhibition, IC₅₀ (μM)

| 2-3 | 2-9 | 2-5 | 2-7 | 2-4 |
|---|---|---|---|---|
| 0.110 | 0.837 | 0.569 | 0.455 | 0.153 |

### Example 56

### Compounds 2-3, 2-5, 2-7 and 2-9 Induce Cancer Cell Cytotoxicity

To evaluate cell killing of cancer cell lines, an in vitro cytotoxicity assay was performed. The tumor cell lines used were purchased from the American Type Culture Collection, and are identified as follows: Panc-1 (pancreas), MiaPaCa-2 (pancreas), MCF-7 (breast), HT-29 (colon), U2-OS (osteosarcoma), OVCAR-3 (ovary), HepG2 (hepatocellular carcinoma), PC-3 (prostate), and A549 (lung). The assay utilized the Cell-Titer-Glo Non-Radioactive Cell Proliferation Assay (Promega Corp., Madison, Wis.). First the cells were cultured in RPMI 1640 medium (Cat# 21870-076, Invitrogen Corporation) supplemented with 300 mg/L L-glutamine, 100 units/ml penicillin, 100 μg/ml streptomycin and 10% fetal bovine serum. All the cell lines were incubated in a humidified incubator at 37° C. with 5% CO₂ atmosphere.

Cells were plated at a density of 2000 to 10000 cells per well, depending on their growth rate, in 0.09 mL medium on day 0 in 96-well Microlite TCT microtiter plates (#7418, Thermo Labsystems, Franklin, Mass.). On day 1, 10 μL of serial dilutions of the individual compounds were added to the plates in replicates of 3. After incubation for 4 days at 37° C. in a humidified incubator, the cells were lysed in the Cell-Titer-Glo reagent, which also contains luciferase enzyme. The luciferase reaction utilizes ATP released from lysed cells to produce light, the intensity of which is linearly related to the amount of ATP. Thus, the amount of light produced is a reflection of the number of cells remaining in the well after drug treatment. This luminescence was measured using a Luminoskan luminometer (Thermo Electron Corp., Vantaa, Finland) Data were expressed as the percentage of survival of control cells calculated from the luminescence corrected for background. The surviving percent of cells was determined by dividing the mean luminescence values of the treated wells by the mean luminescence values of the control and multiplying by 100.

The calculated IC₅₀ values for compounds 2-3, 2-5, 2-7 and 2-9_for the following cell lines: Panc-1, MiaPaCa-2, MCF-7, HT-29, U2-OS, OVCAR-3, HepG2, PC-3, and A549 are listed in Table 9.

TABLE 9

IC₅₀ (μM)

| Cell Line | 2-3 | 2-5 | 2-7 | 2-9 |
|---|---|---|---|---|
| Panc-1 | 22.3 | 10.68 | 2.22 | 85.16 |
| MiaPaCa-2 | 7.19 | 6.89 | 12.02 | 26.92 |
| MCF-7 | 8.43 | 8.11 | — | 8.21 |
| HT-29 | 6.42 | 6.93 | 7.74 | 8.00 |
| U2-OS | 8.18 | 8.49 | 5.16 | 8.56 |
| OVCAR-3 | 9.14 | 6.79 | 0.51 | 7.61 |
| HepG2 | 9.02 | 8 | 16.16 | 25.56 |
| PC-3 | 8.78 | 7.68 | 5.28 | 8.25 |
| A549 | 23.02 | 6.73 | 7.33 | 19.45 |

### Example 57

### Compound 2-5 Inhibits Tumor Growth In Vivo

In order to evaluate the effectiveness of compound 2-5 against tumor cells in a living system, a xenograft study was performed in mice. 1×10⁷ HT-29 human colon cancer cells were injected subcutaneously into Nu/Nu athymic nude mice (Charles River Laboratories, Wilmington, Mass.). Tumor volume was measured according to the formula ((Width)²*Length)/2. Tumors were allowed to grow to approximately 200 mm³ in volume (Day 0), at which point mice were randomized to three groups. Eight mice were grouped to receive SGI-1097 as treatment, and two groups of eight would receive vehicle alone as a negative control. The SGI-1097 compound was formulated in the following vehicle: 10% EtOH; 30% PEG 300; 60% solution of 300 mg/ml 2-Hydroxypropyl-beta-cyclodextrin in water.

In the treatment group, each animal received 5 mg/kg of compound 2-5 in the aforementioned vehicle at a volume of 100 μl, dosed IP. The dosing schedule was QDx5 for three weeks. The negative control group received the following vehicle alone, dosed IV in the tail vain, on the same schedule as the treatment group: 5% Dimethyl Acetamide; 25% PEG 300; 70% solution of 300 mg/ml 2-Hydroxypropyl-beta-cyclodextrin in water.

Another negative control group was dosed PO with corn oil on the same dosing schedule. The tumor volumes for both the treated and untreated groups were measured twice a week for the three week dosing period, and for one week after the dosing period. No noticeable toxicity from drug or vehicle was noted through the duration of this study. Using this approach, compound 2-5 was found to be effective for inhibiting tumor growth in vivo.

### Example 58

### Inhibition of Axl Kinase Activity by Illustrative Compound 1-1

This example describes illustrative assays for determining the inhibitory activity of compounds of the invention against Axl kinase. The example further demonstrates that compound 1-1 is an active inhibitor of Axl kinase activity.

### A. Axl Kinase Inhibition Assay

One illustrative manner in which Axl kinase activity can be determined is by quantifying the phosphorylation of a known Axl substrate in an in vitro assay. In this assay kinase activity is determined by quantifying the amount of ATP remaining in solution following the kinase reaction by measuring the relative light units (RLU) produced by luciferase using a luminometer. Percent activity was determined for individual compounds by comparing luminometer readings of drug-treated reactions to controls containing no drug (RLU_{NO} Inhib) and no Aurora-2 enzyme (RLU_{NO} Kinase) in the following equation:

$$Percent Inhibition = \frac{{RLU}_{No Kinase} - {RLU}_{drug}}{{RLU}_{No Kinase } - {RLU}_{No Inhib}} \times 100$$

In a 50 μl reaction, 200 ng/μl of Axl kinase (BPS Bioscience Inc, San Diego Calif.) was incubated at 30° C. for two hours with shaking (360 rpm) with 10 μg/ml Poly (Glu:Tyr) substrate (BPS Bioscience Inc, Calif.), 3 μM ATP. (Invitrogen, Carlsbad, Calif.) and kinase reaction buffer (40 mM MOPS, 75 mM MgCl₂ and 0.1 mM EDTA). The value of 3 μM ATP was determined to be the Km (concentration at which the enzyme is working at 50% maximum velocity) for the amount of enzyme used in this assay. This reaction was carried out in the presence of drug substances, which had been previously diluted to desired concentrations in DMSO and water. After incubation, 50 μl of Kinase-Glo® (Promega, Inc., Madison, Wis.) solution was added to each reaction mixture and allowed to equilibrate for 10 minutes at room temperature. Kinase-Glo solution contains luciferase enzyme and luciferin, which react with ATP to produce light. Kinase activity is determined by quantifying the amount of ATP remaining in solution following the kinase reaction by measuring the relative light units (RLU) produced by luciferase using a luminometer (Thermo Electron Corporation, Vantaa, Finland).

### B. Cell-Based Axl Kinase Inhibitor Assays:

Cell culture-based assays can be used to evaluate the ability of compounds of the invention to inhibit one or more cellular activities, such as cancer cell growth and/or survival. Numerous cancer cell lines can be obtained from the American Type Culture Collection (ATCC) and other sources. Briefly, cells are seeded into 96-well, tissue-culture treated, opaque white plates (Thermo Electron, Vantaa, Finland), at between 5000 and 10000 cells per well, depending on the speed of cell proliferation, in 100 μl of appropriate growth medium (determined by the ATCC). Cells are then exposed to the appropriate concentration of drug or an equal amount of DMSO (drug diluent) and allowed to grow in its presence for 96 hours. Following this, 100 μl of Cell-Titer-Glo reagent (Promega, Inc., Madison, Wis.) is added to each well. Plates are then shaken for 2 minutes at room temperature to allow for cell lysis and incubated for 10 minutes to stabilize the luminescent signal. Similar to the Kinase-Glo assay reagent from Promega, this reagent contains both luciferase enzyme and its substrate luciferin. Luciferase, activated by ATP in the cell lysate, catalyzes the conversion of luciferin to oxyluciferin, a reaction which produces light. The amount of light produced is proportionate to the amount of ATP in the cell lysate, which is itself proportional to cell number and gives an index of cellular proliferation.

In order to detect specific inhibition of Axl enzyme in cell culture, a Western blot assay can also be performed. For this, cells known to over express Axl which have been treated with a potential Axl inhibitor are lysed with a buffer specific for the isolation and preservation of proteins (1% Nonidet P-40, 150 mM NaCl, 50 mM Tris pH 8.0, 5 mM EDTA, 1:500 Protease Inhibitor Cocktail III [Calbiochem], 100 mM NaF, 100 mM Sodium Orthovanadate). The protein concentration in these lysates is then quantified using the BCA Protein Assay Kit (Pierce). Known amounts of protein, e.g. 10 ug, are loaded onto 12% SDS-polyacrylamide gels and are subjected to reducing, denaturing SDS-PAGE. Electrophoresed proteins are transferred to a nitrocellulose membrane, which is then probed with any of the following antibodies: anti-NFκB, anti-Bcl-2, anti-caspase 3, anti-Akt and anti-phospho-Akt. All these are potentially downstream targets of Axl. Another analysis would involve co-immunoprecipitation against Tyrosine-phosphorylated Axl using an anti phospho tyrosine antibody followed by an antibody specifically against Axl

### C. Specific Activity Data

In an Axl kinase inhibition assay, performed essentially as described above, wherein the % enzyme activity was evaluated across a range of compound 1-1 drug concentrations, the IC₅₀ values for compound 1-1 was determined to be 9.8 μM.

In addition, compound 1-1 was tested for cell-based activity against various cancer cell-lines, essentially as described above, and the IC₅₀ values determined for compound 1-1 in these cell lines are summarized in Table 10 below. Values given for IC₅₀ represent the concentration in μM required to inhibit cell growth to 50% of untreated.

TABLE 10
Cell-based activity of Compound 1-1.

| | Compound 1-1, |
|---|---|
| Cell Line | IC₅₀ (μM) |
| Panc1 | 40.67 |
| MiaPaCa-2 | 66.59 |
| MCF-7 | 22.46 |
| HT-29 | 14.65 |
| U2-Os | 25.93 |
| OVCAR-3 | 24.97 |
| HepG2 | 7.83 |
| PC-3 | 28.37 |
| A549 | 19 |
| TT | 51.67 |
| MDA-MB-231 | 20.20 |

### Example 59

### Compound 2-3 is Specific for Aurora-2 Kinase

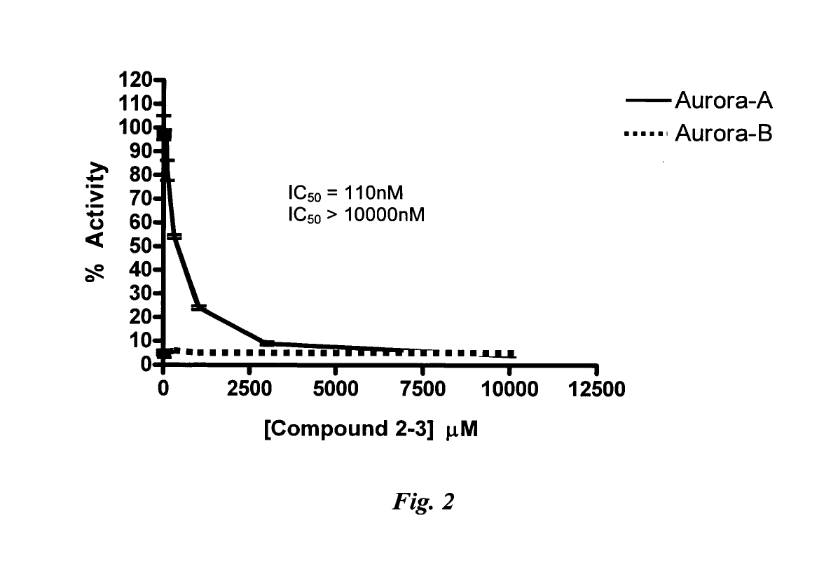

In order to determine the specificity of compound 2-3 within the Aurora kinase family, both Aurora-2 kinase (Aurora-A) and Aurora-1 kinase (Aurora-B) were examined for sensitivity to this molecule. These studies were performed using the SelectScreen kinase assay system (Invitrogen Corp.; Madison, Wis.) and gave rise to the results presented in FIG. 2.

Unexpectedly, compound 2-3 showed over 100-fold selectivity for Aurora-2 kinase over Aurora-1 kinase, making it unique and advantageous. This selectivity may result in reduced toxicity, because although inhibiting both family members is not expected to increase anti-tumor activity, it may increase general cytotoxic effects. In addition, the selectivity observed for compound 2-3 may result in improved tumor growth inhibition.

### Example 60

### Compound 2-3 Causes a Brief Cell Cycle Arrest in G2/M. Followed by Apoptosis in Tumor Cells

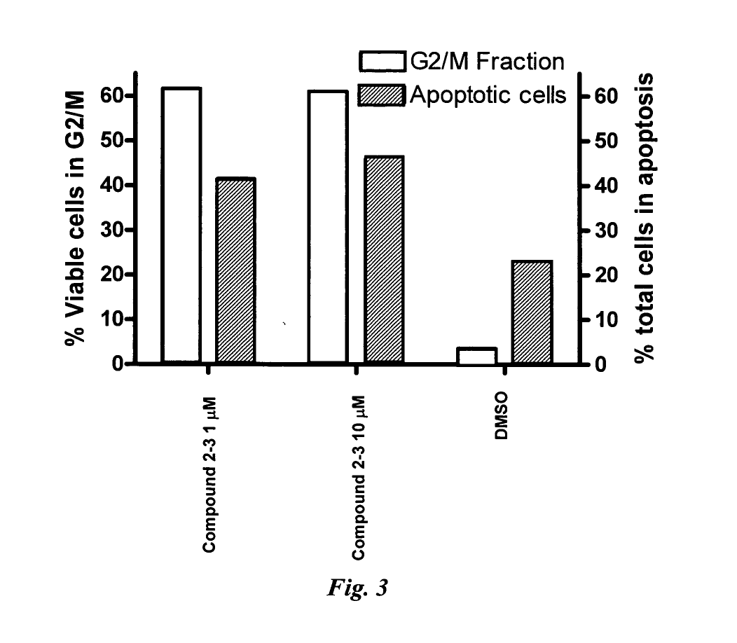

It is expected that a specific Aurora-2 kinase inhibitor would cause only brief cell cycle arrest at G2/M, which should be immediately followed by apoptosis. This is in contrast to Aurora-1 kinase inhibitors, which tend to cause a prolonged cell cycle arrest. compound 2-3 was examined for its ability to cause cell cycle arrest and apoptosis. Briefly, A549 non-small cell lung carcinoma cells were plated at 50000 cells/mL in a 24-well plate. Cells were synchronized with 4 μg/mL Aphidicolin for 16 hours, followed by a 24 hour synchronization with 1 μg/mL Hoechst 33342, to arrest the cells at the G2/M boundary. Cells were then released into regular medium with or without SGI-498 and allowed to grow for an additional 24 hours. This synchronization was necessary for visualization of the brief G2/M arrest caused by compound 2-3. Following this incubation, cells were stained for DNA content analysis using the Guava Cell Cycle Assay Kit (Guava Technologies, Hayward Calif.), according to manufacturer instructions. Cells were then analyzed with the Guava EasyCyte benchtop flow cytometer using the Guava Cell Cycle analysis protocol. Cells were gated according to DNA content into G0/G1, S, G2/M or apoptotic (sub-G1) categories. The results of this study are shown in FIG. 3.

Timepoints greater than 24 hours post-release did not show the G2/M arrest, as cells proceeded very quickly into apoptosis. Similarly, non-synchronized cells did not arrest long enough with compound 2-3 treatment to allow visualization of this arrest. These results are consistent with compound 2-3 being a potent and specific Aurora-2 kinase inhibitor.

### Example 61

### Compound 2-3 Treatment Causes an Increase in Histone H3 Serine 10 Phosphorylation, Consistent with Specific Aurora-2 Kinase Inhibition

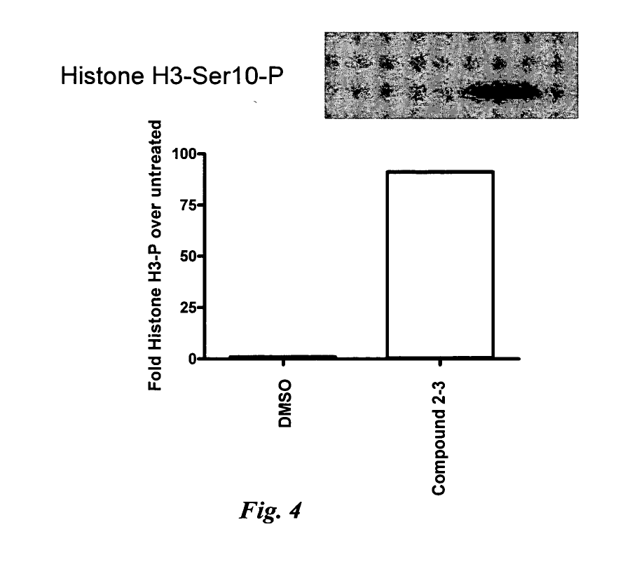

A hallmark of Aurora-2 kinase inhibition, which distinguishes it from Aurora-1 kinase inhibition, is an increase in Histone H3 phosphorylation on Serine 10, an activity which is controlled by Aurora-1 kinase. This is in contrast to the decrease in Histone H3 phosphorylation that has been observed with Aurora-1 kinase inhibition. Compound 2-3 was examined for its effect on Histone H3 phosphorylation, in order to further verify its specificity for Aurora-2 kinsase. Briefly, MiaPaCa-2 cells were grown in 25 cm² flasks to approximately 40% confluency, and then synchronized with Aphidicolin and Hoechst 33342 as described above. Cell were then released and treated with 30 M compound 2-3 in culture medium for 24 hours. Cells were then lysed, and total protein was quantified. 50 μg of total protein was electrophoresed and transferred to nitrocellulose, at which time Western Blot analysis for Histone H3 phospho-Serine 10 was performed. The results of this experiment are shown in FIG. 4. Untreated cells showed little to no Histone H3 phosphorylation, while compound 2-3-treated cells showed a pronounced phosphorylation signal at 24 hours. This signal quickly disappeared, as the cells began to undergo apoptosis, as discussed earlier. These data further support that compound 2-3 is a potent and specific inhibitor of Aurora-2 kinase, but does not affect Aurora-1 kinase.

### Example 62

### Further Synthesis of Representative Compounds

Additional compounds of the invention were prepared according to the general reaction scheme described below.

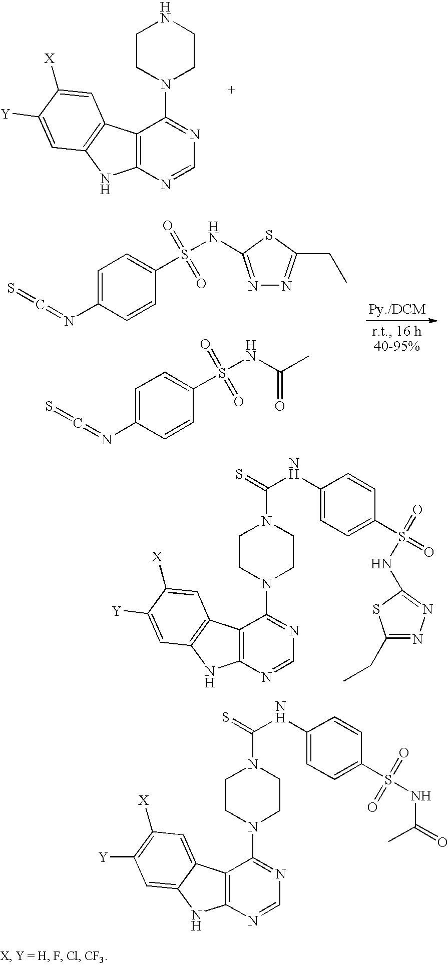

### 4-(6-Chloro-9H-pyrimido[4,5-b]indol-4-yl)-N-(4-(N-(5-ethyl-1,3,4-thiadiazol-2-yl)sulfamoyl)phenyl)piperazine-1-carbothoamide (2-12)

¹H-NMR (DMSO-d₆/400 MHz): 12.36 (s, 1H), 9.70 (s, 1H), 8.50 (s, 1H), 7.76 (s, 1H), 7.67 (d, J=8.2 Hz, 2H), 7.48 (m, 4H), 4.12 (m, 4H), 3.85 (m, 4H), 2.78 (q, J=7.5 Hz, 2H), 1.18 (t, J=7.5 Hz, 3H). MS (ES+, m/z): 613.9 (M⁺+1, 10.0).

### 4-(6-Chloro-7-trifluoromethyl-9H-pyrimido[4,5-b]indol-4-yl)-N-(4-(N-(5-ethyl-1,3,4-thiadiazol-2-yl)sulfamoyl)phenyl)piperazine-1-carbothoamide (2-15)

¹H-NMR (CD₃OD/400 MHz): 8.50 (s, 1H), 7.89 (d, J=8.9 Hz, 2H), 7.80 (d, J=8.9 Hz, 2H), 7.61 (s, 1H), 7.50 (s, 1H), 7.48 (s, 1H), 4.18 (s, 4H), 3.97 (s, 4H), 2.81 (q, J=7.5 Hz, 2H), 1.30 (t, J=7.5 Hz, 3H). MS (ES+, m/z): 681.9 (M⁺+1, 70.0).

### Example 63

### Activity of Additional Representative Compounds

IC₅₀ values were calculated, essentially as described above, for additional representative compound of the invention, the results of which are set forth in the Table below.

| Compd. Number | IC₅₀ μM |
|---|---|
| Cmpd 1-1 | 15.8 |
| Cmpd 1-3 | 18.7 |
| Cmpd 2-13 | 36.0 |
| Cmpd 2-14 | 1.4 |
| Cmpd 2-15 | 6.7 |

### Example 64

### Representative Compound 2-3 Sensitizes Lymphoma Cells to Taxol® Treatment

Granta-4 and Granta-22 lymphoma cell lines were treated with compound 2-3 or Taxol® for 48 hours, and IC₅₀ values determined as the concentration of compound required to reduce cell numbers to one-half of untreated controls. Cells were pretreated with the IC₅₀ of each agent (Taxol® or compound 2-3) for 24 hours, at which point the drug was removed, and fresh medium containing the other agent was added for a total of 48 hours. New IC₅₀ values were calculated for the second agent in an effort to determine whether or not pretreatment with one compound would increase sensitivity to the other. As set forth in the table below, compound 2-3 pretreatment led to an increased sensitivity to Taxol®, however Taxol® pretreatment appeared to be anergistic to compound 2-3 in this assay.

IC₅₀ Values (μM)

| Treatment | Granta-4 | Granta-22 |
|---|---|---|
| Compound 2-3 | 2.037 | 6.669 |
| Taxol ® | 0.301 | 2.724 |
| Compound 2-3, Taxol ® | 0.009 | 0.008 |
| Taxol ®, Compound 2-3 | 125.704 | 105.596 |

### Example 65

### Representative Compounds Show Antiprolilferative Activity in Tumor Cell Lines

Compound 1-1 and Compound 2-11 were tested in a cell proliferation assay to determine potency in a cell-based assay format. Briefly, MDA-MB-231 breast carcinoma cells were inoculated onto 96-well plates and incubated in the presence of compounds for 96 hours. Following incubation, cells were lysed and ATP content used as a surrogate for cell number, using the Cell-Titer-Glo Luminescent Cell Viability Assay Kit (Promega Corp.). IC₅₀ values, representing the concentration required to reduce cell number to 50% of untreated controls, were determined to be 20 uM for Compound 1-1 and 10 uM for Compound 2-11.

### Example 66

### Representative Compound Reduces Autophosphorylation of Axl in a Dose-Dependent Manner

Compound 2-11 was examined in a cell-based assay to determine its ability to reduce the activity of the Axl kinase enzyme in cell culture. Axl, like many receptor tyrosine kinases, autophosphorylates in response to growth factor stimulation. Briefly, MDA-MB-231 breast carcinoma cells were plated in 25 cm² tissue culture flasks and incubated in the presence of varying concentrations of Compound 2-11 for 48 hours. Following incubation, cells were lysed and the presence of phosphorylated Axl was detected using a commercially available sandwich ELISA assay kit (R&D Systems Inc.). Using this assay, Compound 2-11 was demonstrated to cause a reduction in phosphorylated Axl at low concentrations. For example, at concentrations as low as 10 uM, levels of phosphorylated Axl were reduced to less than 50% of those observed for untreated control samples.

### Example 67

### Synthesis of Further Representative Compounds

A. Synthesis of N-Benzoyl-4-isothiocyanato-benzenesulfonamide 29 (Scheme 7)

Compound sulfabenzamide (2 g) was dissolved in 15 mL of DCM and was added to a solution of 1.449 g of CaCO3 and 0.828 mL of thiophosgene dissolved in 15 mL of water. The reaction mixture was stirred overnight. The solvents were evaporated and the crude material was purified by column chromatography using 10% MeOH in DCM to obtain the pure 29 as white solid (1.93 g, 84%).

B. Synthesis of N-(4-(4-(6,7-dimethoxy-9H-pyrimido[4,5-b]indol-4-yl)piperazine-1-carbothioamido) phenylsulfonyl)benzamide (3-1) (Scheme 7)

To a stirred solution of compound 8 (0.060 g, 0.191 mmol) in DCM (20 mL) was added the compound 29 (0.061 g, 0.191 mmol) followed by the addition of pyridine (0.045 g, 0.046 mL, 0.574 mmol). The resulting reaction mixture was stirred at RT for 12 hours. After the completion of reaction, the solvents were evaporated. The crude product was purified by CombiFlash Companion using DCM and 5% MeOH in DCM solvent system (4 g normal phase RediSep Flash column with run time 40 min at flow 18 mL/min) yielded 0.049 g (40.5% as white solid).

### Example 68

### Aurora A Overexpression in Primary Malignancies*

Using a differential gene expression profiling tool (ASCENTA® System; Gene Logic), the expression profile of Aurora A in different cancer types was evaluated and the results are summarized in the table below. Illustrative compounds of the invention may be used in the treatment of such cancers.

| Cancer Type | Expression Level |
|---|---|
| Uterine/Cervix Squamous Cell Carcinoma | ++++ |
| Kidney Cancer (Wilm's Tumor) | ++++ |
| Small Cell Lung Carcinoma | +++ |
| Hepatocellular Carcinoma | ++ |
| Nonsmall Cell Lung Carcinoma | ++ |
| Melanoma | ++ |
| Infiltrating Ductal Breast Cancer | ++ |
| Infiltrating Lobular Breast Cancer | ++ |
| Lung Adenocarcinoma | ++ |
| Colorectal Cancer | ++ |
| Stomach Adenocarcinoma | ++ |
| Ovarian Adenocarcinoma | ++ |

++++ = 8-fold above respective normal tissue; +++ = 6-fold above respective normal tissue; ++ = 4-fold above respective normal tissue; + = 2-fold above respective normal tissue

### Example 69

### Growth Inhibition of Tumor Cells in Hollow Fiber Xenografts

Compound 2-3 was tested against three tumor types in athymic nude mice using the hollow fiber xenograft model. After incubation and growth overnight, drug-permeable fibers containing tumor cells were surgically implanted either intraperitoneally (IP) or subcutaneously (SC) and allowed to grow for 24-hours. After treatment with compound 2-3, fibers were removed and tumor cell growth was measured using the MTT assay. A549, Panc-1 or HT-29 cells were injected into separate fibers and each mouse received a fiber containing each cell line both SC and IP totaling six fibers per mouse. Compound 2-3 at doses of 20 mg/kg and 40 mg/kg administered by intravenous injection was most effective against Panc-1 fibers implanted SC (58.7% and 60.9% growth, respectively) and HT-29 fibers implanted IP (53.8% and 73.4%, respectively).

### Example 70

### Growth Inhibition of HT-29 Colorectal Xenograft

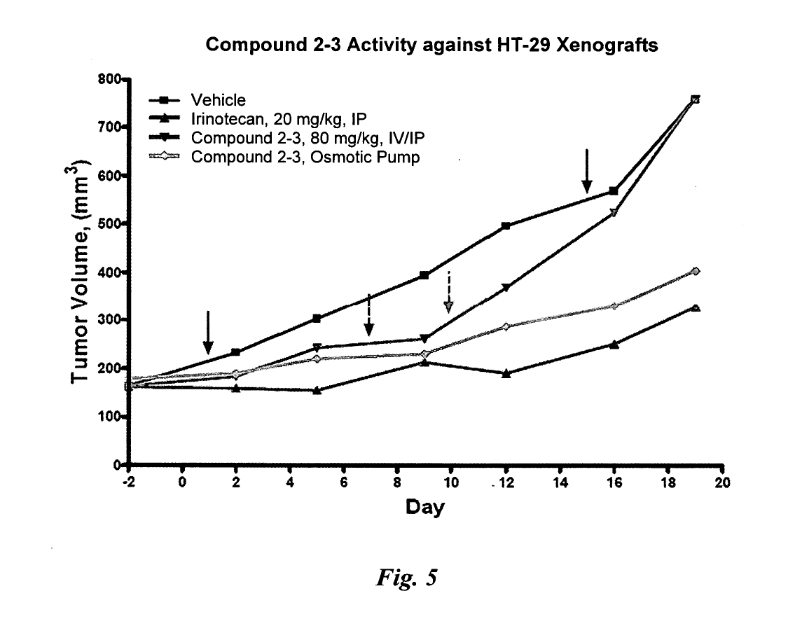

This study examined the effect of route of administration on tumor growth inhibition. Tumor bearing mice were treated with compound 2-3 intraperitoneally (ALZET osmotic pumps, delivering 25 mg/kg/day over 14 days) or intravenously (80 mg/kg/d on Days 1, 4 and 7). Because of difficulty in maintaining tail vein patency, this group of mice received subsequent dose intraperitoneally on Days 10, 11, 12 and 15. Compound 2-3 delivered continuously via the osmotic pump decreased tumor growth rate to 10.75 mm³/d, compared with vehicle-treated tumor growth rate of 28.28 mm³/d (see FIG. 5). Treated/Control ratio was 38%. Administration of compound 2-3 bolus by the IV route had a similar result as the pump up through Day 9. However, after dosing was switched to IP, this effect was lost.

### Example 71

### Growth Inhibition of DU145 Prostate Cancer Xenograft

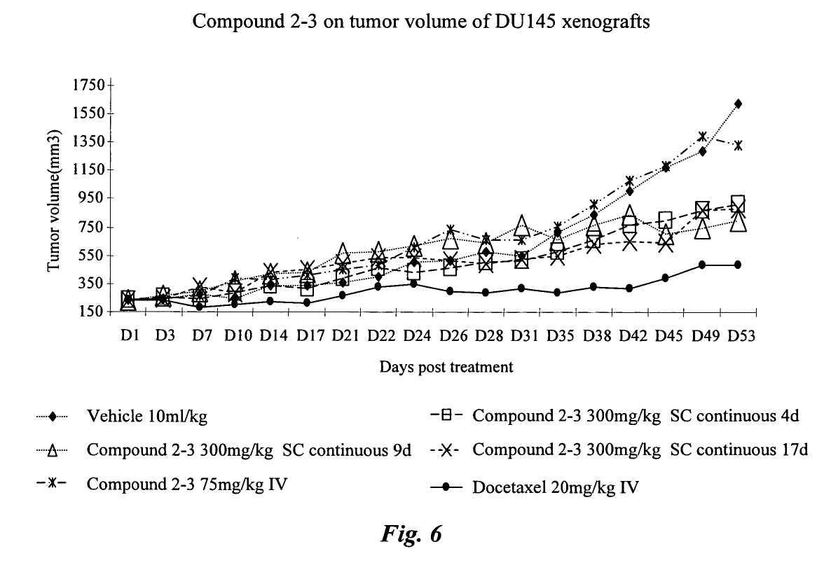

In this study, DU145 carcinoma model was used to evaluate the anti-tumor efficacy of compound 2-3. The murine xenograft model was established by inoculating 2×10⁶ cells into each Balb/c nude mouse via s.c. injection. When the average tumor size reached ˜100 mm³, the mice were randomly divided into different experimental groups based on tumor size. Mice were treated according to predetermined treatment regimen as shown in the figure below. Subcutaneous administration of compound 2-3 was conducted via an Alzet osmotic pump. On day 30 a second cycle of dosing was initiated. Repeat dose of compound 2-3 seemed to be well tolerated in mice, as indicated by the lack of weight loss throughout the study. Compound 2-3 led to a reduction in tumor growth in this model and was more effective when administered subcutaneously than intravenously (See FIG. 6).

Any U.S. patents, U.S. patent application publications, U.S. patent applications, foreign patents, foreign patent applications and non-patent publications referred to in this specification and/or listed in the Application Data Sheet are incorporated herein by reference, in their entirety.

From the foregoing it will be appreciated that, although specific embodiments of the invention have been described herein for purposes of illustration, various modifications may be made without deviating from the spirit and scope of the invention. Accordingly, the invention is not limited except as by the appended claims.

### REFERENCES

1. Keen, N. and S. Taylor, Aurora-kinase inhibitors as anticancer agents. Nat Rev Cancer, 2004. 4(12): p. 927-36.
2. Kimura, M., et al., Cell cycle-dependent expression and spindle pole localization of a novel human protein kinase, Aik, related to Aurora of Drosophila and yeast Ipl1. J Biol Chem, 1997. 272(21): p. 13766-71.
3. Warner, S. L., et al., Targeting Aurora-2 kinase in cancer. Mol Cancer Ther, 2003. 2(6): p. 589-95.
4. Warner, S. L., et al., Identification of a lead small-molecule inhibitor of the Aurora kinases using a structure-assisted, fragment-based approach. Mol Cancer Ther, 2006. 5(7): p. 1764-73.
5. Warner, S. L., et al., Comparing Aurora A and Aurora B as molecular targets for growth inhibition of pancreatic cancer cells. Mol Cancer Ther, 2006. 5(10): p. 2450-8.
6. Carmena, M. and W. C. Earnshaw, The cellular geography of aurora kinases. Nat Rev Mol Cell Biol, 2003. 4(11): p. 842-54.
7. Hauf, S., et al., The small molecule Hesperadin reveals a role for Aurora B in correcting kinetochore-microtubule attachment and in maintaining the spindle assembly checkpoint. J Cell Biol, 2003. 161(2): p. 281-94.
8. Kimura, M., et al., Identification and characterization of STK12/Aik2: a human gene related to aurora of Drosophila and yeast IPL 1. Cytogenet Cell Genet, 1998. 82(3-4): p. 147-52.
9. Sasai, K., et al., Aurora-C kinase is a novel chromosomal passenger protein that can complement Aurora-B kinase function in mitotic cells. Cell Motil Cytoskeleton, 2004. 59(4): p. 249-63.
10. Chen, H. L., et al., Overexpression of an Aurora-C kinase-deficient mutant disrupts the Aurora-B/INCENP complex and induces polyploidy. J Biomed Sci, 2005. 12(2): p. 297-310.
11. Araki, K., et al., High expression of Aurora-B/Aurora and IpII-like midbody-associated protein (AIM-1) in astrocytomas. J Neurooncol, 2004. 67(1-2): p. 53-64.
12. Bischoff, J. R., et al., A homologue of Drosophila aurora kinase is oncogenic and amplified in human colorectal cancers. Embo J, 1998. 17(11): p. 3052-65.
13. Katayama, H., W. R. Brinkley, and S. Sen, The Aurora kinases: role in cell transformation and tumorigenesis. Cancer Metastasis Rev, 2003. 22(4): p. 451-64.
14. Mahadevan, D., D. J. Bearss, and H. Vankayalapati, Structure-based design of novel anti-cancer agents targeting aurora kinases. Curr Med Chem Anti-Canc Agents, 2003. 3(1): p. 25-34.
15. Meraldi, P., R. Honda, and E. A. Nigg, Aurora kinases link chromosome segregation and cell division to cancer susceptibility. Curr Opin Genet Dev, 2004. 14(1): p. 29-36.
16. Rojanala, S., et al., The mitotic serine threonine kinase, Aurora-2, is a potential target for drug development in human pancreatic cancer. Mol Cancer Ther, 2004. 3(4): p. 451-7.
17. Vankayalapati, H., et al., Targeting aurora2 kinase in oncogenesis: a structural bioinformatics approach to target validation and rational drug design. Mol Cancer Ther, 2003. 2(3): p. 283-94.

We claim:

1. A compound having the following structure (I): 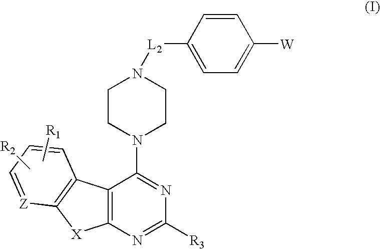
including stereoisomers and pharmaceutically acceptable salts thereof, wherein:
X is NH, S or O;
Z is CH or N;
R₁ and R₂ are the same or different and are independently hydrogen, hydroxyl, halo, —CN, —NO₂, —NH₂, —R, —OR, —SCH₃, —CF₃, morpholine, —C(═O)OR, —OC(═O)R, where R is alkyl or substituted alkyl; or —O(CH₂)ₙ—Rₓ, where n is 2-4 and Rₓ is N-methylpiperazine, morpholine or 2-methylpyrrolidine.
R₃ is hydrogen, —NH₂, alkyl, —CN, or —NO₂, or R₃ is -L₃-Cycl₃ wherein L₃ is a direct bond, —S— or —NH—, and CyCl₃ is a carbocycle, substituted carbocycle, heterocycle or substituted heterocycle;
L₂ is —C(═S)NH—,
W is: 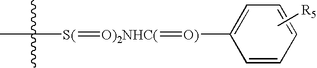
or —S(═O)₂NHR_{y}, where R_{y} is 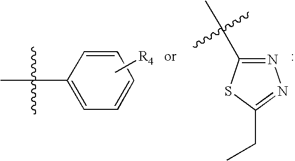
R₄ is alkyl, halo, or haloalkyl; and R₅ is hydrogen, alkyl, alkoxy, haloalkyl, halo, hydroxyl, or substituted alkoxy.

2. The compound of claim 1, where X is NH and Z is CH.

3. The compound of claim 1, where w is S(═O)₂NHR_{y}, where R_{y} is 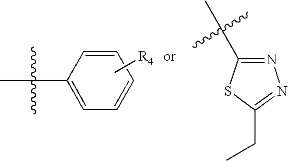
and where R₄ is alkyl, halo or haloalkyl.

4. The compound of claim 1, where R₁ and R₂ are selected from hydrogen, —OR, halo, haloalkyl or morpholine, where R is C₁-C₃ alkyl.

5. The compound of claim 1, where R₃ is hydrogen.

6. The compound of claim 2, where R₁ and R₂ are selected from hydrogen, —OCH₃, halo, CF₃, and morpholine, R₃ is hydrogen, and w is —S(═O)₂NHR_{y}, where R_{y} is: 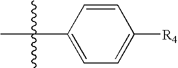
where R₄ is alkyl, halo or haloalkyl.

7. The compound of claim 1, where X is NH, Z is CH, L₂ is —C(═S)NH—, R₃ is hydrogen, and the compound has the following structure (V): 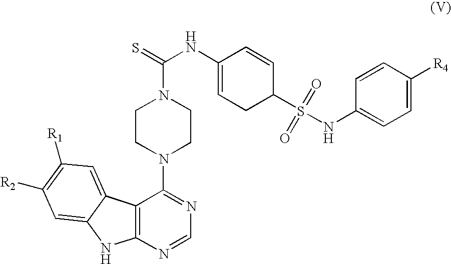
where R₁ and R₂ are selected from hydrogen, —OR, —OH, halo, haloalkyl or morpholine, where R is C₁-C₃ alkyl; and where R₄ is alkyl, halo or haloalkyl.

8. The compound of claim 7, where R₄ is CH₃, F or CF₃.

9. The compound according to claim 7, selected from:

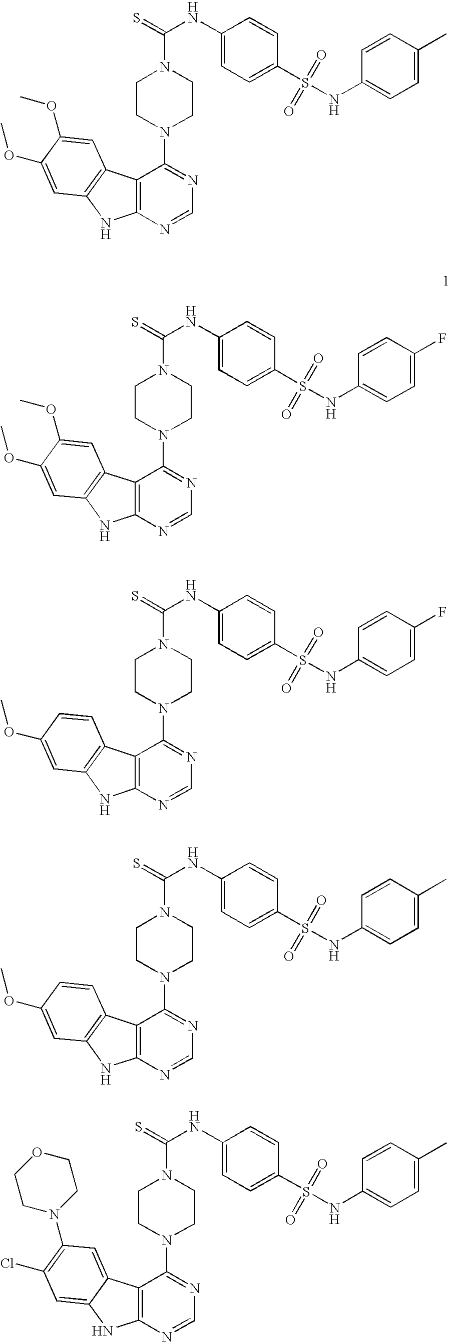

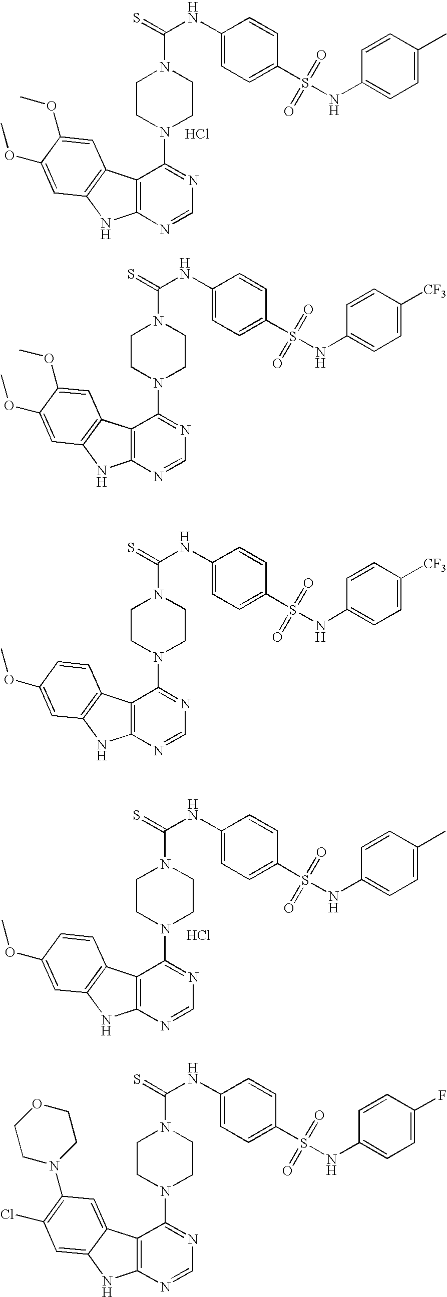

10. The compound of claim 7 having the following structure:

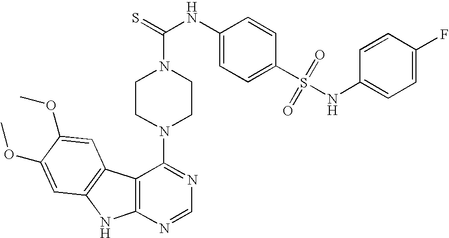

11. The compound of claim 1, where X is NH, Z is CH, L₂ is —C(═S)NH—, R₃ is hydrogen, and the compound has the following structure (VI): 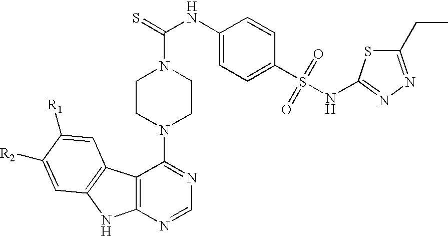
where R₁ and R₂ are selected from hydrogen, —OR, —OH, halo, haloalkyl or morpholine, where R is C₁-C₃ alkyl.

12. The compound according to claim 11, selected from:

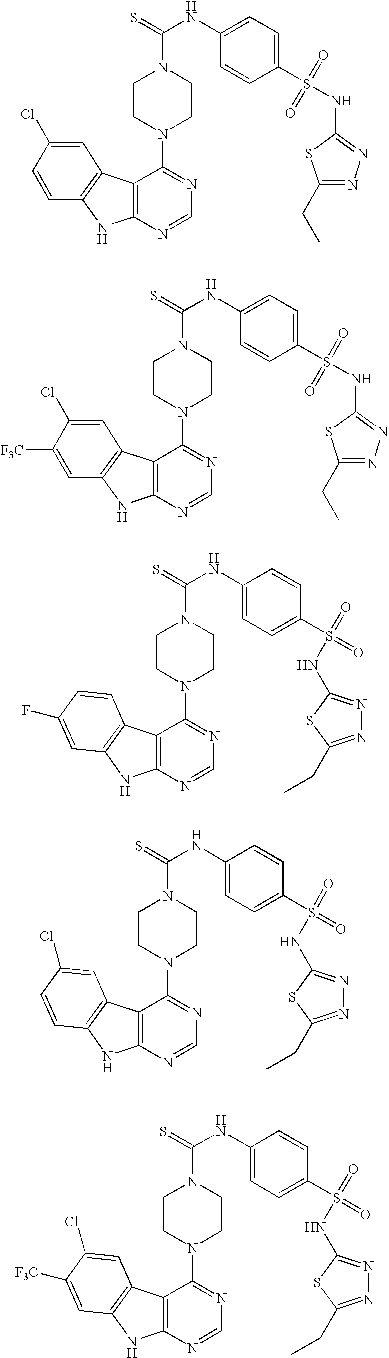

13. The compound of claim 1, where X is NH, Z is CH, L₂ is —C(═S)NH—, R₃ is hydrogen, and the compound has the following structure (VII):

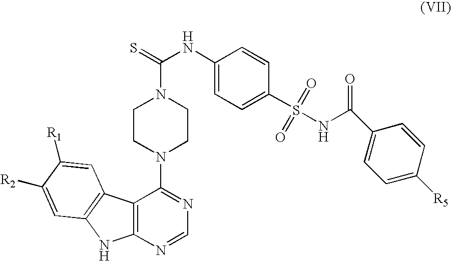

14. The compound of claim 13, where R₁ and R₂ are selected from hydrogen, —OR, —OH, halo, haloalkyl or morpholine, where R is C₁-C₃ alkyl; and where R₅ hydrogen, alkyl, alkoxy, haloalkyl, halo, hydroxyl, or substituted alkoxy.

15. The compound according to claim 14, selected from:

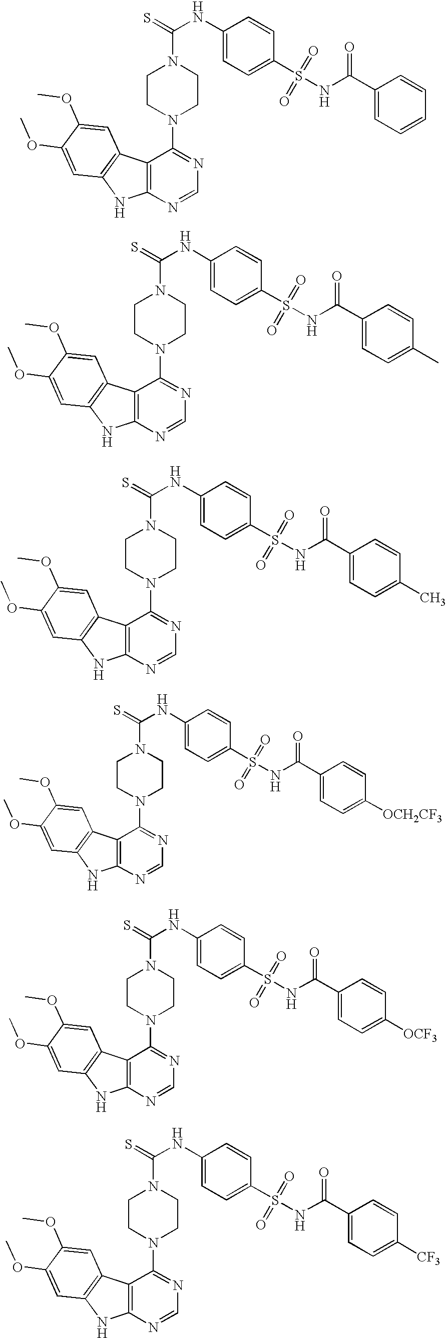

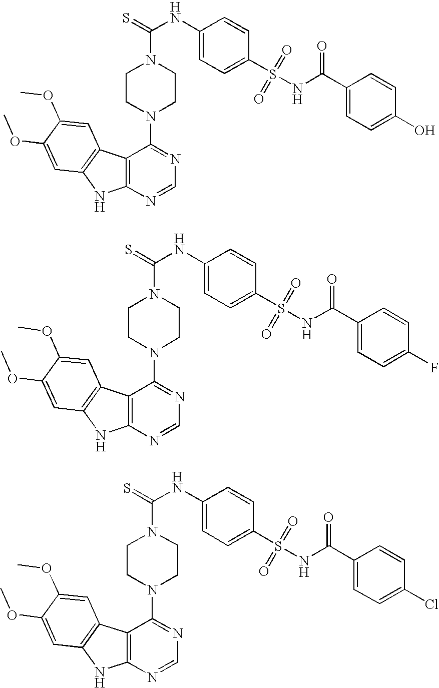

16. The compound of claim 14 having the following structure:

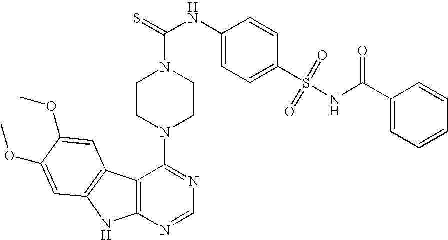

17. A composition comprising a compound of claim 1 in combination with a pharmaceutically acceptable excipient.

18. A method for treating a protein kinase-mediated disease comprising administering to a subject in need thereof a therapeutically effective amount of a composition of claim 17.

19. The method of claim 18, wherein the protein-kinase mediated disease is a cancer.

20. The method of claim 19, wherein the protein-kinase mediated disease is an Aurora kinase-expressing cancer.

21. The method of claim 20, wherein the Aurora kinase-expressing cancer is uterine/cervix squamous cell carcinoma, kidney cancer, small cell lung carcinoma, hepatocellular carcinoma, non-small cell lung carcinoma, melanoma, infiltrating ductal breast cancer, infiltrating lobular breast cancer, lung adenocarcinoma, colorectal cancer, stomach adenocarcinoma, or ovarian adenocarcinoma.

22. A method for inhibiting Aurora kinase-activity comprising administering to a subject in need thereof a therapeutically effective amount of a compound having the following structure (I): 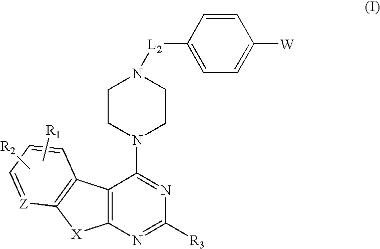
including stereoisomers and pharmaceutically acceptable salts thereof, wherein:
X is NH, S or O;
Z is CH or N;
R₁ and R₂ are the same or different and are independently hydrogen, hydroxyl, halo, —CN, —NO₂, —NH₂, —R, —OR, —SCH₃, —CF₃, morpholine, —C(═O)OR, —OC(═O)R, where R is alkyl or substituted alkyl; or —O(CH₂)ₙ—Rₓ, where n is 2-4 and Rₓ is N-methylpiperazine, morpholine or 2-methylpyrrolidine.
R₃ is hydrogen, —NH₂, alkyl, —CN, or —NO₂, or R₃ is -L₃-Cycl₃ wherein L₃ is a direct bond, —S— or —NH—, and CyCl₃ is a carbocycle, substituted carbocycle, heterocycle or substituted heterocycle; L₃ is —C(═S)NH—,
W is: 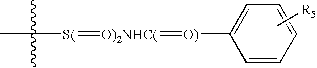
or —S(═O)₂NHR_{y}, where R_{y} is 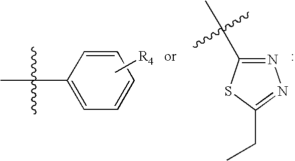
R₄ is alkyl, halo, or haloalkyl; and R₅ is hydrogen, alkyl, alkoxy, haloalkyl, halo, hydroxyl, or substituted alkoxy.

23. The method of claim 22, wherein the administering is oral, subcutaneous or intravenous.

24. The method of claim 22, wherein the administering comprises administering the compound prior to treatment with one or more other chemotherapeutic agents, or in combination with one or more other chemotherapeutic agents.

25. The method of claim 24, wherein the other chemotherapeutic agent is paclitaxel.
